(19) United States
(12) Patent Application Publication
(10) Pub. No.: US 2021/0189384 A1
(43) Pub. Date: Jun. 24, 2021

(54) METHODS AND COMPOSITIONS FOR AMPLICON CONCATENATION

(71) Applicant: Asuragen, Inc., Austin, TX (US)

(72) Inventors: Gary J. LATHAM, Austin, TX (US); Liangjing Chen, Austin, TX (US)

(73) Assignee: Asuragen, Inc., Austin, TX (US)

(21) Appl. No.: 17/104,665

(22) Filed: Nov. 25, 2020

Related U.S. Application Data
(60) Provisional application No. 62/940,537, filed on Nov. 26, 2019.

(51) Int. Cl. C12N 15/10; C12Q 1/6874; C12Q 1/6806

(52) U.S. Cl. CPC C12N 15/1086; C12N 15/1096; C12Q 1/6806; C12Q 1/6874

(57) ABSTRACT

The present disclosure relates to methods and compositions for nucleic acid library preparation. In certain aspects, the present disclosure relates to methods of making a library of concatenated amplicons from a target nucleic acid. The present disclosure further relates to methods of using the methods and compositions described herein, e.g., in downstream applications such as sequencing (e.g., single-molecule sequencing), gene assembly, and/or structural variation characterization.

The present disclosure relates to methods and compositions for nucleic acid library preparation and their use in sequencing applications. In certain aspects, the present disclosure relates to methods of making a library of concatenated amplicons from a target nucleic acid. In some embodiments, the libraries disclosed and generated by the methods described herein may be useful in various downstream applications, such as analyzing and characterizing the molecular features of genomic targets. Compositions and kits for making a library of concatenated amplicons (e.g., using any of the exemplary methods described herein) are also provided.

Since the advent of “second-generation” sequencing (or next-generation sequencing), the cost of genome sequencing has precipitately dropped (Mardis, (2008) Trends Genet. 24(3):133-41). These technologies, which can produce short reads a few hundred base pairs in length, have enabled the sequencing of many new genomes along with widespread resequencing efforts to analyze genomic diversity (Schatz et al., (2010) Genome Res. 20(9):1165-73; 1000 Genomes Project Consortium, (2010) Nature 467(7319):1061-73). Although second-generation sequencing has enabled population-scale analyses of single nucleotide and other small variants, analysis of larger structural variations has proved difficult. Further, new genomes assembled de novo using second-generation technologies are often of lower quality compared with those genomes sequenced using older, more expensive methods (International Rice Genome Sequencing Project, (2005) Nature 436(7052):793-800; Lander et al., (2001) Nature 409(6822):860-921). Resequencing projects may also be limited in their analysis of structural variations, missing tens of thousands of structural variants or more per mammalian-sized genome (Chaisson et al., (2015) Nature 517(7536):608-11).

The availability of “third-generation” single-molecule sequencing technologies that are affordable for many laboratories and can produce average read lengths of more than 10,000 base pairs has enabled improved analysis of genome structure (Lee et al., (2016) “Third-generation sequencing and the future of genomics,” DOI: 10.1101/048603). With respect to structural variation analysis, long reads improve “split-read” analyses such that insertions, deletions, translocations, and other structural changes can be more readily recognized (Chaisson et al., (2015) Nature 517(7536):608-11). Single-molecule sequencing technologies can also produce more uniform coverage of the genome since as they are not as sensitive to GC- or AT-biased content as second-generation technologies, which tend to have reduced or completely absent coverage over regions with imbalanced sequence composition (Ross et al., (2013) Genome Biol. 14(5):R51). Additional advantages of single-molecule sequencing include single-molecule sensitivity and continuous or real-time readouts.

Long-read technologies, such as single-molecule real-time (SMRT®) technology (Pacific Biosciences, Menlo Park, Calif.) and nanopore-based methods (Oxford Nanopore Technologies, Oxford, UK), address several limitations of short-read sequencers. However, long-read technologies still suffer from low throughput (ranging from about 100,000 to about 10 million reads) compared to competing short-read sequencing platforms, in addition to a variable raw error rate (up to about 10-20%). Long-read technologies have also been hampered by sample and preparation methods that are not suitable for long-read sequencing, such as those for oncology and prenatal testing applications, which typically use short nucleic acid fragments such as cell-free DNA (cfDNA) or circulating tumor DNA (ctDNA) present in trace amounts in blood (Newman et al., (2014) Nat Med. 20(5):548-54). Thus, novel sample preparation strategies capable of providing long DNA templates could increase the throughput of single-molecule sequencing platforms. Such methods could also increase the versatility of these platforms to cost-effectively sequence both long and short DNA molecules.

Molecular biology methods designed to generate long DNA templates by concatenating DNA fragments into genes or gene clusters have been proposed. See, e.g., WO 2018/108328; Schlecht et al., (2017) Scientific Reports 7:5252; Kadkhodaei et al., (2016) RSC Adv. 6:66682-94; Mitani et al., (2004) BioTechniques 37(1):124-9; Ramteke et al., (2016) F1000Research 4:160; Marcozzi et al., (2019) “CyclomicsSeq a sensitive liquid biopsy genetic test real-time and cost-efficient cancer monitoring in blood”). However, current methods, such as those using Gibson Assembly to covalently link DNA fragments with complementary ends, have limitations, including (i) a requirement for a minimum fragment size; (ii) assembly of amplicons in a random order; (iii) a wide distribution of product size; (iv) the ability to only assemble up to about 5 amplicons; and/or (v) a requirement for a purification step between any amplicon synthesis and assembly reactions. Thus, there remains a need for more effective methods of library preparation, particularly those that are capable of harnessing the advantages of long-read single-molecule sequencing platforms and may also be applied to other downstream applications (e.g., gene assembly, molecular characterization of sequence variations, etc.).

The present disclosure provides, in part, novel methods and compositions for nucleic acid library preparation and improved sequencing/sequence assembly methods. In certain aspects, the present disclosure provides methods and compositions for concatenating multiple discrete amplicons into one or more longer amplicons. In certain aspects, the present disclosure provides a method of making a library of concatenated amplicons from a target nucleic acid by generating tagged amplicons from the target nucleic acid (e.g., by amplifying two or more regions of interest (ROIs)); concatenating the tagged amplicons to generate one or more concatenated amplicons; and amplifying the one or more concatenated amplicons to generate a library of concatenated amplicons. In some embodiments, each ROI is amplified with a forward primer and a reverse primer. In some embodiments, each primer comprises a 5′ tag sequence and a sequence capable of hybridizing to an ROI. In some embodiments, the 5′ tag sequence of the reverse primer for each ROI is complementary to the 5′ tag sequence of the forward primer for another ROI.

In some embodiments, amplicons are designed to enrich genomic sequences of interest (e.g., exons). In some embodiments, enrichment of such genomic sequences allows sequencing reads and/other downstream analyzers to focus on regions of interest and exclude other regions (e.g., non-coding sequences, e.g., introns). Thus, in some embodiments, enrichment may result in time and/or cost savings. In some embodiments, amplicons are concatenated in a predetermined order. In some embodiments, amplicons are concatenated such that the assembled concatemer comprises single-copy representation of each amplicon.

In some embodiments, the methods and compositions disclosed herein may be useful in various downstream applications. An exemplary application of the disclosed methods and compositions is sequencing analysis, e.g., using single-molecule sequencing. In some embodiments, the methods and compositions disclosed herein provide one or more advantages over alternate methods for nucleic acid library preparation and/or related sequencing using such a library (e.g., those using Gibson assembly for amplicon concatenation). Exemplary advantages include, without limitation: (i) no restriction on fragment size, thereby providing compatibility with short, degraded samples, such as formalin-fixed paraffin-embedded (FFPE) or cell-free DNA (liquid biopsy) samples; (ii) a self-normalizing workflow capable of generating a product with a defined size and amplicons concatenated in a uniform (e.g., 1:1) stoichiometry; (iii) ability to concatenate more amplicons (e.g., more than 5 amplicons); (iv) no requirement for a purification step between any amplicon synthesis and assembly reactions; (v) reduction in time and/or cost for sample preparation; and (vi) increased throughput for downstream applications (e.g., single-molecule sequencing, e.g., cost-effective multiple gene sequencing assays that can be configured on a single flow cell). In some embodiments, the methods and compositions disclosed herein provide effective strategies for nucleic acid library preparation that can be applied to sequencing across panels of different genes and/or markers.

In some embodiments, the methods and compositions disclosed herein increase the size of multiple discrete amplicons via amplicon concatenation. In some embodiments, the amplicon concatenation methods described herein generate concatemer templates suitably sized for downstream applications (e.g., using single-molecule sequencing). In some embodiments, the amplicon concatenation methods described herein may increase throughput of single-molecule sequencing by up to about 50-fold, up to about 100-fold, or more, as compared to alternate methods for nucleic acid library preparation. In some embodiments, the methods and compositions described herein may have advantages not only for sequencing analysis, but also for other downstream applications. Exemplary potential applications include gene assembly and molecular characterization of sequence variations (e.g., single nucleotide variants (SNV), indels, gene chimera, and copy number changes) within target loci, e.g., using analyzers other than single-molecule sequencing platforms.

In some embodiments, the present disclosure provides a method of making a library of concatenated amplicons from a target nucleic acid, the method comprising:
i. generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons; and
iii. amplifying the one or more concatenated amplicons to generate a library of concatenated amplicons.

In some embodiments, amplifying two or more ROIs comprises polymerase chain reaction (PCR) or isothermal amplification. In some embodiments, amplifying two or more ROIs comprises PCR. In some embodiments, amplifying two or more ROIs comprises multiplex PCR. In some embodiments, PCR and/or multiplex PCR comprises magnesium in a working concentration of about 0.5 mM to about 4 mM. In some embodiments, PCR and/or multiplex PCR comprises magnesium in a working concentration of about 1 mM to about 3.5 mM. In some embodiments, PCR and/or multiplex PCR comprises magnesium in a working concentration of about 1.5 mM to about 3 mM. In some embodiments, PCR and/or multiplex PCR comprises dimethyl sulfoxide (DMSO) in a working concentration of about 1% to about 8% by volume (v/v). In some embodiments, PCR and/or multiplex PCR comprises DMSO in a working concentration of about 3% to about 6% by volume. In some embodiments, PCR and/or multiplex PCR comprises a pH of about 8 to about 10. In some embodiments, PCR and/or multiplex PCR comprises a pH of about 8.5 to about 9.2.

In some embodiments, amplifying two or more ROIs comprises amplifying at least two, at least 5, at least 10, at least 20, at least 30, at least 40, or at least 50 ROIs. In some embodiments, amplifying two or more ROIs comprises amplifying at least 2, 3, 4, 5, 6, 7, 8, 9, 10, or more, e.g., at least 12, or at least 14 ROIs. In some embodiments, each ROI is about 2, about 5, about 10, about 20, about 30, about 40, about 50, about 100, about 150, about 200, about 250, about 500, about 1,000, about 2,000, about 5,000, or about 10,000 nucleotides in length.

In some embodiments, the working concentration of one or more primers in step (i) is about 1 nM to about 5,000 nM (e.g., about 10 nM to about 100 nM, e.g., about 30 nM). In some embodiments, the working concentration of one or more primers in step (i) is about 10 nM to about 100 nM (e.g., about 30 nM). In some embodiments, the working concentration of one or more primers in step (i) is about 30 nM.

In some embodiments, one or more primers in step (i) are depleted prior to concatenating the tagged amplicons. In some embodiments, one or more primers in step (i) are selected to prevent formation of one or more primer dimers. In some embodiments, the one or more primers lack 5 or more (e.g., 5, 6, 7, 8, or more) exactly-matched bases at the 3′ end of the primer sequences. In some embodiments, the one or more primers prevent formation of one or more primer dimers (e.g., one or more exponential amplifiable primer dimers). In some embodiments, the one or more primers lack 7 or more (e.g., 7. 8, 9, 10, or more) exactly-matched bases at the 3′ end of the primer sequences. In some embodiments, the one or more primers prevent formation of one or more primer dimers (e.g., one or more linear amplifiable primer dimers). In some embodiments, one or more primers in step (i) comprise minimal sequence that is capable of hybridizing to an ROI and also complementary to a sequence in another primer. In some embodiments, the minimal sequence is about 6 to about 100 nucleotides in length, e.g., about 6 to about 50 or about 15 to about 30 nucleotides in length, e.g., about 18 to about 20 nucleotides in length. In some embodiments, the minimal sequence is about 6 to about 50 nucleotides in length, e.g., about 6 to about 30 or about 15 to about 30 nucleotides in length, e.g., about 18 to about 20 nucleotides in length. In some embodiments, the minimal sequence is about 6 to about 30 nucleotides in length. In some embodiments, the minimal sequence is about 4 to about 40, about 5 to about 35, or about 6 to about 30 nucleotides in length. In some embodiments, the minimal sequence is about 10, about 15, about 20, about 25, about 30, or about 35 nucleotides in length. In some embodiments, the minimal sequence is about 15 to about 30 nucleotides in length. In some embodiments, the minimal sequence is about 18 to about 20 nucleotides in length. In some embodiments, the minimal sequence is at least about 4, about 5, about 6, about 7, about 8, about 9, or about 10 nucleotides in length. In some embodiments, the minimal sequence is at least about 6 nucleotides in length.

In some embodiments, one or more primers in step (i) are selected to minimize formation of one or more dead-end intermediate products. In some embodiments, the one or more dead-end intermediate products cannot form one or more concatenated amplicons. In some embodiments, one or more primers in step (i) comprise at least one adenine between the 5′ tag sequence and the sequence capable of hybridizing to the ROI. In some embodiments, one or more primers in step (i) comprise a 5′ phosphate. In some embodiments, one or more primers in step (i) comprise a molecular barcode. In some embodiments, the 5′ tag sequence in one or more primers is an artificial tag sequence. In some embodiments, the artificial tag sequence is not homologous to a human genome sequence.

In some embodiments, the tagged amplicons are not purified prior to concatenation. In some embodiments, concatenating the tagged amplicons comprises providing a DNA polymerase. In some embodiments, the DNA polymerase has 3′ to 5′ exonuclease activity. In some embodiments, the DNA polymerase is a high-fidelity DNA polymerase. In some embodiments, the DNA polymerase is a Q5, Pfu, or Kapa HiFi HotStart DNA polymerase. In some embodiments, concatenating the tagged amplicons comprises providing at least one adjuvant. In some embodiments, the at least one adjuvant comprises TMAC, ThermaGo, and/or ThermaStop.

In some embodiments, concatenating the tagged amplicons comprises concatenating at least two, at least 5, at least 10, at least 20, at least 30, at least 40, or at least 50 tagged amplicons. In some embodiments, each tagged amplicon is about 50, about 100, about 150, about 200, about 250, about 500, about 1,000, about 2,000, about 5,000, or about 10,000 nucleotides in length. In some embodiments, the total length of the one or more concatenated amplicons is about 2,000 to about 50,000 nucleotides. In some embodiments, the total length of the one or more concatenated amplicons is about 2,000 to about 20,000 nucleotides. In some embodiments, the total length of the one or more concatenated amplicons is about 10,000 nucleotides. In some embodiments, the total length of the one or more concatenated amplicons is about 5,000 nucleotides. In some embodiments, the total length of the one or more concatenated amplicons is about 3,000 to about 4,000 nucleotides.

In some embodiments, the one or more concatenated amplicons are in a predetermined order. In some embodiments, the predetermined order results from the tag sequences in the primers. In some embodiments, the 5′ tag sequence of the reverse primer for each ROI is complementary to the 5′ tag sequence of the forward primer for the ROI immediately downstream. In some embodiments, the order of the one or more concatenated amplicons is identical to the order of the corresponding ROIs in the target nucleic acid.

In some embodiments, the one or more concatenated amplicons comprise single-copy representation of each tagged amplicon. In some embodiments, the ratio of the one or more concatenated amplicons to the corresponding ROIs in the target nucleic acid is about 1 to 1.

In some embodiments, amplifying the one or more concatenated amplicons comprises PCR and/or multiplex PCR. In some embodiments, the PCR and/or multiplex PCR conditions comprise magnesium. In some embodiments, the magnesium is in a working concentration of about 0.5 mM to about 4 mM. In some embodiments, PCR and/or multiplex PCR comprises magnesium, e.g., in a working concentration of about 1 mM to about 3.5 mM. In some embodiments, PCR and/or multiplex PCR comprises magnesium in a working concentration of about 1.5 mM to about 3 mM. In some embodiments, the PCR and/or multiplex PCR conditions comprise DMSO. In some embodiments, the DMSO is in a working concentration of about 1% to about 8% by volume. In some embodiments, PCR and/or multiplex PCR comprises DMSO in a working concentration of about 3% to about 6% by volume. In some embodiments, the PCR and/or multiplex PCR conditions comprise a pH of about 8 to about 10. In some embodiments, PCR and/or multiplex PCR comprises a pH of about 8.5 to about 9.2.

In some embodiments, amplifying the one or more concatenated amplicons comprises a first end primer capable of hybridizing to a tag sequence at the 5′ end of a concatenated amplicon and a second end primer capable of hybridizing to a tag sequence at the 3′ end of a concatenated amplicon. In some embodiments, the tag sequence at the 5′ end of the concatenated amplicon is identical to or overlaps with the 5′ tag sequence of a forward primer used to amplify an ROI in step (i). In some embodiments, the tag sequence at the 3′ end of the concatenated amplicon is identical to or overlaps with the 5′ tag sequence of a reverse primer used to amplify an ROI in step (i). In some embodiments, the first end primer and the second end primer are added in any one of steps (i)-(iii). In some embodiments, the first end primer and the second end primer are added in step (i). In some embodiments, the first end primer and the second end primer are added in step (ii) or step (iii).

In some embodiments, a method described herein (e.g., a method of making a library of concatenated amplicons) further comprises analyzing a library of concatenated amplicons. In some embodiments, analyzing comprises sequencing, gene assembly, and/or structural variation characterization.

In some embodiments, sequencing comprises single-molecule sequencing. In some embodiments, sequencing comprises long-read sequencing. In some embodiments, sequencing comprises sequencing about 800 nucleotides or longer. In some embodiments, sequencing comprises nanopore sequencing or single-molecule real-time (SMRT) sequencing. In some embodiments, structural variation characterization comprises detecting or quantifying single nucleotide variants (SNV), repeat sequences, indels, gene chimera, and/or gene copy number. In some embodiments, detecting or quantifying gene copy number comprises detecting or quantifying one or more molecular barcodes. In some embodiments, the one or more molecular barcodes are in one or more primers in step (i). In some embodiments, detecting or quantifying gene copy number comprises using and/or comparing to an external spiking control. In some embodiments, the external spiking control comprises a synthetic gBlock control. In some embodiments, structural variation characterization comprises labeling and/or direct imaging.

In some embodiments, a target nucleic acid comprises one or more genes or a multiple gene panel. In some embodiments, the one or more genes comprise a human gene. In some embodiments, the human gene is a human disease gene. In some embodiments, the human gene is a human cancer gene. In some embodiments, the one or more genes comprise CFTR, SMN1, SMN2, KRAS, BRAF, PIK3C, EGFR, and/or ERBB2. In some embodiments, the human gene is a human gene with high modeled fetal disease risk (MFDR). In some embodiments, the one or more genes comprise SMN1, SMN2, FMR1, HBA1, HBA2, and/or GBA. In some embodiments, the one or more genes comprise CFTR, FMR1, SMN1, SMN2, IKBKAP, ABCC8, FANCC, GALT, GBA, G6PC, HBA1, HBA2, HBB, BLM, ASPA, TMEM216, BCKDHA, BCKDHB, ACADM, MCOLN1, NEB, SMPD1, F8, HEXA, PCDH15, DMD, CYP21A2, and/or CLRN1. In some embodiments, the one or more genes comprise CFTR, FMR1, SMN1, and/or SMN2.

In some embodiments, a target nucleic acid is used in a multiple gene panel. In some embodiments, the multiple gene panel is a newborn or carrier screening panel. In some embodiments, the multiple gene panel comprises a human gene. In some embodiments, the multiple gene panel comprises at least about 20 human genes (e.g., at least about 22 human genes). In some embodiments, the multiple gene panel comprises at least about 22 human genes. In some embodiments, the human gene is a human disease gene. In some embodiments, the human gene is a human cancer gene. In some embodiments, the multiple gene panel comprises CFTR, SMN1, SMN2, KRAS, BRAF, PIK3C, EGFR, and/or ERBB2. In some embodiments, the human gene is a human gene with high modeled fetal disease risk (MFDR) In some embodiments, the multiple gene panel comprises SMN1, SMN2, FMR1, HBA1, HBA2, and/or GBA. In some embodiments, the multiple gene panel comprises CFTR, FMR1, SMN1, SMN2, IKBKAP, ABCC8, FANCC, GALT, GBA, G6PC, HBA1, HBA2, HBB, BLM, ASPA, TMEM216, BCKDHA, BCKDHB, ACADM, MCOLN1, NEB, SMPD1, F8, HEXA, PCDH15, DMD, CYP21A2, and/or CLRN1. In some embodiments, the multiple gene panel comprises CFTR, FMR1, SMN1, and/or SMN2.

In some embodiments, a target nucleic acid is from a biological sample (e.g., a liquid and/or biopsy sample). In some embodiments, the biological sample comprises a blood sample. In some embodiments, the biological sample comprises a buccal sample. In some embodiments, the biological sample comprises a biopsy sample. In some embodiments, the biopsy sample comprises frozen tissue or formalin-fixed paraffin-embedded (FFPE) tissue. In some embodiments, the biopsy sample comprises a liquid biopsy sample. In some embodiments, the liquid biopsy sample comprises cell-free DNA or DNA from circulating tumor cells (i.e., circulating tumor DNA (ctDNA)).

The present disclosure further provides, in some embodiments, a library of concatenated amplicons, wherein the library is made by:
i. generating tagged amplicons by amplifying two or more regions of interest (ROIs) from a target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons; and
iii. amplifying the one or more concatenated amplicons to generate a library of concatenated amplicons.

Further provided herein, in some embodiments, is a method of selecting a set of primers capable of amplifying two or more regions of interest (ROIs) from a target nucleic acid, comprising selecting a forward primer and a reverse primer for each ROI, wherein each primer comprises a 5′ tag sequence and a sequence capable of hybridizing to the ROI, and wherein:
a) the 5′ tag sequence of the reverse primer for each ROI is complementary to the 5′ tag sequence of the forward primer for another ROI;
b) the 5′ tag sequence is an artificial tag sequence; and
c) each primer comprises minimal sequence that is capable of hybridizing to an ROI and also complementary to a sequence in another primer.

Further provided herein, in some embodiments, is a kit comprising a set of primers and instructions for use of the primers in amplifying two or more regions of interest (ROIs) from a target nucleic acid, wherein the set of primers comprises a forward primer and a reverse primer for each ROI, wherein each primer comprises a 5′ tag sequence and a sequence capable of hybridizing to the ROI, and wherein:
a) the 5′ tag sequence of the reverse primer for each ROI is complementary to the 5′ tag sequence of the forward primer for the ROI immediately downstream:
b) the 5′ tag sequence is an artificial tag sequence; and each primer comprises minimal sequence that is capable of hybridizing to an ROI and also complementary to a sequence in another primer.

In some embodiments of the methods and compositions (e.g., libraries, kits) described herein, one or more primers (e.g., all primers) comprise minimal sequence that is capable of hybridizing to an ROI. In some embodiments, one or more primers (e.g., all primers) comprise minimal sequence that is complementary to a sequence in another primer. In some embodiments, one or more primers (e.g., all primers) comprise minimal sequence that is capable of hybridizing to an ROI and also complementary to a sequence in another primer. In some embodiments, the minimal sequence is about 6 to about 100 nucleotides in length, e.g., about 6 to about 50 or about 15 to about 30 nucleotides in length, e.g., about 18 to about 20 nucleotides in length. In some embodiments, the minimal sequence is about 6 to about 50 nucleotides in length, e.g., about 6 to about 30 or about 15 to about 30 nucleotides in length, e.g., about 18 to about 20 nucleotides in length. In some embodiments, the minimal sequence is about 6 to about 30 nucleotides in length. In some embodiments, the minimal sequence is about 4 to about 40, about 5 to about 35, or about 6 to about 30 nucleotides in length. In some embodiments, the minimal sequence is about 10, about 15, about 20, about 25, about 30, or about 35 nucleotides in length. In some embodiments, the minimal sequence is about 15 to about 30 nucleotides in length. In some embodiments, the minimal sequence is about 18 to about 20 nucleotides in length. In some embodiments, the minimal sequence is at least about 4, about 5, about 6, about 7, about 8, about 9, or about 10 nucleotides in length. In some embodiments, the minimal sequence is at least about 6 nucleotides in length. In some embodiments, one or more primers comprise at least one adenine between the 5′ tag sequence and the sequence capable of hybridizing to the ROI. In some embodiments, one or more primers comprise a 5′ phosphate. In some embodiments, one or more primers comprise a molecular barcode. In some embodiments, the artificial tag sequence is not homologous to a human genome sequence.

Also provided herein, in some embodiments, is a method of sequencing a library of concatenated amplicons, wherein the library of concatenated amplicons is made by any of the exemplary methods described herein.

Also provided herein, in some embodiments, is a method of sequencing a target nucleic acid, the method comprising:
i. generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons;
iii. amplifying the one or more concatenated amplicons to generate a library of concatenated amplicons; and
iv. sequencing the library of concatenated amplicons.

In some embodiments of the methods (e.g., the sequencing methods) described herein, amplifying two or more ROIs comprises amplifying at least two, at least 5, at least 10, at least 20, at least 30, at least 40, or at least 50 ROIs. In some embodiments, amplifying two or more ROIs comprises amplifying at least 2, 3, 4, 5, 6, 7, 8, 9, 10, or more, e.g., at least 12, or at least 14 ROIs. In some embodiments, each ROI is about 2, about 5, about 10, about 20, about 30, about 40, about 50, about 100, about 150, about 200, about 250, about 500, about 1,000, about 2,000, about 5,000, or about 10,000 nucleotides in length.

In some embodiments, concatenating the tagged amplicons comprises concatenating at least two, at least 5, at least 10, at least 20, at least 30, at least 40, or at least 50 tagged amplicons. In some embodiments, each tagged amplicon is about 50, about 100, about 150, about 200, about 250, about 500, about 1,000, about 2,000, about 5,000, or about 10,000 nucleotides in length. In some embodiments, the total length of the one or more concatenated amplicons is about 2,000 to about 50,000 nucleotides. In some embodiments, the total length of the one or more concatenated amplicons is about 2,000 to about 20,000 nucleotides. In some embodiments, the total length of the one or more concatenated amplicons is about 10,000 nucleotides. In some embodiments, the total length of the one or more concatenated amplicons is about 5,000 nucleotides. In some embodiments, the total length of the one or more concatenated amplicons is about 3,000 to about 4,000 nucleotides.

In some embodiments, the one or more concatenated amplicons are in a predetermined order. In some embodiments, the predetermined order results from the tag sequences in the primers. In some embodiments, the 5′ tag sequence of the reverse primer for each ROI is complementary to the 5′ tag sequence of the forward primer for the ROI immediately downstream. In some embodiments, the order of the one or more concatenated amplicons is identical to the order of the corresponding ROIs in the target nucleic acid.

In some embodiments, the one or more concatenated amplicons comprise single-copy representation of each tagged amplicon. In some embodiments, the ratio of the one or more concatenated amplicons to the corresponding ROIs in the target nucleic acid is about 1 to 1.

In some embodiments, sequencing comprises single-molecule sequencing. In some embodiments, sequencing comprises long-read sequencing. In some embodiments, sequencing comprises sequencing about 800 nucleotides or longer. In some embodiments, sequencing comprises nanopore sequencing or single-molecule real-time (SMRT) sequencing.

In some embodiments, a method described herein (e.g., a method of sequencing a target nucleic acid) further comprises analyzing a library of concatenated amplicons before, during, or after sequencing. In some embodiments, analyzing comprises gene assembly and/or structural variation characterization. In some embodiments, structural variation characterization comprises detecting or quantifying single nucleotide variants (SNV), repeat sequences, indels, gene chimera, and/or gene copy number. In some embodiments, detecting or quantifying gene copy number comprises detecting or quantifying one or more molecular barcodes. In some embodiments, the one or more molecular barcodes are in one or more primers in step (i). In some embodiments, detecting or quantifying gene copy number comprises using and/or comparing to an external spiking control. In some embodiments, the external spiking control comprises a synthetic gBlock control. In some embodiments, structural variation characterization comprises labeling and/or direct imaging.

In some embodiments, a target nucleic acid comprises one or more genes or a multiple gene panel. In some embodiments, the one or more genes comprise a human gene. In some embodiments, the human gene is a human disease gene. In some embodiments, the human gene is a human cancer gene. In some embodiments, the one or more genes comprise CFTR, SMN1, SMN2, KRAS, BRAF, PIK3C, EGFR, and/or ERBB2. In some embodiments, the human gene is a human gene with high modeled fetal disease risk (MFDR). In some embodiments, the one or more genes comprise SMN1, SMN2, FMR1, HBA1, HBA2, and/or GBA. In some embodiments, the one or more genes comprise CFTR, FMR1, SMN1, SMN2, IKBKAP, ABCC8, FANCC, GALT, GBA, G6PC, HBA1, HBA2, HBB, BLM, ASPA, TMEM216, BCKDHA, BCKDHB, ACADM, MCOLN1, NEB, SMPD1, F8, HEXA, PCDH15, DMD, CYP21A2, and/or CLRN1. In some embodiments, the one or more genes comprise CFTR, FMR1, SMN1, and/or SMN2.

In some embodiments, a target nucleic acid is used in a multiple gene panel. In some embodiments, the multiple gene panel is a newborn or carrier screening panel. In some embodiments, the multiple gene panel comprises a human gene. In some embodiments, the multiple gene panel comprises at least about 20 human genes (e.g., at least about 22 human genes). In some embodiments, the multiple gene panel comprises at least about 22 human genes. In some embodiments, the human gene is a human disease gene. In some embodiments, the human gene is a human cancer gene. In some embodiments, the multiple gene panel comprises CFTR, SMN1, SMN2, KRAS, BRAE, PIK3C, EGFR, and/or ERBB2. In some embodiments, the human gene is a human gene with high modeled fetal disease risk (MFDR). In some embodiments, the multiple gene panel comprises SMN1, SMN2, FMR1, HBA1, HBA2, and/or GBA. In some embodiments, the multiple gene panel comprises CFTR, FMR1, SMN1, SMN2, IKBKAP, ABCC8, FANCC, GALT, GBA, G6PC, HBA1, HBA2, HBB, BLM, ASPA, TMEM216, BCKDHA, BCKDHB, ACADM, MCOLN1, NEB, SMPD1, F8, HEXA, PCDH15, DMD, CYP21A2, and/or CLRN1. In some embodiments, the multiple gene panel comprises CFTR, FMR1, SMN1, and/or SMN2.

### BRIEF DESCRIPTION OF THE DRAWINGS

FIG. 1 shows an exemplary amplicon concatenation method of amplifying a sequence of interest.

FIG. 2A shows the observed capillary electrophoresis (CE) size and CE trace of a 1^{st} 6-amplicon concatenation. FIG. 2B shows the observed CE size and CE trace of a 2^{nd} 6-amplicon concatenation.

FIG. 3 shows the CE trace of an assembled 12-amplicon concatenation product assembled from two gel-purified fragments of the 1^{st} and the 2^{nd} 6-amplicon concatenation in FIG. 2A and FIG. 2B, respectively.

FIG. 4A shows an exemplary primer redesign to eliminate an exponentially-amplifiable primer dimer, Upper: Formation of a 78 bp primer dimer can result in a 80 bp deletion in the 2^{nd} 6-amplicon concatenation. Lower: Redesigned primers cannot form a primer dimer due to the presence of only 2 perfectly matched bases at the 3′ end of the primers. FIG. 4B shows an exemplary primer redesign to eliminate an off-target amplification. T13354/T13359 primers can form a 121 bp non-specific PCR product and result in a 260 bp deletion product in the 2^{nd} 6-amplicon concatenation. Substitution of T13354 with T14642 can eliminate this deletion product. FIG. 4C shows an exemplary primer redesign to eliminate a linearly-amplifiable primer dimer. The T13357 primer can hybridize and extend on primer T13344 (10 perfectly matched bases) to form a 51 bp primer dimer with linear amplification. This can cause a 748 bp deletion in the final 12-amplicon concatenation product. Substitution of T13357 with T14391 can eliminate the primer dimer and result in observation of the final, single band full length 12-amplicon concatenation product. FIG. 4D shows the CE trace of a 2^{nd} 6-amplicon concatenation. FIG. 4E shows the CE trace of an assembled 12-amplicon concatenation product. FIG. 4F shows the CE trace of an assembled 12-amplicon concatenation product with primers designed to avoid primer dimers and non-specific amplification.

FIG. 5 shows the CE trace of an assembled 4-amplicon concatenation product from the CFTR gene, including detection of a 297 nucleotide 1^{st} fragment peak.

FIG. 6A-6D show the CE trace of an exemplary assembled 4-amplicon concatenation product following multiplex PCR using a final primer concentration of 40 nM (FIG. 6A), 30 nM (FIG. 6B), 10 nM (FIG. 6C), or 5 nM (FIG. 6D).

FIG. 7 shows an exemplary scenario for inserting an extra thymine (T) in a DNA template, e.g., to accommodate a potential 3′ adenine (A) overhang.

FIG. 8 shows the CE trace of an assembled 4-amplicon concatenation product from the CFTR gene.

FIG. 9A-9D show the CE trace of exemplary assembled 4- or 6-amplicon concatenation products following multiplex PCR with Kapa HiFi HotStart DNA polymerase. PCR conditions: with extra A in primer, without additive (FIG. 9A); with extra A in primer, with TMAC and ThermaStop additives (FIG. 9B); without extra A in primer, with TMAC, ThermaGo, and ThermaStop additives (FIG. 9C); and without extra A in primer, with TMAC and ThermaStop additives (FIG. 9D).

FIG. 10 shows the CE trace of an assembled 6-amplicon concatenation product from the CFTR gene.

FIG. 11A shows an agarose gel analysis of a 6-amplicon concatenation using 10, 15, 20, or 25 cycles of multiplex PCR. FIG. 11B shows the CE trace and agarose gel of an assembled 14-amplicon concatenation product from the CFTR gene. FIG. 11C shows an Integrative Genomics Viewer (IGV) view of the full length 3203 nt concatenation constructs confirmed by nanopore sequencing.

FIG. 12A shows an exemplary experimental design for co-detection of CFTR variants, and SMN1/SMN2 copy number variation, disease modifiers, and/or silent carrier mutations. FIG. 12B shows a sequence alignment of artificial CFTR* and SMN* gBlock sequence with natural genomic sequence. Differential bases are shown in rectangular boxes. FIG. 12C shows the CE trace and agarose gel of the assembled CFTR 6-amplicon+SMN amplicon concatenation product. FIG. 12D shows the linear correlation of the SMN1/SMN2 ratio from concatenation/nanopore sequencing and the AmplideX® PCR/CE SMN1/2 Kit (RUO).

### DETAILED DESCRIPTION OF ILLUSTRATIVE EMBODIMENTS

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein are also incorporated by reference in their entirety. To the extent a cited reference conflicts with the disclosure herein, the specification shall control.

As used herein, the singular forms of a word also include the plural form, unless the context clearly dictates otherwise. As examples, the terms “a,” “an,” and “the” are understood to be singular or plural. Likewise, “an element” means one or more element. The term “or” shall mean “and/or” unless the specific context indicates otherwise. All ranges include the endpoints and all points in between unless the context indicates otherwise.

The term “about” or “approximately,” as used herein in the context of numerical values and ranges, refers to values or ranges that approximate or are close to the recited values or ranges such that the embodiment may perform as intended, as is apparent to the skilled person from the teachings contained herein. Thus, these terms encompass values beyond those resulting from systematic error. In some embodiments, “about” or “approximately” means plus or minus 10% of a numerical amount.

### Methods and Compositions

In certain aspects, the present disclosure provides methods and compositions for nucleic acid library preparation. In certain aspects, the methods and compositions disclosed herein are used in various downstream applications (e.g., single-molecule sequencing, gene assembly, structural variation characterization, etc,).

In some embodiments, the methods and compositions disclosed herein relate to the concatenation of multiple discrete amplicons into one or more longer amplicons. In some embodiments, the methods disclosed herein comprise generating tagged amplicons, concatenating tagged amplicons, and/or amplifying one or more concatenated amplicons. In some embodiments, generating tagged amplicons comprises amplifying two or more regions of interest (ROIs) from a target nucleic acid, e.g., using tagged, gene-specific primers. In some embodiments, generating tagged amplicons comprises PCR (e.g., multiplex PCR, e.g., multiplex overlap extension (MOE)-PCR).

In some embodiments, the tagged amplicons are assembled by concatenation into one or more longer amplicons. In some embodiments, the one or more concatenated amplicons comprise multiple shorter amplicons in a predetermined order. In some embodiments, the predetermined order results from the tag sequences in the gene-specific primers used for amplification. In some embodiments, the one or more concatenated amplicons comprise single-copy representation (e.g., a defined unitary copy number) of each tagged amplicon. In some embodiments, the methods and related compositions (e.g., libraries, kits) disclosed herein offer one or more benefits for nucleic acid library preparation, including but not limited to increased simplicity, scale, and/or specificity. In some embodiments, the methods and related compositions (e.g., libraries, kits) disclosed herein may be useful in various downstream applications, such as sequencing (e.g., single-molecule sequencing, e.g., nanopore sequencing or single-molecule real-time (SMRT) sequencing). Other exemplary applications for the disclosed methods and compositions include, without limitation, gene assembly and molecular characterization of sequence variations (e.g., single nucleotide variants (SNV), indels, gene chimera, and copy number changes).

An exemplary embodiment is a method of making a library of concatenated amplicons from a target nucleic acid, the method comprising:
i. generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons; and
iii. amplifying the one or more concatenated amplicons to generate a library of concatenated amplicons.

Another exemplary embodiment is a library of concatenated amplicons, wherein the library is made by:
i. generating tagged amplicons by amplifying two or more regions of interest (ROIs) from a target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons; and
iii. amplifying the one or more concatenated amplicons to generate a library of concatenated amplicons.

Another exemplary embodiment is a method of selecting a set of primers capable of amplifying two or more regions of interest (ROIs) from a target nucleic acid, comprising selecting a forward primer and a reverse primer for each ROI, wherein each primer comprises a 5′ tag sequence and a sequence capable of hybridizing to the ROI, and wherein:
a) the 5′ tag sequence of the reverse primer for each ROI is complementary to the 5′ tag sequence of the forward primer for another ROI;
b) the 5′ tag sequence is an artificial tag sequence; and
c) each primer comprises minimal sequence that is capable of binding to an ROI and is complementary to a sequence in another primer.

Another exemplary embodiment is a kit comprising a set of primers and instructions for use of the primers in amplifying two or more regions of interest (ROIs) from a target nucleic acid, wherein the set of primers comprises a forward primer and a reverse primer for each ROI, wherein each primer comprises a 5′ tag sequence and a sequence capable of hybridizing to the ROI, and wherein:
a) the 5′ tag sequence of the reverse primer for each ROI is complementary to the 5′ tag sequence of the forward primer for the ROI immediately downstream;
b) the 5′ tag sequence is an artificial tag sequence; and each primer comprises minimal sequence that is capable of hybridizing to an ROI and also complementary to a sequence in another primer.

Also provided herein, in certain aspects, are methods of using the methods and compositions disclosed herein. For instance, in some embodiments, a library of concatenated amplicons (e.g., a library described herein and/or generated using any of the exemplary methods described herein) can be analyzed. In some embodiments, analyzing comprises sequencing, gene assembly, and/or structural variation characterization.

An exemplary embodiment is method of sequencing a library of concatenated amplicons, wherein the library of concatenated amplicons is made by any of the exemplary methods described herein.

Another exemplary embodiment is a method of sequencing a target nucleic acid, the method comprising:
i. generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons;
iii. amplifying the one or more concatenated amplicons to generate a library of concatenated amplicons; and
iv. sequencing the library of concatenated amplicons.

As used herein, the term “region of interest” or “ROI” refers to a nucleic acid (e.g., a genomic sequence, gene, gene fragment, or other nucleic acid of interest) that is analyzed (e.g., using any of the exemplary methods described herein). In some embodiments, an ROI is a portion of a genome or region of genomic DNA. In some embodiments, an ROI comprises or consists of an exon or multiple exons. In some embodiments, an ROI comprises or consists of a portion of an exon. In some embodiments, an ROI comprises more than one ROI. In some embodiments, an ROI may be a template for an amplification reaction (e.g., PCR, e.g., multiplex PCR). In some embodiments, an ROI may be split into two or more amplicons. In some embodiments, amplifying an ROI from a target nucleic acid yields one amplicon (e.g., one tagged amplicon). In some embodiments, amplifying an ROI yields two, 3, 4, or 5, or more, amplicons (e.g., two, 3, 4, or 5, or more, tagged amplicons). In some embodiments, amplifying an ROI yields two amplicons (e.g., two tagged amplicons). In some embodiments, the methods disclosed herein comprise amplifying two or more ROIs from a target nucleic acid. In some embodiments, the methods disclosed herein comprise amplifying at least two, at least 5, at least 10, at least 20, at least 30, at least 40, or at least 50 ROIs from a target nucleic acid. In some embodiments, the methods disclosed herein comprise amplifying at least 2, 3, 4, 5, 6, 7, 8, 9, 10, or more, e.g., at least 12, or at least 14 ROIs from a target nucleic acid.

The term “nucleic acid” is used herein interchangeably with the term “polynucleotide,” and refers to a polymer of nucleotides (e.g., ribonucleotides and deoxyribonucleotides, both natural and non-natural) including DNA, RNA, and their subcategories, such as cDNA, mRNA, etc. A nucleic acid may be single-stranded or double-stranded and generally contains 5-3′ phosphodiester bonds, although in some cases, nucleotide analogs may have other linkages. Nucleic acids may include naturally occurring bases (adenosine, guanosine, cytosine, uracil and thymidine), as well as non-natural bases. Non-natural bases may have a particular function, e.g., increasing the stability of a nucleic acid duplex, inhibiting nuclease digestion, or blocking primer extension or strand polymerization. Unless otherwise indicated, a particular nucleic acid sequence also implicitly encompasses conservatively modified variants thereof (e.g., degenerate codon substitutions) and complementary sequences, as well as the sequence explicitly indicated. In some embodiments, degenerate codon substitutions may be achieved in a nucleic acid by generating sequences in which the third position of one or more selected (or all) codons is substituted with mixed-base and/or deoxyinosine residues (Batzer et al., (1991) Nucleic Acids Res. 25(19):5081; Ohtsuka et al., (1985) J Biol Chem. 260(5):2605-8; Rossolini et al., (1994) Mol Cell Probes 8(2):91-8). In some embodiments, a nucleic acid is a target nucleic acid.

As used herein, the terms “target nucleic acid,” “target sequence,” and “target” are used herein interchangeably to refer to any nucleic acid of interest, or a portion thereof, which is to be amplified, detected, and/or analyzed. The terms also include all variants of a target sequence. In some embodiments, a target nucleic acid is a gene or a gene fragment. In some embodiments, a target nucleic acid is or comprises non-coding sequence(s). In some embodiments, a target nucleic acid is an entire genome, including all genes, gene fragments, and intergenic regions (entire genome). In some embodiments, a target nucleic acid is a portion of a genome, e.g., only the coding regions of a genome (exome). In some embodiments, a target nucleic acid contains a locus of a genetic variant, e.g., a polymorphism, including a single nucleotide polymorphism or variant (SNP or SNV), or a genetic rearrangement resulting, e.g., in a gene fusion. In some embodiments, a target nucleic acid comprises a biomarker, i.e., a gene whose variants are associated with a disease or condition (e.g., a cancer). In some embodiments, a target nucleic acid comprises DNA. The DNA can be, e.g., genomic DNA, mitochondrial DNA, viral DNA, synthetic DNA, or cDNA reverse transcribed from RNA. In some embodiments, the DNA is genomic DNA. In some embodiments, a target nucleic acid is naturally fragmented, e.g., circulating cell-free DNA (cfDNA) or chemically degraded DNA, such as DNA typically found in chemically preserved or archived samples.

The term “amplicon,” as used herein, refers to a nucleic acid generated via an amplification reaction (e.g., PCR or isothermal amplification). An amplicon is typically double-stranded DNA; however, it may be RNA and/or DNA:RNA. In some embodiments, an amplicon comprises DNA complementary to a template nucleic acid (e.g., a target nucleic acid). In some embodiments, one or more primer pairs are selected and/or designed to generate one or more amplicons from a template nucleic acid. As such, in some embodiments, an amplicon comprises the primer pair, the complement of the primer pair, and the region of a template nucleic acid that was amplified to generate the amplicon. In some embodiments, an amplicon further comprises a tag sequence. An amplicon comprising a tag sequence may be referred to herein as a “tagged amplicon.”

As used herein, the term “library” refers to a plurality of nucleic acids. In some embodiments, a library is a library of concatenated amplicons. In some embodiments, a library comprises one or more concatenated amplicons. In some embodiments, a library comprises up to about 200 concatenated amplicons, e.g., about 1 to about 200, about 1 to about 150, about 1 to about 100, about 1 to about 50, about 1 to about 20, or about 1 to about 10 concatenated amplicons. In some embodiments, a library comprises up to about 100 concatenated amplicons, e.g., about 1 to about 100, about 1 to about 50, about 1 to about 20, or about 1 to about 10 concatenated amplicons. In some embodiments, a library comprises up to about 50 concatenated amplicons, e.g., about 1 to about 50, about 1 to about 20, or about 1 to about 10 concatenated amplicons. In some embodiments, a library comprises up to about 20 concatenated amplicons, e.g., about 1, about 5, about 10, about 15, or about 20 concatenated amplicons.

The terms “amplify,” “amplifying,” and “amplification,” as used herein in the context of nucleic acids, refer to the production of one or more copies of a polynucleotide, or a portion of the polynucleotide (e.g., starting from a small amount of the polynucleotide (e.g., a single polynucleotide molecule)), wherein the amplification products or amplicons are generally detectable. Amplification of polynucleotides encompasses a variety of chemical and enzymatic processes. Exemplary forms of amplification include the generation of multiple DNA copies from one or a few copies of a target or template DNA molecule during, e.g., a polymerase chain reaction (PCR) or isothermal amplification. In some embodiments, the amplification reaction is PCR (e.g., multiplex PCR). In some embodiments, the amplification reaction is multiplex PCR. In some embodiments, the amplification reaction is isothermal amplification.

In some embodiments, amplifying two or more ROIs comprises PCR or isothermal amplification. In some embodiments, amplifying two or more ROIs comprises PCR. In some embodiments, amplifying two or more ROIs comprises multiplex PCR.

The term “polymerase chain reaction” or “PCR,” as used herein, refers to a DNA synthesis reaction capable of amplifying a DNA template. A typical PCR reaction mixture comprises primer sequences which are complementary to the ends of a desired template, deoxynucleotide triphosphates (dNTPs), various buffer components, and a DNA polymerase. In general, the reaction mixture is admixed with a DNA sample known or suspected of harboring the desired template. The resulting mixture is then subjected to repeated cycles of template denaturation, primer annealing to the denatured template, and primer extension by the DNA polymerase, to create copies of the template. Because the product of each cycle can act as a template for subsequent reaction cycles, amplification generally proceeds in an exponential fashion (see, e.g., U.S. Pat. No. 4,683,202, and McPherson & Moller, PCR: The Basics (2^{nd} Ed., Taylor & Francisco) (2006)). Variations to this exemplary technique are known in the art and encompassed in the term PCR as used herein.

The term “multiplex PCR,” as used herein, refers to an amplification reaction capable of amplifying multiple DNA templates in parallel (e.g., in a single-tube PCR). In multiplex PCR, more than one target sequence can be amplified, e.g., by using multiple primer pairs in the reaction mixture. Thus, in some embodiments, a plurality of PCR products (i.e., amplicons) can be produced. Multiplex PCR can be broadly divided into single template PCR reactions, and multiple template PCR reactions. A single template PCR reaction may use a single template (e.g., genomic DNA) together with several pairs of forward and reverse primers to amplify specific regions within the template. A multiple template PCR reaction may use multiple templates and several primer sets in the same reaction tube. In some embodiments, multiplex PCR comprises a single template PCR reaction. In some embodiments, multiplex PCR comprises a multiple template reaction. In some embodiments, multiplex PCR is multiplex overlap extension (MOE)-PCR (see, e.g., Kadkhodaei et al., (2016) RSC Adv. 6:66682-94).

In some embodiments, PCR and/or multiplex PCR comprises magnesium, e.g., in a working concentration of about 0.5 mM to about 4 mM. In some embodiments, PCR and/or multiplex PCR comprises magnesium in a working concentration of about 1 mM to about 3.5 mM (e.g., about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, about 3 mM, about 3.1 mM, about 3.2 mM, about 3.3 mM, about 3.4 mM, about 3.5 mM, about 3.6 mM, or about 3.7 mM). In some embodiments, PCR and/or multiplex PCR comprises magnesium in a working concentration of about 1.5 mM to about 3 mM (e.g., about 1.3 mM, about 1.4 mM, about 1.5 mM, about 1.6 mM, about 1.7 mM, about 1.8 mM, about 1.9 mM, about 2 mM, about 2.1 mM, about 2.2 mM, about 2.3 mM, about 2.4 mM, about 2.5 mM, about 2.6 mM, about 2.7 mM, about 2.8 mM, about 2.9 mM, about 3 mM, about 3.1 nM, or about 3.2 nM).

In some embodiments, PCR and/or multiplex PCR comprises dimethyl sulfoxide (DMSO), e.g., in a working concentration of about 1% to about 8% by volume (v/v) (e.g., about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.1%, or about 8.2% by volume). In some embodiments, PCR and/or multiplex PCR comprises DMSO in a working concentration of about 3% to about 6% by volume (e.g., about 2.8%, about 2.9%, about 3%, about 3,1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, or about 6.2% by volume).

In some embodiments, PCR and/or multiplex PCR comprises a pH of about 8 to about 10 (e.g., a pH of about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, about 10, about 10.1, or about 10.2). In some embodiments, PCR and/or multiplex PCR comprises a pH of about 8.5 to about 9.2 (e.g., a pH of about 8.3, about 8.4, about 8.5, about 8.6, about 8.7, about 8.8, about 8.9, about 9, about 9.1, about 9.2, about 9.3, or about 9.4).

The terms “template” and “template nucleic acid” are used herein interchangeably to refer to a nucleic acid that is bound by a primer, e.g., for extension by a nucleic acid synthesis reaction (e.g., by PCR or multiplex PCR). In some embodiments, a nucleic acid synthesis reaction (e.g., PCR or multiplex PCR) uses less than about 2 μg of a template nucleic acid (e.g., template DNA), e.g., less than about 1.9 μg, less than about 1.8 μg, less than about 1.7 μg, less than about 1.6 μg, less than about 1.5 μg, less than about 1.4 μg, less than about 1.3 μg, less than about 1.2 μg, less than about 1.1 μg, or less than about 1.0 μg. In some embodiments, a nucleic acid synthesis reaction (e.g., PCR or multiplex PCR) uses less than about 1 μg of a template nucleic acid (e.g., template DNA), e.g., less than about 0.9 μg, less than about 0.8 μg, less than about 0.7 μg, less than about 0.6 μg, or less than about 0.5 μg.

In some embodiments, amplifying two or more ROIs comprises amplifying at least two, at least 5, at least 10, at least 20, at least 30, at least 40, or at least 50 ROIs. In some embodiments, amplifying two or more ROIs comprises amplifying at least 2, 3, 4, 5, 6, 7, 8, 9, 10, or more, e,g., at least 12, or at least 14 ROIs. In some embodiments, amplifying two or more ROIs comprises amplifying at least two, at least 3, at least 4, at least 5, at least 6, at least 7, at least 8, or at least 9 ROIs. In some embodiments, amplifying two or more ROIs comprises amplifying at least 10, at least 11, at least 12, at least 13, at least 14, at least 15, at least 16, at least 17, at least 18, or at least 19 ROIs. In some embodiments, amplifying two or more ROIs comprises amplifying at least 20, at least 21, at least 22, at least 23, at least 24, at least 25, at least 26, at least 27, at least 28, or at least 29 ROIs. In some embodiments, amplifying two or more ROIs comprises amplifying at least 30, at least 31, at least 32, at least 33, at least 34. at least 35, at least 36, at least 37, at least 38, or at least 39 ROIs. In some embodiments, amplifying two or more ROIs comprises amplifying at least 40, at least 41, at least 42, at least 43, at least 44, at least 45, at least 46, at least 47, at least 48, or at least 49 ROIs. In some embodiments, amplifying two or more ROIs comprises amplifying at least 50 ROIs, or more (e.g., at least 52, at least 55, at least 60, at least 70, at least 80, at least 90, or at least 100 ROIs, or more).

In some embodiments, each ROI is about 2, about 5, about 10, about 20, about 30, about 40, about 50, about 100, about 150, about 200, about 250, about 500, about 1,000, about 2,000, about 5,000, or about 10,000 nucleotides in length. In some embodiments, each ROI is about 2, about 5, about 10, about 20, about 30, about 40 nucleotides in length. In some embodiments, each ROI is about 50, about 60, about 70, about 80, or about 90 nucleotides in length. In some embodiments, each ROI is about 100, about 110, about 120, about 130, or about 140 nucleotides in length. In some embodiments, each ROI is about 150, about 160, about 170, about 180, or about 190 nucleotides in length. In some embodiments, each ROI is about 200, about 210, about 220, about 230, or about 240 nucleotides in length. In some embodiments, each ROI is about 250, about 300, about 350, about 400, or about 450 nucleotides in length. In some embodiments, each ROI is about 500, about 550, about 600, about 650, about 700, about 750, about 800, about 850, about 900, or about 950 nucleotides in length. In some embodiments, each ROI is about 1,000, about 1,100, about 1,200, about 1,300, about 1,400, about 1,500, about 1,600, about 1,700, about 1,800, or about 1,900 nucleotides in length. In some embodiments, each ROI is about 2,000, about 2,200, about 2,400, about 2,600, about 2,800, about 3,000, about 3,200, about 3,400, about 3,600, about 3,800, about 4,000, about 4,200, about 4,400, about 4,600, or about 4,800 nucleotides in length. In some embodiments, each ROI is about 5,000, about 5,500, about 6,000, about 6,500, about 7,000, about 7,500, about 8,000, about 8,500, about 9,000, or about 9,500 nucleotides in length. In some embodiments, each ROI is about 10,000 nucleotides in length, or more (e.g., about 12,000, about 15,000, or about 20 nucleotides in length, or more),

The term “primer,” as used herein, refers to a polynucleotide capable of hybridizing with a sequence in a target nucleic acid (e.g., an ROI) and acting as a point of initiation of synthesis for a complementary strand of a nucleic acid under conditions suitable for such synthesis (e.g., in the presence of nucleotides and an inducing agent such as a DNA polymerase and at a suitable temperature and pH). In some embodiments, a primer is single-stranded for maximum efficiency in amplification, but may alternatively be double-stranded. If double-stranded, in some embodiments, the primer is first treated to separate its strands before being used to prepare extension products. In some embodiments, the primer is DNA. In some embodiments, the primer is sufficiently long to prime the synthesis of extension products in the presence of an inducing agent (e.g., a DNA polymerase). The exact lengths of primers may depend on several factors, including temperature, source of primer, and the use of the method, as will be apparent to one of skill in the art. In some embodiments, a primer is about 18-22 nucleotides in length. In some embodiments, a primer is about 16, about 17, about 18, about 19, about 20, about 21, about 22, about 23, or about 24 nucleotides in length. In some embodiments, a primer is less than about 18 nucleotides in length. In some embodiments, a primer is greater than about 22 nucleotides in length. In some embodiments, a primer comprises at least one sequence or sequence portion that does not hybridize to the nucleic acid of interest. For example, in some embodiments, a primer may comprise a tag sequence (e.g., any of the tag sequences described and/or exemplified herein). In some embodiments, a primer is a forward primer. In some embodiments, a primer is a reverse primer. In some embodiments, a primer comprises a set of primers (e.g., at least one forward primer and at least one reverse primer).

The term “forward primer,” as used herein, refers to a primer capable of annealing to a 5′ end of a template. In some embodiments, a forward primer can anneal to about 15-30, about 15-25, about 15-20, about 20-30, or about 20-25 nucleotides at a 5′ end of the template.

The term “reverse primer,” as used herein, refers to a primer capable of annealing to a 3′ end of a template (e.g., to a 5′ end of a reverse strand of the template). In some embodiments, a reverse primer can anneal to about 15-30, about 15-25, about 15-20, about 20-30, or about 20-25 nucleotides at a 3′ end of the template.

In some embodiments, the working concentration of one or more primers is about 1 nM to about 5,000 nM. In some embodiments, the working concentration of one or more primers is about 5 nM, about 10 nM, about 20 nM, about 30 nM, about 40 nM, about 50 nM, about 60 nM, about 70 nM, about 80 nM, about 90 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1,000 nM. In some embodiments, the working concentration of one or more primers is about 1,000 nM, about 1,250 nM, 1,500 nM, about 1,750 nM, about 2,000 nM, about 2,250 nM, about 2,500 nM, about 2,750 nM, about 3,000 nM, about 3,250 nM, about 3,500 nM, about 3,750 nM, about 4,000 nM, about 4,250 nM, about 4,500 nM, about 4,750 nM, or about 5,000 nM, or higher. In some embodiments, the working concentration of one or more primers is about 10 nM to about 100 nM. In some embodiments, the working concentration of one or more primers is about 10 nM to about 50 nM. In some embodiments, the working concentration of one or more primers is about 20 nM to about 40 nM. In some embodiments, the working concentration of one or more primers is about 30 nM.

In some embodiments, one or more primers are depleted prior to concatenating tagged amplicons. The term “depleted” or “depletion,” as used herein in the context of primer concentration, means reducing a primer concentration by at least about 50%, at least about 60%, at least about 70%, at least about 80%, at least about 90%, at least about 95%, or at least about 99%, or 100%, relative to the starting concentration of the primer (i.e., 100% depletion is not necessarily achieved). In some embodiments, a primer concentration is reduced or depleted by at least about 80%, at least about 90%, at least about 95%, or at least about 99%. In some embodiments, a primer concentration is reduced or depleted by 100%.

In some embodiments, one or more primers are selected to prevent formation of one or more primer dimers.

As used herein, the term “primer dimer” refers to a nucleic acid molecule comprising or consisting of at least two primers that have attached (i.e., hybridized) to each other due to strings of complementary bases in the primers. Primer dimers can be a potential by-product in amplification reactions such as PCR. In some embodiments, a DNA polymerase may amplify one or more primer dimers, which can result in competition for reagents and potentially inhibit amplification of the DNA sequence targeted for amplification. In some embodiments, a primer dimer may result in skipping of amplicons and/or generation of truncated amplification products. In some embodiments, such as in quantitative PCR, primer dimers may interfere with accurate quantification. In some embodiments, the methods and compositions described herein comprise selecting one or more primers that lack 5 or more (e.g., 5, 6, 7, 8, 9, 10, or more) exactly-matched bases (i.e., exactly-matched bases with one another or with any other primers) at the 3′ end of the primer sequences. In some embodiments, such selection may prevent two primers from forming a primer dimer (e.g., an exponential amplifiable primer dimer). In some embodiments, such selection may prevent two primers from forming a primer dimer (e.g., a linear amplifiable primer dimer). In some embodiments, such selection may prevent two primers from forming one or more non-specific off-target products. In some embodiments, one or more primers are selected to comprise minimal sequence that is complementary to a sequence in another primer used in generating a nucleic acid library. In some embodiments, the minimal sequence is about 6 to about 100 nucleotides in length, e.g., about 6 to about 50 or about 15 to about 30 nucleotides in length, e.g., about 18 to about 20 nucleotides in length. In some embodiments, the minimal sequence is about 6 to about 50 nucleotides in length, e.g., about 6 to about 30 or about 15 to about 30 nucleotides in length, e.g., about 18 to about 20 nucleotides in length. In some embodiments, the minimal sequence is about 6 to about 30 nucleotides in length. In some embodiments, the minimal sequence is about 4 to about 40, about 5 to about 35, or about 6 to about 30 nucleotides in length. In some embodiments, the minimal sequence is about 10, about 15, about 20, about 25, about 30, or about 35 nucleotides in length. In some embodiments, the minimal sequence is about 15 to about 30 nucleotides in length. In some embodiments, the minimal sequence is about 18 to about 20 nucleotides in length. In some embodiments, the minimal sequence is at least about 4, about 5, about 6, about 7, about 8, about 9, or about 10 nucleotides in length. In some embodiments, the minimal sequence is at least about 6 nucleotides in length.

In some embodiments, one or more primers are selected to minimize formation of one or more dead-end intermediate products. In some embodiments, one or more primers comprise a 5′ tag sequence and a sequence capable of hybridizing to an ROI. In some embodiments, the methods and compositions described herein comprise selecting one or more primers that have at least one adenine between the 5′ tag sequence and the sequence capable of hybridizing to an ROI. In some embodiments, such selection may minimize or eliminate formation of one or more dead-end intermediate products.

As used herein, the term “dead-end intermediate product” refers to a nucleic acid molecule produced in an amplification reaction (e.g., PCR) that cannot form one or more concatenated amplicons.

As used herein, the term “tag sequence” refers to a nucleic acid that is not capable of hybridizing with a sequence in a target nucleic acid (e.g., an ROI). In some embodiments, a tag sequence may be about 10-60 nucleotides in length. In some embodiments, a tag sequence is about 8, about 9, about 10, about 11, about 12, about 13, about 14, about 15, about 16, about 17, about 18, about 19, about 20, about 21, about 22, about 23, about 24, about 25, about 26, about 27, about 28, or about 29 nucleotides in length. In some embodiments, a tag sequence is about 30, about 35, about 40, about 45, about 50, about 55, or about 60 nucleotides in length, or longer (e.g., about 65 or about 70 nucleotides in length, or longer). In some embodiments, a tag sequence of a primer or amplicon is complementary to a tag sequence of another primer or amplicon. In some embodiments, a tag sequence serves as a template for concatenation. For example, in some embodiments, a 5′ tag sequence of a reverse primer for an ROI is complementary to a 5′ tag sequence of a forward primer for another ROI. In some embodiments, following amplification, the tag sequences in the resulting amplicons may hybridize and allow concatenation of the tagged amplicons. In some embodiments, a tag sequence in one or more primers and/or in one or more amplicons is an artificial tag sequence. The term “artificial” refers to a sequence that is not homologous to any part of a genomic sequence (e.g., a human genome sequence).

Two sequences are “not homologous” if two sequences have a low percentage of nucleotides that are the same (e.g., less than about 70% identity over a specified region, or, when not specified, over the entire sequence), e.g., when compared and aligned for maximum correspondence over a comparison window, or designated region as measured using a sequence comparison algorithm or by manual alignment and visual inspection. Optionally, the identity exists over a region that is at least about 50 nucleotides (or 10 amino acids) in length, or over a region that is 100 to 500 or 1000 or more nucleotides (or 20, 50, 200 or more amino acids) in length. In some embodiments, the identity exists over a region that is at least about 15, 20, 25, 30, 35, 40, 45, or 50 nucleotides in length. In some embodiments, the identity exists over a region that is at least about 20 nucleotides in length.

In some embodiments, a tag sequence in one or more primers and/or in one or more amplicons is an artificial tag sequence that is less than about 70% identical to any part of a genomic sequence (e.g., a human genomic sequence). In some embodiments, a tag sequence in one or more primers and/or in one or more amplicons is an artificial tag sequence that is less than about 60% identical to any part of a genomic sequence (e.g., a human genomic sequence). In some embodiments, a tag sequence in one or more primers and/or in one or more amplicons is an artificial tag sequence that is less than about 50% identical to any part of a genomic sequence, or less (e.g., a human genomic sequence). In some embodiments, percent (%) identity between an artificial tag sequence and a genomic sequence (e.g., a human genomic sequence) is measured over the entire length of the artificial tag sequence.

The percent “identity” between two sequences is a function of the number of identical positions shared by the sequences (i.e., percent identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Additionally, or alternatively, the sequences of the present disclosure can further be used as a “query sequence” to perform a search against public databases to, for example, identify related sequences. For example, such searches can be performed using the BLAST program of Altschul et al. (J Mol Biol 1990; 215(3):403-10).

In some embodiments, an artificial tag sequence is about 20 nucleotides in length, or longer (e.g., about 25 or about 30 nucleotides in length, or longer). In some embodiments, an artificial tag sequence is about 20 nucleotides in length, or longer (e.g., about 25 or about 30 nucleotides in length, or longer), and percent (%) identity between the artificial tag sequence and a genomic sequence (e.g., a human genomic sequence) is measured over the entire length of the tag. In some embodiments, an artificial tag sequence is a 5′ tag sequence, e.g., a tag sequence at the 5′ end of a primer or amplicon. In some embodiments, an artificial tag sequence is a 5′ tag sequence that can be used in an amplification reaction without interference from a sequence in a target nucleic acid (e.g., a human genomic sequence).

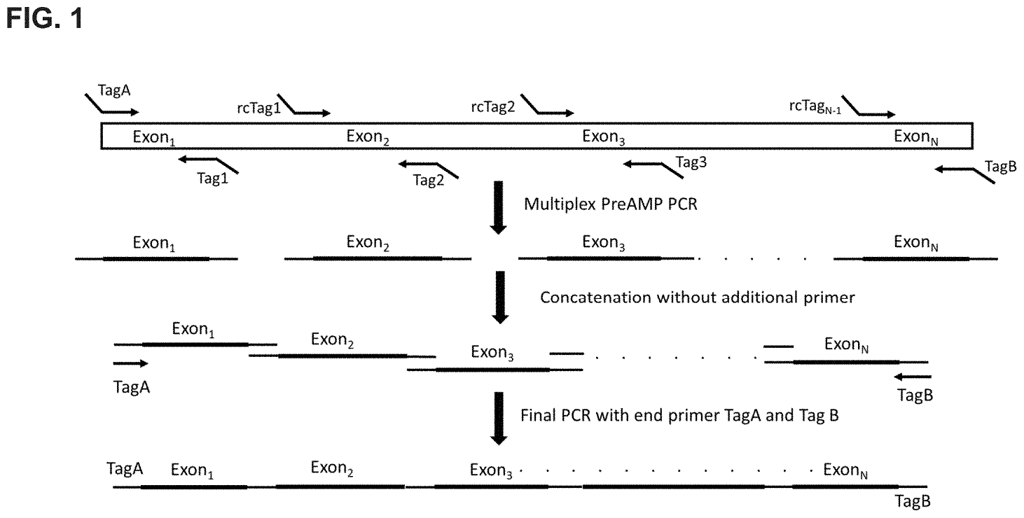

In some embodiments, tagged, sequence-specific primers are designed such that the 5′ tag sequence of the reverse primer for each ROI is complementary to the 5′ tag sequence of the forward primer for another ROI. In some embodiments, tagged, sequence-specific primers are designed such that the 5′ tag sequence of the reverse primer for each ROI is complementary to the 5′ tag sequence of the forward primer for the ROI immediately downstream. For instance, in some embodiments, tagged, sequence-specific primers are designed as shown in FIG. 1 for a particular target nucleic acid of interest (i.e., a 5′ Tag₁ of reverse primer of Exon₁ is designed to be complementary to a 5′ rcTag₁ of forward primer of Exon₂, a 5′ Tag₂ of reverse primer of Exon₂ is designed to be complementary to a 5′ rcTag₂ of forward primer of Exon₃, etc.). Exemplary tags and primers are described and exemplified herein.

In some embodiments, one or more primers comprise at least one adenine between the 5′ tag sequence and the sequence capable of hybridizing to the ROI. In some embodiments, one or more primers comprise a 5′ phosphate. In some embodiments, use of phosphorylated primers may improve specificity of amplicon ligation and concatenation (e.g., following PCR (e,g., following multiplex PCR)).

In some embodiments, one or more primers comprise a molecular barcode. The term “barcode” refers to a nucleic acid sequence that can be detected and identified, e.g., to track, categorize, or index amplified samples. Barcodes can be incorporated into various nucleic acids. Barcodes can also be sufficiently long (e.g., at least 6, 10, or 20 nucleotides in length) such that nucleic acids incorporating the barcodes can be distinguished or grouped according to the barcodes. In some embodiments, a barcode is at least 6 nucleotides in length (e.g., about 6, about 7, about 8, or about 9 nucleotides in length, or longer). In some embodiments, a barcode is at least 10 nucleotides in length (e.g., about 10, about 11, about 12, about 13, about 14, about 15, about 16, about 17, about 18, or about 19 nucleotides in length, or longer). In some embodiments, a barcode is at least 20 nucleotides in length, or longer. Exemplary barcodes and uses thereof are described in U.S. Pat. No. 8,318,434, which is incorporated herein by reference.

In some embodiments, barcodes may be used to quantify the original copy input of each ROI. In some embodiments, the copy input information allows detection of copy number variation. A tag sequence may comprise a barcode. In some embodiments, one or more primers comprise a barcode within a tag sequence (e.g., a 5′ tag sequence). In some embodiments, a barcode included within a tag sequence (e.g., a 5′ tag sequence) can label each individual target molecule (e.g., each tagged amplicon) with a unique barcode sequence. For instance, in some embodiments, an amplification reaction using 10 ng input of human genomic DNA may yield approximately 3000 unique copies of a particular gene, with each copy labeled with a unique barcode. By counting the number of unique barcodes in the final sequencing reads, in some embodiments, the copy number of input molecules can be determined. For example, in some embodiments, a two-copy gene having twice the number of starting copies for amplification may have twice the number of unique barcode counts, as compared to a one-copy gene. In some embodiments, the number of unique barcode sequences incorporated into a concatemer can be counted and compared to reference counts for a known copy-number gene. In some embodiments, the copy number of the target gene can be calculated based on the molecular barcode counting ratio relative to the reference gene.

In some embodiments, each tagged amplicon is labeled with a unique barcode sequence, and the barcodes are used to determine the copy number of each amplicon target in the starting input. In some embodiments, following amplification, concatenation, and sequencing, each amplicon having the same stoichiometry ratio (e.g., a stoichiometry ratio of about 1:1, i.e., one amplicon to one concatemer) can result in the same total reads for each amplicon. In some embodiments, if each tagged amplicon is labeled with a unique barcode sequence, barcode counting can also simultaneously allow for quantification of the actual copy number of each target amplicon in the starting input. In some embodiments, a purification step is used to remove any unincorporated barcode primers from the reaction mixture following amplification. In some embodiments, if excess barcode primers are not removed (e.g., via purification), a resampling of PCR products may occur (e.g., during a subsequent amplification reaction (e.g., during a subsequent PCR)) and result in falsely high numbers of unique copies of a target amplicon, e.g., as determined by sequencing analysis. Exemplary methods for copy number detection using barcodes are described in Ogawa et al., (2017) Scientific Reports 7(1):13576, which is incorporated herein by reference for such methods.

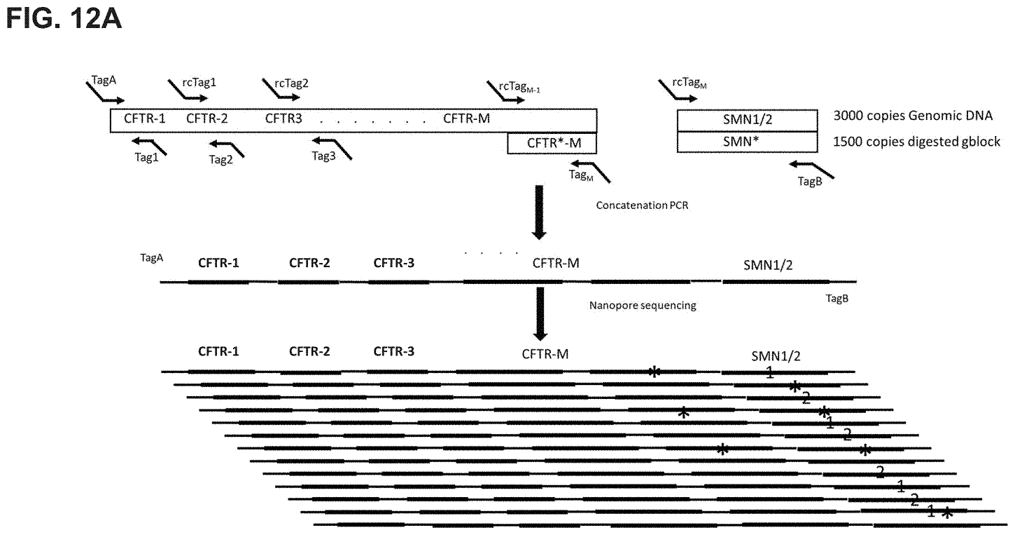

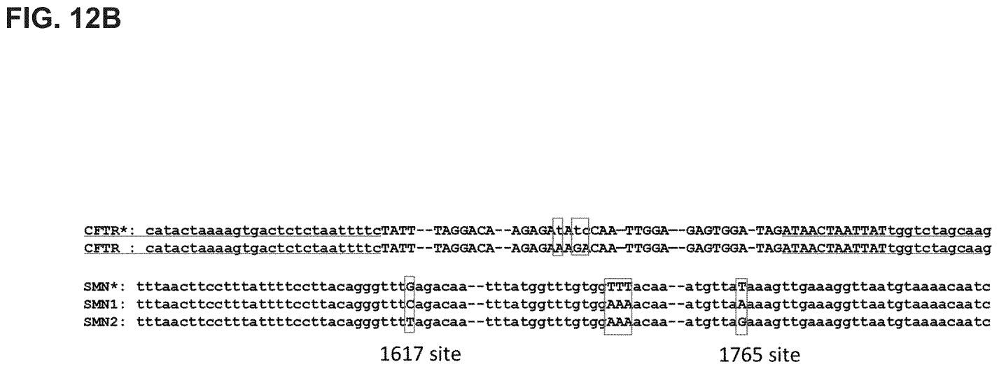

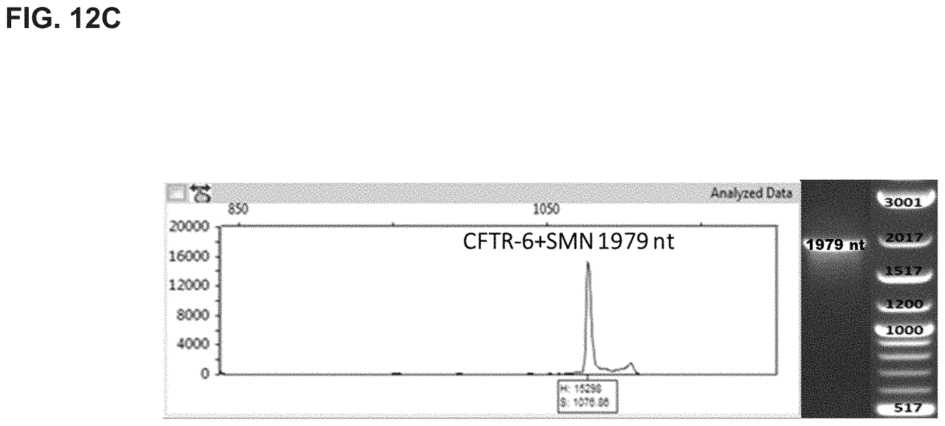

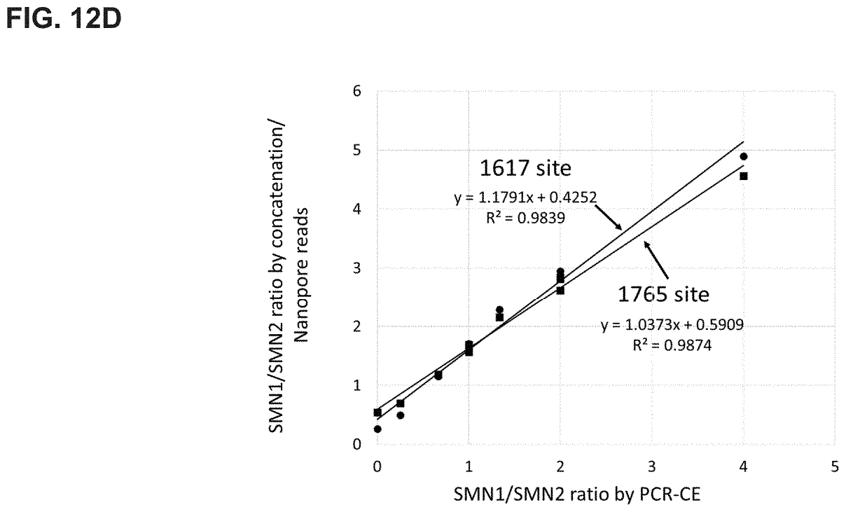

In some embodiments, an external spiking control may be used to quantify the original copy input of each ROI. In some embodiments, detecting or quantifying gene copy number comprises using and/or comparing to an external spiking control. In some embodiments, the external spiking control is added during amplification of two or more ROIs, e.g., in step (i) of a multiplex PCR. In some embodiments, the external spiking control comprises a spiking synthetic gBlock control. In some embodiments, the external spiking control (e.g., a spiking synthetic gBlock control) comprises gene fragments of a reference gene with a known copy number and a target gene with an unknown copy number. In some embodiments, each synthetic gene fragment contains at least one stamp code, e.g., a different base compared to the natural genomic sequence, which allows for differentiation between the natural genomic sequences and the artificial synthetic gBlocks. In some embodiments, two or more gene fragments are constructed in one synthetic gBlock to maintain a 1:1 stoichiometry ratio. In some embodiments, two or more gene fragments in a synthetic gBlock may have the opposite 5′-3′ orientation as the orientation in the final concatenation products. In some embodiments, a unique restriction site is used to cut the synthetic gBlock while maintaining an equal (1:1) molar ratio of the two or more gene fragments in the digested gBlock control. Exemplary methods for copy number detection using an external spiking control (e.g., a spiking synthetic gBlock control) are described and exemplified herein (e.g., in Example 7 and FIG. 12A-12D).

The terms “concatenate,” “concatenating,” and “concatenation,” as used herein, refer to the linkage (e.g., covalent linkage) of two or more nucleic acids (e.g., amplicons, e.g., tagged amplicons). The terms “concatemer” and “concatenated amplicon” refer to a continuous nucleic acid molecule generated by linking (e.g., covalently linking) shorter nucleic acid molecules such as amplicons (e.g., tagged amplicons).

In some embodiments, tagged amplicons are not purified prior to concatenation. In some embodiments, tagged amplicons are joined to form one or more concatenated amplicons. In some embodiments, concatenating the tagged amplicons comprises concatenating at least two, at least 5, at least 10, at least 20, at least 30, at least 40, or at least 50 tagged amplicons. In some embodiments, concatenating the tagged amplicons comprises concatenating at least two, at least 3, at least 4, at least 5, at least 6, at least 7, at least 8, or at least 9 tagged amplicons. In some embodiments, concatenating the tagged amplicons comprises concatenating at least 10, at least 11, at least 12, at least 13, at least 14, at least 15, at least 16, at least 17, at least 18, or at least 19 tagged amplicons. In some embodiments, concatenating the tagged amplicons comprises concatenating at least 20, at least 21, at least 22, at least 23, at least 24, at least 25, at least 26, at least 27, at least 28, or at least 29 tagged amplicons. In some embodiments, concatenating the tagged amplicons comprises concatenating at least 30, at least 31, at least 32, at least 33, at least 34, at least 35, at least 36, at least 37, at least 38, or at least 39 tagged amplicons. In some embodiments, concatenating the tagged amplicons comprises concatenating at least 40, at least 41, at least 42, at least 43, at least 44, at least 45, at least 46, at least 47, at least 48, or at least 49 tagged amplicons. In some embodiments, concatenating the tagged amplicons comprises concatenating at least 50 tagged amplicons, or more (e.g., at least 52, at least 55, at least 60, at least 70, at least 80, at least 90, or at least 100 tagged amplicons, or more).

In some embodiments, each tagged amplicon is about 50, about 100, about 150, about 200, about 250, about 500, about 1,000, about 2,000, about 5,000, or about 10,000 nucleotides in length. In some embodiments, each tagged amplicon is about 50, about 60, about 70, about 80, or about 90 nucleotides in length. In some embodiments, each tagged amplicon is about 100, about 110, about 120, about 130, or about 140 nucleotides in length. In some embodiments, each tagged amplicon is about 150, about 160, about 170, about 180, or about 190 nucleotides in length. In some embodiments, each tagged amplicon is about 200, about 210, about 220, about 230, or about 240 nucleotides in length. In some embodiments, each tagged amplicon is about 250, about 300, about 350, about 400, or about 450 nucleotides in length. In some embodiments, each tagged amplicon is about 500, about 550, about 600, about 650, about 700, about 750, about 800, about 850, about 900, or about 950 nucleotides in length. In some embodiments, each tagged amplicon is about 1,000, about 1,100, about 1,200, about 1,300, about 1,400, about 1,500, about 1,600, about 1,700, about 1,800, or about 1,900 nucleotides in length. In some embodiments, each tagged amplicon is about 2,000, about 2,200, about 2,400, about 2,600, about 2,800, about 3,000, about 3,200, about 3,400, about 3,600, about 3,800, about 4,000, about 4,200, about 4,400, about 4,600, or about 4,800 nucleotides in length. In some embodiments, each tagged amplicon is about 5,000, about 5,500, about 6,000, about 6,500, about 7,000, about 7,500, about 8,000, about 8,500, about 9,000, or about 9,500 nucleotides in length. In some embodiments, each tagged amplicon is about 10,000 nucleotides in length, or more (e.g., about 12,000, about 15,000, or about 20 nucleotides in length, or more).

In some embodiments, the total length of the one or more concatenated amplicons is about 2,000 to about 50,000 nucleotides. In some embodiments, the total length of the one or more concatenated amplicons is about 2,000 to about 20,000 nucleotides. In some embodiments, the total length of the one or more concatenated amplicons is about 10,000 nucleotides. In some embodiments, the total length of the one or more concatenated amplicons is about 5,000 nucleotides. In some embodiments, the total length of the one or more concatenated amplicons is about 3,000 to about 4,000 nucleotides. In some embodiments, concatenating tagged amplicons to generate one or more concatenated amplicons allows each amplicon to have a desired orientation. In some embodiments, concatenating involves hybridization of the complementary ends (i.e., tags) of the tagged amplicons.

The terms “hybridize,” “hybridizing,” and “hybridization,” as used herein, refer to the formation of a complex between nucleotide sequences that are sufficiently complementary to form a complex via Watson-Crick base pairing. For example, in some embodiments, where a primer “hybridizes” with target (template) nucleic acid, the complex (hybrid) is sufficiently stable to serve the priming function required by, e.g., the DNA polymerase to initiate DNA synthesis. In some embodiments, where the complementary end (i.e., tag) of a tagged amplicon “hybridizes” with the complementary end (i.e., tag) of another tagged amplicon, the complex is sufficiently stable to form a concatamer of the tagged amplicons. In some embodiments, wherein a primer comprises a sequence capable of hybridizing to an ROI, the sequence in the primer and the ROI may be, but are not necessarily, completely complementary. In some embodiments, the sequence in the primer and the ROI have a perfectly matched stretch of bases that is capable of forming a complex via Watson-Crick base pairing (i.e., is 100% complementary). In some embodiments, the sequence in the primer and the ROI do not have a perfectly matched stretch of bases, but are sufficiently complementary to form a complex via Watson-Crick base pairing (e.g., the sequence in the primer and the ROI are at least about 80%, 85%, 90%, 95%, or 99% complementary).

The term “complementary,” as used herein in connection with a nucleic acid sequence, refers to the pairing of bases, A with T or U, and G with C. The term can refer to nucleic acid molecules that are completely complementary (i.e., capable of forming A to T or U pairs and G to C pairs across the entire reference sequence), as well as molecules that are substantially complementary (e.g., at least about 80%, 85%, 90%, 95%, or 99% complementary).

In some embodiments, one or more concatenated amplicons are in a predetermined order. In some embodiments, the predetermined order results from the tag sequences in the primers. In some embodiments, the 5′ tag sequence of the reverse primer for each ROI is complementary to only the 5′ tag sequence of the forward primer for the ROI immediately downstream. In some embodiments, the order of the one or more concatenated amplicons is identical to the order of the corresponding ROIs in the target nucleic acid. In some embodiments, the order of the one or more concatenated amplicons is not identical to the order of the corresponding ROIs in the target nucleic acid and is driven instead by the predetermined pairing of the 5′ tag sequence of the reverse primer of each ROI with the 5′ tag sequence of the forward primer of another ROI. In some embodiments, the one or more concatenated amplicons comprise single-copy representation (e.g., a defined unitary copy number) of each tagged amplicon. As used herein, the term “single-copy representation” means that a concatenated amplicon contains a single copy of each tagged amplicon used to assemble the concatenated amplicon. In some embodiments, the ratio of the one or more concatenated amplicons to the corresponding ROIs in the target nucleic acid is about 1 to 1. Other ratios (i.e., any ratios other than about 1 to 1) are also contemplated and may result from the exemplary methods and compositions disclosed herein.

In some embodiments, concatenating tagged amplicons comprises providing a DNA polymerase. In some embodiments, the DNA polymerase fills in the gaps in the structures formed by hybridization of the complementary ends (i.e., tags) of the tagged amplicons. In some embodiments, the DNA polymerase is a wild-type polymerase. In some embodiments, the DNA polymerase is a modified polymerase. In some embodiments, the DNA polymerase is a thermophilic, chimeric, and/or engineered polymerase. In some embodiments, the DNA polymerase can comprise a mixture of more than one polymerase. In some embodiments, the DNA polymerase has 3′ to 5′ exonuclease activity. In some embodiments, the DNA polymerase is a high-fidelity DNA polymerase. In some embodiments, the DNA polymerase is a Q5, Pfu, or Kapa HiFi HotStart DNA polymerase.

In some embodiments, the DNA polymerase is a Q5 DNA polymerase, e,g., M0494S, M0491S (New England Biolabs Inc.) (see, e.g., U.S. Pat. Nos. 6,627,424, 7,541,170, 7,670,808, and 7,666,645, each of which is incorporated herein by reference for the description of such polymerases and uses thereof).

In some embodiments, the DNA polymerase is a Pfu DNA polymerase, e.g., M7741/M7745 (Promega) (see, e.g., Mesalam et al., (2018) Virology 514:30-41; Pasello et al., (2018) Methods in Molecular Biology 1827; Harvey et al., (2018) Journal of Chemical Ecology 44(10):894-904; Dubos et al., (2018) General and Comparative Endocrinology 266:110-118; and Tanabe et al., (2018) Revista do Instituto de Medicina Tropical de São Paulo 60, each of which is incorporated herein by reference for the description of such polymerases and uses thereof).

In some embodiments, the DNA polymerase is a Kapa HiFi HotStart DNA polymerase, e.g., KK2601/KK2602 (Roche) (see, e.g., U.S. Pat. No. 8,481,685, which is incorporated herein by reference for the description of such polymerases and uses thereof).

In some embodiments, concatenating tagged amplicons comprises providing at least one adjuvant. The term “adjuvant,” as used herein, refers to a reagent capable of improving efficiency (i.e., higher amount of product) and/or specificity (i.e., lower amount of non-specific product) of an amplification reaction (e.g., PCR, e.g., multiplex PCR). In some embodiments, the at least one adjuvant comprises TMAC, ThermaGo, and/or ThermaStop. In some embodiments, the at least one adjuvant comprises trioctadecylmethylammonium chloride (TMAC). In some embodiments, the at least one adjuvant comprises ThermaGo (ThermaGo™ (Thermagenix)). In some embodiments, the at least one adjuvant comprises ThermaStop (ThermaStop™ (Thermagenix)). See, e.g., U.S. Pat. Nos. 7,517,977, 9,034,605, and 9,758,813; see also U.S. Publication No. 201810002739, each of which is incorporated herein by reference for the description of such adjuvants.

In some embodiments, amplifying the one or more concatenated amplicons comprises PCR. In some embodiments, amplifying the one or more concatenated amplicons comprises long-range PCR (i.e., PCR capable of amplifying templates at least about 10,000 nucleotides in length, or longer). Exemplary protocols, including reagents and reaction conditions, for long-range PCR are described in, e.g., Cheng et al., (1994) PNAS 91:5695-9; Barnes (1994) PNAS 91(6):2216-20; and Jia et al., (2014) Scientific Reports 4:5737, each of which is incorporated herein by reference for the disclosure of such protocols.

In some embodiments, amplifying the one or more concatenated amplicons comprises at least one first end primer and at least one second end primer.

As used herein, the term “end primer” refers to a primer capable of hybridizing with a tag sequence at an end (i.e., a 5′ or 3′ end) of a concatenated amplicon. In some embodiments, an end primer acts as a point of initiation of synthesis along a complementary strand of the concatenated amplicon. In some embodiments, the end primer is used to amplify the concatenated amplicon. In some embodiments, an end primer comprises a first end primer and a second end primer. In some embodiments, the first end primer is capable of hybridizing to a tag sequence at the 5′ end of a concatenated amplicon. In some embodiments, the 5′ end of the concatenated amplicon is identical to or overlaps with the 5′ tag sequence of a forward primer used to amplify an ROI. In some embodiments, the second end primer is capable of hybridizing to a tag sequence at the 3′ end of a concatenated amplicon. In some embodiments, the tag sequence at the 3′ end of the concatenated amplicon is identical to or overlaps with the 5′ tag sequence of a reverse primer used to amplify an ROI. Exemplary end primers are described and exemplified herein. Exemplary end primers, and their use in an exemplary method disclosed herein, are also shown in FIG. 1 (TagA and TagB primers).

In some embodiments, a first end primer and a second end primer are added during generation of tagged amplicons, concatenation of tagged amplicons, or amplification of one or more concatenated amplicons (i.e., in any one of steps (i)-(iii), respectively). In some embodiments, a first end primer and a second end primer are added in step (ii) or step (iii). In some embodiments, a method disclosed herein comprises 2-step PCR.

As used herein, the term “2-step PCR” refers to a method comprising a first PCR and a second PCR. In some embodiments, the first PCR and the second PCR are carried out without an intervening purification step (i.e., a purification step between the first and second PCR). In some embodiments, the first PCR comprises multiplex PCR. In some embodiments, the first PCR comprises the protocol: 94° C./5 min, 2 cycles of 94° C./15 sec, 60° C./4 min, and 23 cycles of 94° C./15 sec, 72° C./2 min, followed by 20 cycles of 94° C./15 sec, 55° C./1 min, 72° C./2 min. In some embodiments, the second PCR comprises amplification of the products from the first PCR (e.g., about 1 μl of PCR products) with end primers. In some embodiments, the end primers are added before or during the second PCR. In some embodiments, 2-step PCR may be performed in less than about 5 hours, less than about 4.5 hours, less than about 4 hours, less than about 3.5 hours, or less than about 3 hours. In some embodiments, 2-step PCR may be performed in less than about 4 hours. In some embodiments, the total active (“hands-on”) time of 2-step PCR may be less than about 1 hour, less than about 50 min, less than about 40 min, less than about 30 min, or less than about 20 min. In some embodiments, the total active time of 2-step PCR may be less than about 30 min.

In some embodiments, a first end primer and a second end primer are added in step (i). In some embodiments, a method disclosed herein comprises 1-step PCR.

As used herein, the term “1-step PCR” refers to a method comprising a single PCR. In some embodiments, the single PCR comprises PCR and amplification of the products from the PCR (e.g., about 1 μl of PCR products) with end primers. In some embodiments, the PCR comprises multiplex PCR.

In some embodiments, a target nucleic acid is obtained from a biological sample (e.g., a biological sample from a human subject diagnosed with and/or suspected of being at risk for a disease (e.g., a cancer or a hereditary disorder)). In some embodiments, a target nucleic acid is used in a multiple gene panel, e.g., to detect mutations and/or structural variation in one or more target genes. In some embodiments, the multiple gene panel is a newborn or carrier screening panel. In some embodiments, the multiple gene panel comprises at least about 20 human genes (e.g., at least about 22 human genes). In some embodiments, the multiple gene panel comprises at least about 22 human genes.

In some embodiments, a library of concatenated amplicons is made from the target nucleic acid, e.g., using any of the exemplary methods disclosed herein. For example, in some embodiments, a library of concatenated amplicons is made by generating tagged amplicons from the target nucleic acid (e.g., by amplifying two or more regions of interest (ROIs)); concatenating the tagged amplicons to generate one or more concatenated amplicons; and amplifying the one or more concatenated amplicons to generate the library.

In some embodiments, two or more ROIs (e.g., ROIs in exon regions) are amplified (e.g., by PCR, e.g., by multiplex PCR) with gene-specific primers each having a tag sequence attached to the 5′ end of the primer. In some embodiments, two or more ROIs are amplified by multiplex PCR (e.g., MOE-PCR). In some embodiments, each ROI is amplified with a forward primer and a reverse primer. In some embodiments, each primer comprises a 5′ tag sequence and a sequence capable of hybridizing to an ROI. In some embodiments, the 5′ tag sequence of the reverse primer for each ROI is complementary to the 5′ tag sequence of the forward primer for another ROI. In FIG. 1, for example, the 5′ Tag₁ of reverse primer of Exon₁ is designed to be complementary to the 5′ rcTag₁ of forward primer of Exon₂, etc. Following amplification, in some embodiments, the amplicons comprise complementary tag sequences, which allow the tagged amplicons to be assembled into a single concatenated product. In some embodiments, end primers with tag sequences may be used to drive amplification of the concatenated product and generate an integrated long template (e.g., a template for sequencing (e.g., single-molecule sequencing)). In some embodiments, a first end primer is capable of hybridizing to a tag sequence at the 5′ end of a concatenated amplicon. In some embodiments, a second end primer is capable of hybridizing to a tag sequence at the 3′ end of a concatenated amplicon. Exemplary end primers include, without limitation, TagA and TagB primers in FIG. 1.

In some embodiments, the library of concatenated amplicons made from the target nucleic acid is analyzed. In some embodiments, the library is analyzed using sequencing (e.g., single-molecule sequencing), gene assembly, and/or structural variation characterization. In some embodiments, the library is sequenced, e.g., using single-molecule sequencing or any long-read sequencing platform.

In some embodiments, the present disclosure provides method of sequencing a target nucleic acid, the method comprising:
i. providing a target nucleic acid from a biological sample;
ii. generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
iii. concatenating the tagged amplicons to generate one or more concatenated amplicons, wherein the one or more concatenated amplicons are in a predetermined order and comprise single-copy representation of each tagged amplicon;
iv. amplifying the one or more concatenated amplicons to generate a library of concatenated amplicons; and
v. sequencing the library of concatenated amplicons.

In some embodiments, the target nucleic acid is isolated from a biological sample. In some embodiments, the biological sample is obtained from a subject (e.g., a human subject). In some embodiments, the biological sample comprises a blood sample, a buccal sample, or a biopsy sample (e.g., a liquid biopsy sample). In some embodiments, a biopsy sample comprises frozen tissue or formalin-fixed paraffin-embedded (FFPE) tissue. In some embodiments, a biopsy sample (e.g., a liquid biopsy sample) comprises cell-free DNA or DNA from circulating tumor cells.

In some embodiments, tagged amplicons are generated by amplifying two or more ROIs using PCR (e.g., multiplex PCR). In some embodiments, tagged amplicons are generated by amplifying two or more ROIs using multiplex PCR. In some embodiments, the PCR and/or multiplex PCR comprises magnesium in a working concentration of about 1.5 mM to about 3 mM. In some embodiments, the PCR and/or multiplex PCR comprises DMSO in a working concentration of about 3% to about 6% by volume (v/v). In some embodiments, the PCR and/or multiplex PCR comprises a pH of about 8.5 to about 9.2. In some embodiments, amplifying two or more ROIs comprises amplifying at least two, at least 5, at least 10, at least 20, at least 30, at least 40, or at least 50 ROIs. In some embodiments, amplifying two or more ROIs comprises amplifying at least 2, 3, 4, 5, 6, 7, 8, 9, 10, or more, e.g., at least 12, or at least 14 ROIs. In some embodiments, each ROI is about 2, about 5, about 10, about 20, about 30, about 40, about 50, about 100, about 150, about 200, about 250, about 500, about 1,000, about 2,000, about 5,000, or about 10,000 nucleotides in length.

In some embodiments, tagged amplicons are generated by amplifying two or more ROIs using a set of tagged, sequence-specific primers in a PCR reaction (e.g., a multiplex PCR reaction, e.g., a multiplex PCR reaction in a single tube). In some embodiments, a 5′ tag sequence is an artificial tag sequence. In some embodiments, a 5′ tag sequence is an artificial tag sequence that is not homologous (e.g., is less than 70% identical) to a human genome sequence. In some embodiments, the tagged, sequence-specific primers are designed such that the 5′ tag sequence of the reverse primer for each ROI is complementary to the 5′ tag sequence of the forward primer for another ROI. In some embodiments, the tagged, sequence-specific primers are designed such that the 5′ tag sequence of the reverse primer for each ROI is complementary to the 5′ tag sequence of the forward primer for the ROI immediately downstream. In some embodiments, the tagged, sequence-specific primers are designed such that the 5′ tag sequence of the reverse primer for each ROI is complementary to the 5′ tag sequence of the forward primer for an ROI that is not immediately downstream. In some embodiments, the tagged, sequence-specific primers are designed as shown in FIG. 1 for the target nucleic acid (i.e., 5′ Tag, of reverse primer of Exon₁ is complementary to a 5′ rcTag₁ of forward primer of Exon₂, a 5′ Tag₂ of reverse primer of Exon₂ is complementary to a 5′ rcTag₂ of forward primer of Exon₃, etc.). In some embodiments, the order of the one or more concatenated amplicons is identical to the order of the corresponding ROIs in the target nucleic acid. In some embodiments, the ratio of the one or more concatenated amplicons to the corresponding ROIs in the target nucleic acid is about 1 to 1.

Following amplification, in some embodiments, the amplicons comprise complementary tag sequences, which allow the tagged amplicons to be assembled into a single concatenated product. In some embodiments, the total length of the one or more concatenated amplicons is about 2,000 to about 50,000 nucleotides (e.g., about 3,000, about 4,000, about 5,000, or about 10,000 nucleotides, or longer). In some embodiments, concatenating the tagged amplicons comprises providing a DNA polymerase. In some embodiments, the DNA polymerase has 3′ to 5′ exonuclease activity. In some embodiments, the DNA polymerase is a high-fidelity DNA polymerase. In some embodiments, the DNA polymerase is a high-fidelity DNA polymerase (e.g., a Q5, Pfu, or Kapa HiFi HotStart DNA polymerase) and the PCR and/or multiplex PCR conditions comprise magnesium, e.g., in a working concentration of about 1.5 mM to about 3 mM. In some embodiments, the DNA polymerase is a high-fidelity DNA polymerase (e.g., a Q5, Pfu, or Kapa HiFi HotStart DNA polymerase) and the PCR and/or multiplex PCR conditions comprise DMSO, e.g., in a working concentration of about 3% to about 6% by volume (v/v). In some embodiments, the DNA polymerase is a high-fidelity DNA polymerase (e.g., a Q5, Pfu, or Kapa HiFi HotStart DNA polymerase) and the PCR and/or multiplex PCR conditions comprise a pH of about 8.5 to about 9.2. In some embodiments, the DNA polymerase is a Q5, Pfu, or Kapa HiFi HotStart DNA polymerase. In some embodiments, concatenating the tagged amplicons comprises providing at least one adjuvant. In some embodiments, the at least one adjuvant comprises TMAC, ThermaGo, and/or ThermaStop.

In some embodiments, the working concentration of one or more primers in step (i) is about 30 nM. In some embodiments, one or more primers in step (i) are depleted prior to concatenating the tagged amplicons. In some embodiments, one or more primers are depleted via purification.

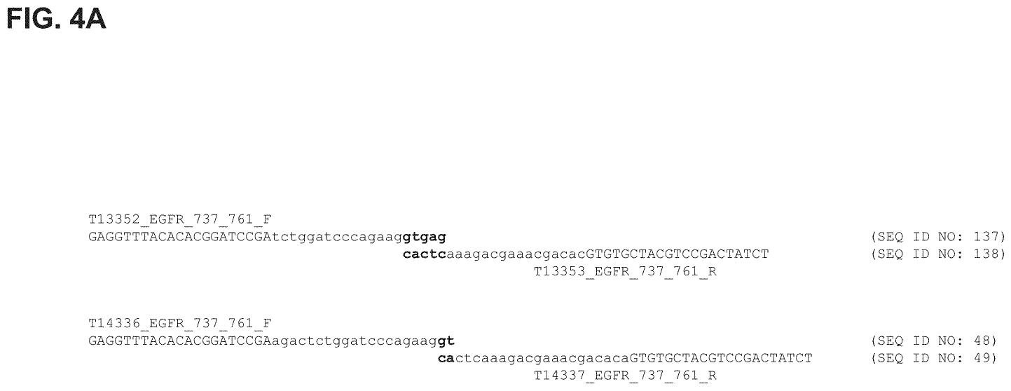

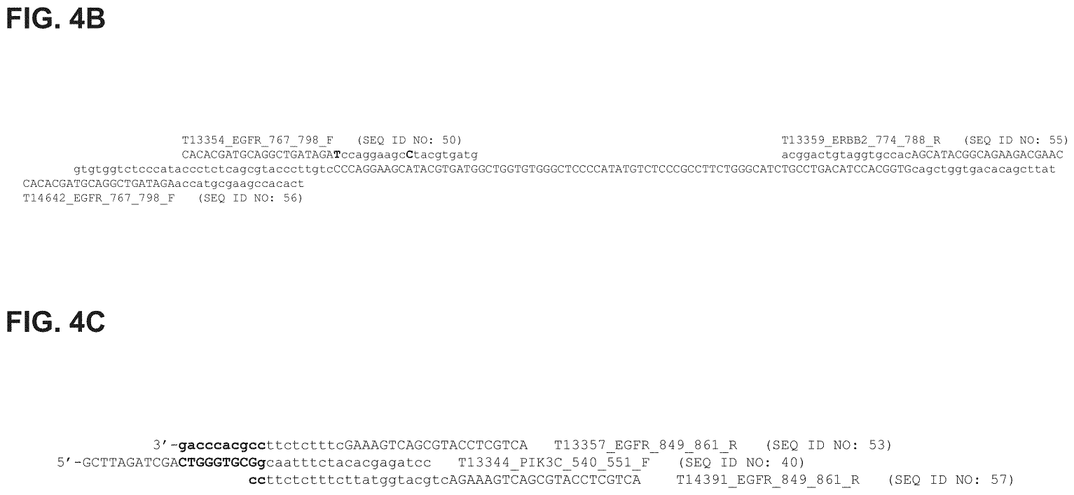

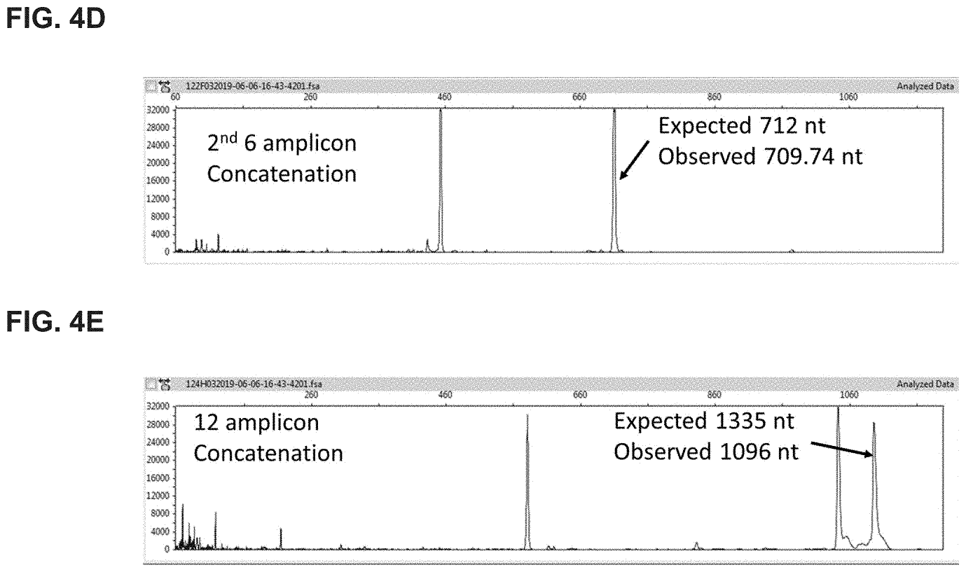

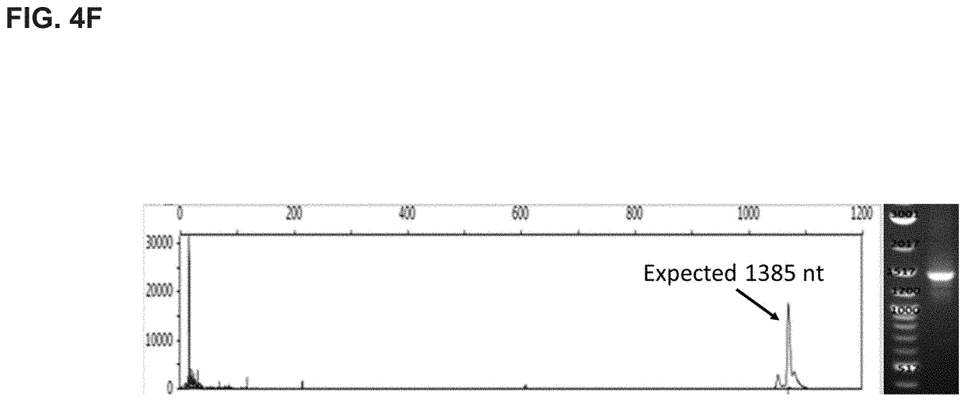

In some embodiments, one or more primers in step (i) are selected to prevent formation of one or more primer dimers. In some embodiments, selection comprises designing one or more primers in step (i) to comprise minimal sequence that is capable of hybridizing to an ROI and also complementary to a sequence in another primer. Exemplary primers comprising minimal sequence that is capable of hybridizing to an ROI and also complementary to a sequence in another primer are described and exemplified herein (e.g., in Example 2 and Table 4; see also FIG. 4A-4C, which show exemplary strategies for selecting and/or designing primers in order to eliminate, e.g., an exponentially-amplifiable primer dimer (FIG. 4A), an off-target amplification (FIG. 4B), or a linearly-amplifiable primer dimer (FIG. 4C). In some embodiments, the minimal sequence is at least about 6 nucleotides in length. In some embodiments, the minimal sequence is about 15 to about 30 nucleotides in length. In some embodiments, the minimal sequence is about 18 to about 20 nucleotides in length. In some embodiments, the minimal sequence comprises a sequence or a portion of a sequence set forth in Table 4 and the PCR and/or multiplex PCR conditions comprise magnesium, e.g., in a working concentration of about 1.5 mM to about 3 mM. In some embodiments, the minimal sequence comprises a sequence or a portion of a sequence set forth in Table 4 and the PCR and/or multiplex PCR conditions comprise DMSO, e.g., in a working concentration of about 3% to about 6% by volume (v/v). In some embodiments, the minimal sequence comprises a sequence or a portion of a sequence set forth in Table 4 and the PCR and/or multiplex PCR conditions comprise a pH of about 8.5 to about 9.2.

In some embodiments, one or more primers in step (i) are selected to minimize formation of one or more dead-end intermediate products, e.g., products that cannot form one or more concatenated amplicons. In some embodiments, selection comprises designing one or more primers in step (i) to comprise at least one adenine between the 5′ tag sequence and the sequence capable of hybridizing to the ROI.

In some embodiments, one or more primers in step (i) do not comprise a molecular barcode. In other embodiments, one or more primers in step (i) comprise a molecular barcode. In some embodiments, one or more primers comprise a barcode within the 5′ tag sequence. In some embodiments, a barcode included within the 5′ tag sequence labels each tagged amplicon with a unique barcode sequence. In some embodiments, one or more primers comprising a barcode are depleted after amplification, e.g., via purification, to remove any unincorporated molecular barcode primers from the reaction mixture (e.g., after PCR and/or multiplex PCR). In some embodiments, following sequencing in step (v), the number of unique barcodes in the final sequencing reads are counted and the copy number of input molecules is determined. In some embodiments, following amplification, concatenation, and sequencing, the number of unique barcode sequences incorporated into a concatemer are counted and compared to reference counts for a known copy-number gene. In some embodiments, the copy number of the target gene is calculated based on the molecular barcode counting ratio relative to the reference gene.

In some embodiments, end primers with tag sequences are used to drive amplification of a concatenated amplicon (e.g., TagA and TagB primers in FIG. 1, or the like). In some embodiments, a first end primer is capable of hybridizing to a tag sequence at the 5′ end of a concatenated amplicon. In some embodiments, a second end primer is capable of hybridizing to a tag sequence at the 3′ end of a concatenated amplicon. In some embodiments, the tag sequence at the 5′ end of the concatenated amplicon is identical to or overlaps with the 5′ tag sequence of a forward primer used to amplify an ROI in step (i). In some embodiments, the tag sequence at the 3′ end of the concatenated amplicon is identical to or overlaps with the 5′ tag sequence of a reverse primer used to amplify an ROI in step (i). In some embodiments, the first end primer and the second end primer are added in any one of steps (i)-(iii). In some embodiments, the first end primer and the second end primer are added in step (i) and the method comprises 1-step PCR. In other embodiments, the first end primer and the second end primer are added in step (ii) or step (iii) and the method comprises 2-step PCR

In some embodiments, sequencing in step (v) comprises single-molecule sequencing. In some embodiments, the sequencing comprises long-read sequencing (e.g., sequencing about 800 nucleotides or longer). In some embodiments, the sequencing comprises nanopore sequencing or single-molecule real-time (SMRT) sequencing. In some embodiments, the sequencing comprises long-read sequencing of a target nucleic acid, e.g., using the method described above or any of the exemplary methods described herein.

In some embodiments, a target nucleic acid comprises one or more genes or a multiple gene panel. In some embodiments, the one or more genes comprise a human gene. In some embodiments, the human gene is a human disease gene. In some embodiments, the human gene is a human cancer gene. In some embodiments, the one or more genes comprise CFTR, SMN1, SMN2, KRAS, BRAF, PIK3C. EGFR, and/or ERBB2. In some embodiments, the human gene is a human gene with high modeled fetal disease risk (MFDR). In some embodiments, the one or more genes comprise SMN1, SMN2, FMR1, HBA1, HBA2, and/or GBA. In some embodiments, the one or more genes comprise CFTR, FMR1, SMN1, SMN2, IKBKAP, ABCC8, FANCC, GALT, GBA, G6PC, HBA1, HBA2, HBB, BLM, ASPA, TMEM216, BCKDHA, BCKDHB, ACADM, MCOLN1, NEB, SMPD1, F8, HEXA, PCDH15, DMD, CYP21A2, and/or CLRN1. In some embodiments, the one or more genes comprise CFTR, FMR1, SMN1, and/or SMN2.

In some embodiments, a target nucleic acid is used in a multiple gene panel. In some embodiments, a target nucleic acid is used in a multiple gene panel, e.g., to detect mutations and/or structural variation in one or more target genes. In some embodiments, the multiple gene panel is a newborn or carrier screening panel. In some embodiments, the multiple gene panel comprises one or more human genes. In some embodiments, the human gene(s) is/are human disease gene(s). In some embodiments, the methods and nucleic acid libraries disclosed herein are used to detect the presence or absence of a mutation in one or more of the human disease genes, e.g., in the newborn or carrier screening panel. In some embodiments, the human gene is a human cancer gene. In some embodiments, the multiple gene panel comprises CFTR, SMN1, SMN2, KRAS, BRAF, PIK3C, EGFR, and/or ERBB2. In some embodiments, the multiple gene panel comprises SMN1, SMN2, FMR1, HBA1, HBA2, and/or GBA. In some embodiments, the multiple gene panel comprises CFTR, FMR1, SMN1, SMN2, IKBKAP, ABCC8, FANCC, GALT, GBA, G6PC, HBA1, HBA2, HBB, BLM, ASPA, TMEM216, BCKDHA, BCKDHB, ACADM, MCOLN1, NEB, SMPD1, F8, HEXA, PCDH15, DMD, CYP21A2, and/or CLRN1. In some embodiments, the multiple gene panel comprises CFTR, FMR1, SMN1, and/or SMN2. In some embodiments, the human gene is a human gene with high modeled fetal disease risk (MFDR).

In some embodiments, a target nucleic acid and/or a multiple gene panel is used to detect a variation having clinical significance. Without wishing to be bound by theory, the clinical significance of any given sequence variant typically falls along a gradient, ranging from those in which the variant is almost certainly pathogenic for a disorder to those that are almost certainly benign. Various standards and guidelines for the classification of sequence variants have been developed using criteria informed by expert opinion and empirical data, such as the guidelines from the American College of Medical Genetics and Genomics (ACMG) (see, e.g., Richards et al., (2015) Genet Med 17(5):405-24, which is incorporated herein by reference). As used herein, the term “modeled fetal disease risk” or “MDFR” refers to the probability that a hypothetical fetus created from a random pairing of individuals would be homozygous or compound heterozygous for two mutations presumed to cause severe or profound disease (i.e., a disease that if left untreated would cause intellectual disability, a substantially shortened lifespan, or both). A gene with “high” MDFR, as used herein, means a gene having one or more sequence variants classified as pathogenic or likely pathogenic (e.g., as determined, e.g., using ACMG guidelines) and presumed to cause “profound” disease (e.g., as determined, e.g., using the algorithm described in Lazarin et al., (2014) PLoS One. 2014; 9(12):e114391; see also Hague et al., (2016) JAMA 316(7):734-42, each of which is incorporated herein by reference).

In some embodiments, the multiple gene panel is a carrier screening panel. In some embodiments of the exemplary methods and compositions disclosed herein, nucleic acid variants relevant to carrier screening are amplified and/or captured in about 200 to about 400 discrete (short) amplicons (e.g., about 180 to about 220, about 220 to about 260, about 260 to about 300, about 300 to about 340, about 340 to about 380, or about 380 to about 420 discrete (short) amplicons). In some embodiments of the exemplary methods and compositions disclosed herein, sample input is less than about 2 μg of a template nucleic acid (e.g., template DNA), e.g., less than about 1.9 μg, less than about 1.8 μg, less than about 1.7 μg, less than about 1.6 μg, less than about 1.5 μg, less than about 1.4 μg, less than about 1.3 μg, less than about 1.2 μg, less than about 1.1 μg, or less than about 1.0 μg. In some embodiments, sample input is less than about 1 μg of a template nucleic acid (e.g., template DNA), e.g., less than about 0.9 μg, less than about 0.8 μg, less than about 0.7 μg, less than about 0.6 μg, or less than about 0.5 μg.

In some embodiments of the exemplary methods and compositions disclosed herein, the discrete (short) amplicons are concatenated into about 10 to about 50 concatenated amplicons (e.g., about 5 to about 20, about 15 to about 30, about 25 to about 40, about 35 to about 50, about 45 to about 60 concatenated amplicons). In some embodiments, the concatenated amplicons are sequenced using, e.g., single-molecule sequencing or any long-read sequencing platform. In some embodiments, the disclosed methods and compositions can be applied to sequencing across panels of different disease genes and/or markers.

In some embodiments, a target nucleic acid is from a sample (e.g., a biological sample). In some embodiments, a target nucleic acid is from a biological sample. In some embodiments, a target nucleic acid is isolated or purified from a biological sample, e.g., by a process which comprises removing one or more non-nucleic acid components from the biological sample.

As used herein, the term “sample” refers to any composition containing or presumed to contain a target nucleic acid. A sample isolated from a subject, i.e., separated from one or more of the conditions or factors present naturally in the subject, may be referred to as a “biological sample.” A biological sample can be obtained from a living subject, or can be obtained from a subject post-mortem. A biological sample can comprise cell culture constituents, such as, e.g., cultured cells, conditioned media, recombinant cells, and cell components. In some embodiments, a biological sample comprises cells. Cells can be primary cells, can be immortalized cells from a cell line, can be mammalian, or can be non-mammalian (e.g., bacteria, yeast). In some embodiments, a biological sample comprises cell components.

In some embodiments, a biological sample is obtained from a subject. The term “subject” refers to any biological entity comprising genetic material. For example, the subject can be an animal, plant, fungus, or microorganism, such as, e.g., a bacterium, virus, archaeon, microscopic fungus, or protist. In some embodiments, the subject is a human or non-human animal. Non-human animals include all vertebrates (e.g., mammals and non-mammals). In some embodiments, the subject is a mammal. In some embodiments, the subject is a human. In some embodiments, the subject is not diagnosed with and/or is not suspected of being at risk for a disease. In some embodiments, the subject is diagnosed with and/or is suspected of being at risk for a disease. In some embodiments, the disease is a cancer.

Exemplary biological samples include, without limitation, samples of tissue or liquid isolated from a subject. Non-limiting examples of tissues include, e.g., brain, bone, marrow, lung, heart, esophagus, stomach, duodenum, liver, prostate, nerve, meninges, kidneys, endometrium, cervix, breast, lymph node, muscle, hair, and skin, among others. A biological sample can also comprise liquid (e.g., a fluid). Exemplary liquid biological samples include, e.g., whole blood, plasma, serum, soluble cellular extract, extracellular fluid, cerebrospinal fluid, ascites, urine, sweat, tears, saliva, buccal sample, a cavity rinse, or an organ rinse. A biological sample may also include samples of in vitro cultures established from cells taken from a subject, including formalin-fixed paraffin-embedded (FFPE) tissue and nucleic acids isolated therefrom. A sample (e.g., a biological sample) may also include cell-free material, such as cell-free blood fraction that contains cell-free DNA (cfDNA) or DNA from circulating tumor cells (ctDNA). Exemplary methods for lysing cells include but are not limited to mechanical disruption, liquid homogenization, high frequency sound waves, freeze/thaw cycles, and manual grinding. Other exemplary methods for lysing cells or otherwise extracting nucleic acids from a sample are known and would be apparent to one of skill in the art.

In some embodiments, multiple nucleic acids, including all the nucleic acids in a sample, may be converted to library molecules using the methods and compositions described herein. In some embodiments, a sample is a biological sample derived or isolated from a human.

In some embodiments, a biological sample comprises a blood sample. In some embodiments, a biological sample comprises a buccal sample. In some embodiments, a biological sample comprises a fragment of a solid tissue or a solid tumor derived from a human patient, e.g., by biopsy. In some embodiments, the biological sample comprises a biopsy sample. In some embodiments, the biopsy sample comprises frozen tissue or FFPE tissue. In some embodiments, the biopsy sample comprises a liquid biopsy sample. In some embodiments, the liquid biopsy sample comprises cfDNA or ctDNA.

The term “sequencing,” as used herein, refers to any method of determining the sequence of nucleotides in a target nucleic acid. In some embodiments, a library of concatenated amplicons (e.g., a library described herein and/or generated using any of the exemplary methods described herein) can be sequenced. In some embodiments, a library of concatenated amplicons described herein and/or generated using any of the exemplary methods described herein is particularly advantageous in single-molecule sequencing, or in any sequencing platform capable of long-reads (i.e., reads about 800 nucleotides in length, or longer). In some embodiments, sequencing comprises single-molecule sequencing. In some embodiments, sequencing comprises long-read sequencing. In some embodiments, sequencing comprises sequencing about 800 nucleotides or longer.

Non-limiting examples of such long-read sequencing technologies include, without limitation, platforms using single-molecule real-time (SMRT) sequencing such as SMRT by Pacific Biosciences (Menlo Park, Calif., USA), and platforms using nanopore sequencing such as biological nanopore-based instruments manufactured by Oxford Nanopore Technologies (Oxford, UK) or Roche Genia (Santa Clara, Calif., USA) or solid state nanopore-based instruments described, e.g., in WO 2016/142925 and Stranges et al., (2016) PNAS 113(44):E6749, and any other presently existing or future single-molecule sequencing technology that is suitable for long-reads. Exemplary long-read sequencing methods and instruments are also described, e.g., in Liu et al., (2017) Genome Med. 9(1):65; Gieβelmann et al., (2018) “Repeat expansion and methylation state analysis with nanopore-sequencing,” (DOI: 10.1101/480285); Cheng et al., (2015) Clin Chem. 61(10):1305-6; Wei et al., (2018) Fertil Steril. 110(5):910-6; Leija-Salazar et al., (2019) Mol Genet Genomic Med, 7(3):e564; and U.S. Pat. Nos. 8,828,208, 9,057,102, 9,404,146, and 9,542,527, each of which is incorporated herein by reference for the disclosure of such methods and instruments. In some embodiments, sequencing comprises SMRT sequencing or nanopore sequencing.

In some embodiments, the compositions and methods disclosed herein can be used for structural variation characterization, e.g., of a nucleic acid in a sample. In some embodiments, structural variation characterization comprises detecting or quantifying single nucleotide variants (SNV), repeat sequences, indels, gene chimera, and/or gene copy number. In some embodiments, detecting or quantifying gene copy number comprises detecting or quantifying one or more molecular barcodes. In some embodiments, one or more molecular barcodes are used to quantify the original copy input of each ROI. In some embodiments, detecting or quantifying gene copy number comprises using and/or comparing to an external spiking control. In some embodiments, an external spiking control is used to quantify the original copy input of each ROI. In some embodiments, the external spiking control comprises a synthetic gBlock control. In some embodiments, the copy input information is used to detect copy number variation. In some embodiments, the one or more molecular barcodes are in one or more primers. In some embodiments, structural variation characterization comprises labeling and/or direct imaging.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1

### Amplicon Concatenation from QuantideX® NGS DNA Hotspot 21 Kit

To determine whether 46 short amplicons from a QuantideX® NGS DNA Hotspot 21 Kit for cancer mutation detection (Asuragen) can be converted into one longer amplicon, 12 amplicons from the 46-amplicon panel were selected (Table 1). The end primer tags included Illumina P5, AATGATACGGCGACCACCGA (SEQ ID NO: 1) for T14007_KRAS_4_15_F2 and lllumina P7, CAAGCAGAAGACGGCATACGA (SEQ ID NO: 2) for T14008_ERBB2_774_788_R2. All other complementary tag sequences were derived from natural (genomic) sequence. For instance, in the tag sequence AGGACTGGGGTTTTATTATA (SEQ ID NO: 3) for T13984_KRAS_4_15_R, the TTTTATTATA portion (SEQ ID NO: 4) was adjacent to the natural gene-specific portion of the KRAS_4_15 sequence, while the AGGACTGGGG portion was reverse complementary to the gene-specific sequence of the KRAS_55_65_F primer.

Three primer pools were made. Primer pool#1 had 12 primers at 500 nM each from the 1^{st} 6 amplicons (Table 1). Primer pool#2 had 12 primers at 500 nM each from the 2^{nd} 6 amplicons (Table 1). Primer pool#3 had the complete set of 24 primers at 500 nM each. A 10 μl PCR reaction contained 5 μl of 2× Phoenix Taq PCR master mix (Enzymatics), 1 μl of 10 ng/μl DNA (NA12878, Coriell), 1 μl of 500 mM TMAC, 1 μl of 500 nM primer pool (#1 or #2 or #3), and 2 μl of nuclease-free water. The pre-amplification cycle conditions were 95° C./5 min, 2 cycles of 95° C./15 sec, 64° C./4 min, 28 cycles of 95° C./15 sec, 72° C./4 min. The reactions were paused at 72° C. on the thermal cycler at the end of the first PCR and 1 μl of 15 μM tagging primer mix was added. For reactions using primer pool#1, primer pool#2, or primer pool#3, a tagging primer of T2109-FAM-P5/T13994, T13995/T2110-P7-FAM, and T2109-FAM-P5/T2110-P7 was used, respectively. After end primer was added, the reactions resumed with 25 cycles 95° C./15 sec, 55° C./1 min, 72° C./2 min, and a final 72° C./10 min 4° C. hold. The final PCR products were diluted 1:50 fold and 1 μl was mixed with 12 μl of HiDi (ABI) and 2 μl of ROX1000 size standard (Asuragen). Capillary electrophoresis (CE) was run at 2.5 KV for 20 sec inject and 20 KV for 40 min run.

The expected full length product sequences of the 1^{st} 6 and the 2^{nd} 6 amplicons are set forth in Table 2. The expected sequence of the assembled 12-amplicon concatenation product is set forth in Table 3.

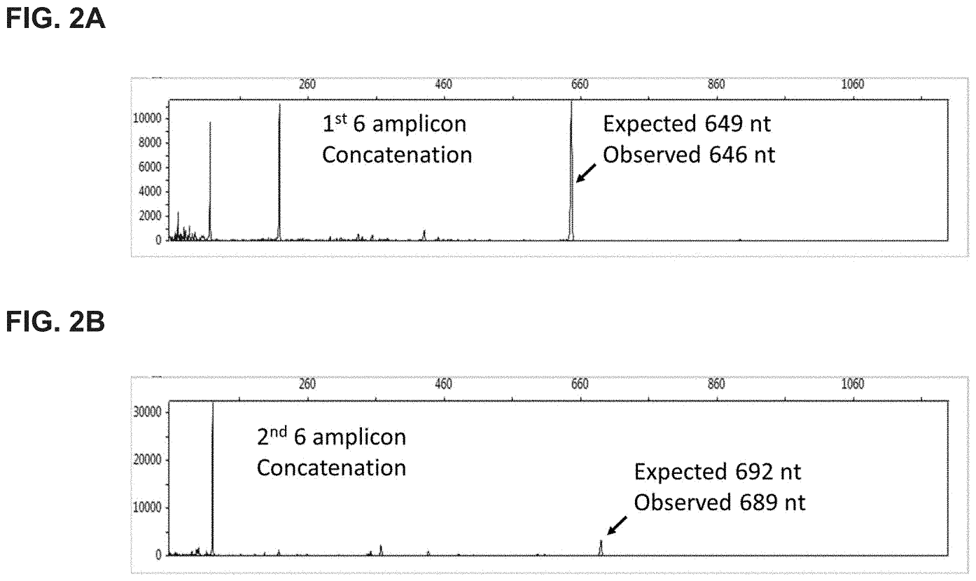

The full length product of the 1^{st} 6 amplicons was detected with an observed size of 646 nt (with primer pool#1) (FIG. 2A). The full length product of the 2^{nd} 6 amplicons was detected with an observed size of 689 nt (with primer pool#2) (FIG. 2B). The full length product of the assembled 12 amplicons was not detected (with primer pool#3). Without wishing to be bound by theory, formation of primer dimers and/or use of natural (non-artificial) tag sequences may have prevented detection of this full length product.

TABLE 1
Amplicon Version 1 (V1) Designs for Concentration.

| | Primer ID | SEQ ID NO | Primer Sequence* |
|---|---|---|---|
| 1^{st} 6 | T13983_KRAS_4_15_F | 5 | AATGATACGGCGACCACCGActgt |
| Amplicons | | | atcgtcaaggcactct |
| | T13984_KRAS_4_14_R | 6 | AGGACTGGGGTTTTATTATAaggc |
| | | | ctgctgaaaatgactg |
| | T13985_KRAS_55_65_F | 7 | TATAATAAAACCCCAGTCCTcatg |
| | | | tactggtccctcattg |
| | T13986_KRAS_55_65_R | 8 | GTAAGAATTGAGGCTAGTAATTGA |
| | | | tggagaaacctgtctcttgg |
| | T13987_BRAF_591_612_F | 9 | TCAATTACTAGCCTCAATTCTTAC |
| | | | catccacaaaatggatccagac |
| | T13988_BRAF_591_612_R | 10 | AATCTGCCCATCCTCAGATAtatt |
| | | | tcttcatgaagacctcacag |
| | T13989_BRAF_465_474_F | 11 | TATCTGAGGATGGGCAGATTacag |
| | | | tgggacaaagaattgga |
| | T14009_BRAF_465_474_R | 12 | TTTGAGCTGTACAATGTCACcaca |
| | | | ttacatacttaccatgccact |
| | T13991_PIK3C_540_551_F | 13 | GTGACATTGTACAGCTCAAAgcaa |
| | | | tttctacacgagatcc |
| | T13992_PIK3C_541_551_R | 14 | TTTATCTAAGGCATCTCCATTTta |
| | | | gcacttacctgtgactcc |
| | T13993_PIK3C_1038_1049_F | 15 | AAATGGAGATGCCTTAGATAAAac |
| | | | tgagcaagaggctttgg |
| | T13994_PIK3C_1038_1049_R | 16 | TTTTTCCAGTGAAGATCCAAtcca |
| | | | tttttgttgtccagcc |
| 2^{nd} 6 | T13995_EGFR_486_493_F | 17 | TTGGATCTTCATGGAAAAAactg |
| Amplicons | | | tttgggacctccggt |
| | T13996_EGFR_486_493_R | 18 | TTGGTTGGAAAGCGGTGacttact |
| | | | gcagctgttttcacctct |
| | T13997_EGFR_709_721_F | 19 | CACCGCTTTCCAACCAAgctctct |
| | | | tgaggatcttgaag |
| | T13998_EGFR_709_721_R | 20 | GTCCCTATGAGGGACCTTAcctta |
| | | | tacaccgtgccgaac |
| | T13999_EGFR_737_761_F | 21 | TAAGGTCCCTCATAGGGACtctgg |
| | | | atcccagaaggtgag |
| | T14010_EGFR_737_761_R | 22 | GGGAGGGAACCtCCAcacagcaaa |
| | | | gcagaaactcac |
| | T14001_EGFR_767_798_F | 23 | TGGAGGTTCCCTCCCtccaggaag |
| | | | cctacgtgatg |
| | T14002_EGFR_767_798_R | 24 | TCCTGGCTGATTGTCTTTGtgttc |
| | | | ccggacatagtccag |
| | T14003_EGFR_849_861_F | 25 | CAAAGACAATCAGCCAGGAacgta |
| | | | ctggtgaaaacaccg |
| | T14004_EGFR_849_861_R | 26 | AAGGGTACGCATGGTATTctttct |
| | | | cttccgcacccag |
| | T14005_ERBB2_774_788_F | 27 | AATACCATGCGTACCCTTgtcccc |
| | | | aggaagcatacgt |
| | T14006_ERBB2_774_788_R | 28 | CAAGCAGAAGACGGCATACGAcac |
| | | | cgtggatgtcaggca |

*Gene-specific portion of primer in lower case; tag portion of primer in upper
case.

TABLE 2
Concatenation Product Sequences.

| | SEQ ID NO | Expected Product Sequence |
|---|---|---|
| 1^{st} 6 | 29 | AATGATACGGCGACCACCGACTGTATCGTCAAGGCACTCTTGCCTACGC |
| Amplicons | | CACCAGCTCCAACTACCACAAGTTTATATTCAGTCATTTTCAGCAGGCC |
| (Expected | | TTATAATAAAACCCCAGTCCTCATGTACTGGTCCCTCATTGCACTGTAC |
| size: | | TCCTCTTGACCTGCTGTGTCGAGAATATCCAAGAGACAGGTTTCTCCAT |
| 649 nt) | | CAATTACTAGCCTCAATTCTTACCATCCACAAAATGGATCCAGACAACT |
| | | GTTCAAACTGATGGGACCCACTCCATCGAGATTTCACTGTAGCTAGACC |
| | | AAAATCACCTATTTTTACTGTGAGGTCTTCATGAAGAAATATATCTGAG |
| | | GATGGGCAGATTACAGTGGGACAAAGAATTGGATCTGGATCATTTGGAA |
| | | CAGTCTACAAGGGAAAGTGGCATGGTAAGTATGTAATGTGGTGACATTG |
| | | TACAGCTCAAAGCAATTTCTACACGAGATCCTCTCTCTGAAATCACTGA |
| | | GCAGGAGAAAGATTTTCTATGGAGTCACAGGTAAGTGCTAAAATGGAGA |
| | | TGCCTTAGATAAAACTGAGCAAGAGGCTTTGGAGTATTTCATGAAACAA |
| | | ATGAATGATGCACATCATGGTGGCTGGACAACAAAAATGGATTGGATCT |
| | | TCACTGGAAAAA |
| 2^{nd} 6 | 30 | TTGGATCTTCACTGGAAAAAACTGTTTGGGACCTCCGGTCAGAAAACCA |
| Amplicons | | AAATTATAAGCAACAGAGGTGAAAACAGCTGCAGTAAGTCACCGCTTTC |
| (Expected | | CAACCAAGCTCTCTTGAGGATCTTGAAGGAAACTGAATTCAAAAAGATC |
| size: | | AAAGTGCTGGGCTCCGGTGCGTTCGGCACGGTGTATAAGGTAAGGTCCC |
| 692 nt) | | TCATAGGGACTCTGGATCCCAGAAGGTGAGAAAGTTAAAATTCCCGTCG |
| | | CTATCAAGGAATTAAGAGAAGCAACATCTCCGAAAGCCAACAAGGAAAT |
| | | CCTCGATGTGAGTTTCTGCTTTGCTGTGTGGAGGTTCCCTCCCTCCAGG |
| | | AAGCCTACGTGATGGCCAGCGTGGACAACCCCCACGTGTGCCGCCTGCT |
| | | GGGCATCTGCCTCACCTCCACCGTGCAGCTCATCACGCAGCTCATGCCC |
| | | TTCGGCTGCCTCCTGGACTATGTCCGGGAACACAAAGACAATCAGCCAG |
| | | GAACGTACTGGTGAAAACACCGCAGCATGTCAAGATCACAGATTTTGGG |
| | | CTGGCCAAACTGCTGGGTGCGGAAGAGAAAGAATACCATGCGTACCCTT |
| | | GTCCCCAGGAAGCATACGTGATGGCTGGTGTGGGCTCCCCATATGTCTC |
| | | CCGCCTTCTGGGCATCTGCCTGACATCCACGGTGTCGTATGCCGTCTTC |
| | | TGCTTG |

To confirm whether the observed CE peaks of the 1^{st} and the 2^{nd} 6 amplicon concatenation reactions reflected the correct concatenation products, agarose gel was used to purify the two fragments of the 1^{st} 6 and the 2^{nd} 6 amplicon concatenation products. The fragments were then assembled in a separate PCR reaction with end primer T2109-FAM-P5/T2110-P7.

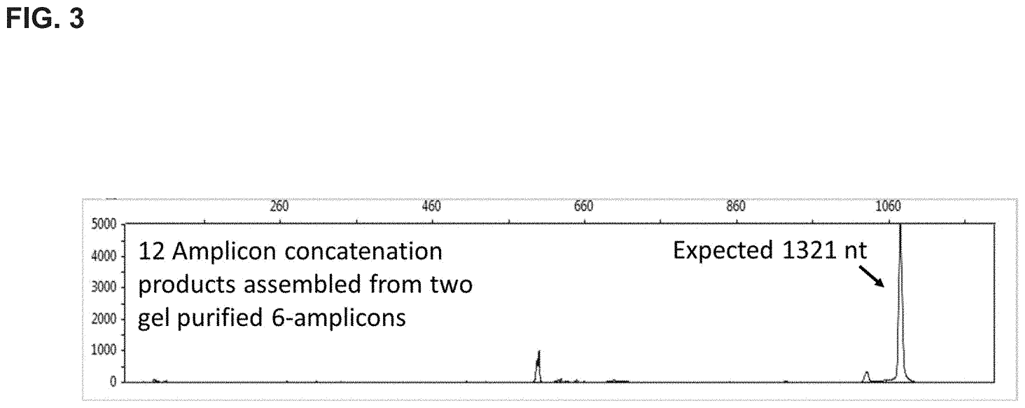

Single full length products were observed on CE (FIG. 3). The POP 7 polymer used on CE cannot resolve and size fragments greater than 1000 nt. The 1321 nt constructs therefore showed as about 1100 on CE. However, agarose gel analysis, nanopore sequencing, and Sanger sequencing all confirmed the full length of the 1321 nt constructs.

TABLE 3
Assembles Concatenation Product Sequence.

| | SEQ ID NO | Expected Product Sequence |
|---|---|---|
| 12 Amplicons | 31 | AATGATACGGCGACCACCGACTGTATCGTCAAGGCACTCTTGCC |
| (Expected size: | | TACGCCACCAGCTCCAACTACCACAAGTTTATATTCAGTCATTT |
| 1321 nt) | | TCAGCAGGCCTTATAATAAAACCCCAGTCCTCATGTACTGGTCC |
| | | CTCATTGCACTGTACTCCTCTTGACCTGCTGTGTCGAGAATATC |
| | | CAAGAGACAGGTTTCTCCATCAATTACTAGCCTCAATTCTTACC |
| | | ATCCACAAAATGGATCCAGACAACTGTTCAAACTGATGGGACCC |
| | | ACTCCATCGAGATTTCACTGTAGCTAGACCAAAATCACCTATTT |
| | | TTACTGTGAGGTCTTCATGAAGAAATATATCTGAGGATGGGCAG |
| | | ATTACAGTGGGACAAAGAATTGGATCTGGATCATTTGGAACAGT |
| | | CTACAAGGGAAAGTGGCATGGTAAGTATGTAATGTGGTGACATT |
| | | GTACAGCTCAAAGCAATTTCTACACGAGATCCTCTCTCTGAAAT |
| | | CACTGAGCAGGAGAAAGATTTTCTATGGAGTCACAGGTAAGTGC |
| | | TAAAATGGAGATGCCTTAGATAAAACTGAGCAAGAGGCTTTGGA |
| | | GTATTTCATGAAACAAATGAATGATGCACATCATGGTGGCTGGA |
| | | CAACAAAAATGGATTGGATCTTCACTGGAAAAAACTGTTTGGGA |
| | | CCTCCGGTCAGAAAACCAAAATTATAAGCAACAGAGGTGAAAAC |
| | | AGCTGCAGTAAGTCACCGCTTTCCAACCAAGCTCTCTTGAGGAT |
| | | CTTGAAGGAAACTGAATTCAAAAAGATCAAAGTGCTGGGCTCCG |
| | | GTGCGTTCGGCACGGTGTATAAGGTAAGGTCCCTCATAGGGACT |
| | | CTGGATCCCAGAAGGTGAGAAAGTTAAAATTCCCGTCGCTATCA |
| | | AGGAATTAAGAGAAGCAACATCTCCGAAAGCCAACAAGGAAATC |
| | | CTCGATGTGAGTTTCTGCTTTGCTGTGTGGAGGTTCCCTCCCTC |
| | | CAGGAAGCCTACGTGATGGCCAGCGTGGACAACCCCCACGTGTG |
| | | CCGCCTGCTGGGCATCTGCCTCACCTCCACCGTGCAGCTCATCA |
| | | CGCAGCTCATGCCCTTCGGCTGCCTCCTGGACTATGTCCGGGAA |
| | | CACAAAGACAATCAGCCAGGAACGTACTGGTGAAAACACCGCAG |
| | | CATGTCAAGATCACAGATTTTGGGCTGGCCAAACTGCTGGGTGC |
| | | GGAAGAGAAAGAATACCATGCGTACCCTTGTCCCCAGGAAGCAT |
| | | ACGTGATGGCTGGTGTGGGCTCCCCATATGTCTCCCGCCTTCTG |
| | | GGCATCTGCCTGACATCCACGGTGTCGTATGCCGTCTTCTGCTT |
| | | G |

### Example 2

### Amplicon Concatenation from QuantideX® NGS DNA Hotspot 21 Kit

To help detect the full length product of the assembled 12 amplicons from Example 1, agarose gel was used to purify the two 6-amplicon concatenation products. The two 6-amplicon concatenation products were then assembled using modified primers and modified PCR conditions to yield a 12-amplicon concatenation full length product in a single tube reaction without any purification in between.

Primers: Primers T13999_EGFR_737_761_F and T14010_EGFR_737_761_R have a perfectly matched stretch of 5 bases at their 3′ ends and are capable of forming a 78-bp primer dimer, which can result in an 80-bp deletion (FIG. 4A). Thus, to avoid truncated concatenation products, the sequences of these two primers were redesigned relative to the sequences used in Example 1 in order to prevent formation of primer dimers. All modified primers were also redesigned to comprise a bioinformatics-designed artificial tag sequence instead of a natural sequence (see Table 4).

TABLE 4
Amplicon Version 2 (V2) Designs for Concatenation.

| | Primer ID | SEQ ID NO | Primer Sequence* |
|---|---|---|---|
| 1^{st} 6 | T13336_KRAS_4_15_F | 32 | AATGATACGGCGACCACCGActct |
| Amplicons | | | atcgtcaaggcactct |
| | T13337_KRAS_4_15_R | 33 | CCTGGCTCCACAACCTAACGaggc |
| | | | ctgctgaaaatgactg |
| | T13338_KRAS_55_65_F | 34 | CGTTAGGTTGTGGAGCCAGGcatg |
| | | | tactggtccctcattg |
| | T13339_KRAS_55_65_R | 35 | CCTTGCACAGACCTGTCCAGtgga |
| | | | gaaacctgtctcttgg |
| | T13340_BRAF_591_612_F | 36 | CTGGACAGGTCTGTGCAAGGcatc |
| | | | cacaaaatggatccagac |
| | T13341_BRAF_591_612_R | 37 | GTGGGTAGGAACGTGCAGACtatt |
| | | | tcttcatgaagacctcacag |
| | T13342_BRAF_465_474_F | 38 | GTCTGCACGTTCCTACCCACacag |
| | | | tgggacaaagaattgga |
| | T13343_BRAF_465_474_R | 39 | CGCACCCAGTCGATCTAAGCcaca |
| | | | ttacatacttaccatgccact |
| | T13344_PIK3C_540_551_F | 40 | GCTTAGATCGACTGGGTGCGgcaa |
| | | | tttctacacgagatcc |
| | T13345_PIK3C_540_551_R | 41 | CAGCTGAAGAAGGCACGGTAtagc |
| | | | acttacctgtgactcc |
| | T13346_PIK3C_1038_1049_F | 42 | TACCGTGCCTTCTTCAGCTGactg |
| | | | agcaagaggctttgg |
| | T13347_PIK3C_1038_1049_R | 43 | CGCATAACTCGTTTCGCCTGtcca |
| | | | tttttgttgtccagcc |
| 2^{nd 6} | T13348_EGFR_486_493_F | 44 | CAGGCGAAACGAGTTATGCGactg |
| Amplicons | | | tttgggacctccggt |
| | T13349_EGFR_486_493_R | 45 | GGCCCATCCTCTGTTGCAATactt |
| | | | actgcagctgttttcacctct |
| | T13350_EGFR_709_721_F | 46 | ATTGCAACAGAGGATGGGCCgctc |
| | | | tcttgaggatcttgaag |
| | T13351_EGFR_709_721_R | 47 | TCGGATCCGTGTGTAAACCTCcct |
| | | | tatacaccgtgccgaac |
| | T14336_EGFR_737_761_F | 48 | GAGGTTTACACACGGATCCGAaga |
| | | | ctctggatcccagaaggt |
| | T14337_EGFR_737_761_R | 49 | TCTATCAGCCTGCATCGTGTGaca |
| | | | cagcaaagcagaaactcac |
| | T13354_EGFR_767_798_F | 50 | CACACGATGCAGGCTGATAGAtcc |
| | | | aggaagcctacgtgatg |
| | T13355_EGFR_767_798_R | 51 | CGACCTGGAAAGCCATTGTGAtgt |
| | | | tcccggacatagtccag |
| | T13356_EGFR_849_861_F | 52 | TCACAATGGCTTTCCAGGTCGacg |
| | | | tactggtgaaaacaccg |
| | T13357_EGFR_849_861_R | 53 | ACTGCTCCATGCGACTGAAAGctt |
| | | | tctcttccgcacccag |
| | T13358_ERBB2_774_788_F | 54 | CTTTCAGTCGCATGGAGCAGTgtc |
| | | | cccaggaagcatacgt |
| | T13359_ERBB2_774_788_R | 55 | CAAGCAGAAGACGGCATACGAcac |
| | | | cgtggatgtcaggca |

*Gene-specific portion of primer in lower case; tag portion of primer in upper
case.

Reaction Conditions: PCR cycling conditions were also modified relative to the conditions used in Example 1. The primers were mixed at 500 nM each and 0.6 μl were used in a 10 μl PCR reaction. The final primer concentration was 30 nM. The reaction contained 5 μl of 2× PhoenixTaq PCR master mix (Enzymatics), 1 μl of 10 ng/μl DNA (NA12878, Condi), 1 μl of 500 mM TMAC, 0.6 μl of 500 nM primer pool#2 (2^{nd} 6 amplicon pool) or pool#3 (complete set of 12 amplicon pool), and 2.4 μl of nuclease-free water. The pre-amplification and concatenation PCR conditions were 94° C./5 min, 2 cycles of 94° C./15 sec, 60° C./4 min, and 23 cycles of 94° C./15 sec, 72° C./2 min, followed by 20 cycles of 94° C./15 sec, 55° C./1 min, and 72° C./2 min (total PCR: 2 hours, 40 min), 1 μl of pre-amplification and concatenation PCR products were transformed into assembly/tagging PCR with 5 μl of 2× Phoenix Taq master mix, 1 μl of 15 μM T13348_EGFR_486_493_F and T2110-P7-FAM (for 2^{nd} 6 amplicon concatenation) or 1 μl of 15 μM T2109-P5-FAM and T2110-P7 (for 12 amplicon concatenation), and 3 μl of nuclease-free water. PCR cycle conditions were 95° C./5 min, 25 cycles of 95° C./15 sec, 55° C./1 min, and 72° C./2 min. The final PCR products were diluted 1:50 fold and 1 μl was used for CE.

With modified primer pools and PCR conditions, improved detection of the 2^{nd} 6 amplicon concatenation were observed (FIG. 4D). The full length 12-amplicon concatenation peak also showed as 1095 nt on CE (FIG. 4E).

In addition, primers T13354_EGFR_767_798_F and T13350_ERBB2_774_788_R were found to directly amplify the ERBB2 gene, resulting in a 260-bp truncation of PCR products (FIG. 4B). T13357_EGFR_849_861_R also paired with the concatenation tag sequence in T13344_PIK3C_540_551_F, resulting in a 748-bp deletion (FIG. 4C). After the primers were redesigned to avoid these nonspecific deletions (Table 5), full length products of the 12 amplicon concatenation were observed on CE and agarose gel (FIG. 4F).

TABLE 5
Redesign of Selected Primers in V2 Panel

| | |
|---|---|
| T14642_EGFR_ | CACACGATGCAGGCTGATAGAaccatgcgaagccac |
| 767_798_F | act |
| | (SEQ ID NO: 56) |
| T14391_EGFR_ | ACTGCTCCATGCGACTGAAAGActgcatggtattct |
| 849_861_R | ttctcttcc |
| | (SEQ ID NO: 57) |

### Example 3

### CFTR Amplicon Concatenation

To test the amplicon concatenation method on additional gene targets, 4 amplicons of the CFTR gene were designed to cover 24 common CFTR variants (Table 6). The expected sequence of the assembled 4-amplicon concatenation product is set forth in Table 7.

TABLE 6
CFTR Amplicon Designs for Concatenation.

| | SEQ | |
|---|---|---|
| Primer ID | ID NO | Primer Sequence* |
| T14028_G7-F | 58 | AATGATACGGCGACCACCGActgagacctta |
| | | caccgtttctca |
| T14036_G7-R | 59 | TGCGATGTGCCTGCTATGCTTGtcgcctctc |
| | | cctgctcaga |
| T14037_G8-F | 60 | CAAGCATAGCAGGCACATCGCAtgtcaaaga |
| | | tctcacagcaaaataca |
| T14038_G8-R | 61 | GGCCCATCCTCTGTTGCAATggcttctttag |
| | | ttattaacctagc |
| T14039_G9-F | 62 | ATTGCAACAGAGGATGGGCCatggggcctgt |
| | | gcaagga |
| T14040_G9-R | 63 | TCGGATCCGTGTGTAAACCTCtctctgtttt |
| | | tccccttttgt |
| T14041_G11_F | 64 | GAGGTTTACACACGGATCCGAtcttttgcag |
| | | agaatgggataga |
| T14035_G11-R | 65 | CAAGCAGAAGACGGCATACGAacctattcac |
| | | cagatttcgtagtc |
| | 66 | FAM-AATGATACGGCGACCACCGA |
| | 67 | CAAGCAGAAGACGGCATACGA |

*Gene-specific portion of primer in lower case; artificial
tag portion of primer in upper case.

TABLE 7
Assembled Concatenation Product Sequence.

| | SEQ ID NO | Expected Product Sequence |
|---|---|---|
| 4 Amplicons | 68 | AATGATACGGCGACCACCGACTGAGACCTTACACCGTTTCTCATTAGAA |
| (Expected size: | | GGAGATGCTCCTGTCTCCTGGACAGAAACAAAAAAACAATCTTTTAAAC |
| 1186 nt) | | AGACTGGAGAGTTTGGGGAAAAAAGGAAGAATTCTATTCTCAATCCAAT |
| | | CAACTCTATACGAAAATTTTCCATTGTGCAAAAGACTCCCTTACAAATG |
| | | AATGGCATCGAAGAGGATTCTGATGAGCCTTTAGAGAGAAGGCTGTCCT |
| | | TAGTACCAGATTCTGAGCAGGGAGAGGCGACAAGCATAGCAGGCACATC |
| | | GCAAGTCAAAGATCTCACAGCAAAATACACAGAAGGTGGAAATGCCATA |
| | | TTAGAGAACATTTCCTTCTCAATAAGTCCTGGCCAGAGGGTGAGATTTG |
| | | AACACTGCTTGCTTTGTTAGACTGTGTTCAGTAAGTGAATCCCAGTAGC |
| | | CTGAAGCAATGTGTTAGCAGAATCTATTTGTAACATTATTATTGTACAG |
| | | TAGAATCAATATTAAACACACATGTTTTATTATATGGAGTCATTATTTT |
| | | TAATATGAAATTTAATTTGCAGAGTCCTGAACCTATATAATGGGTTTAT |
| | | TTTAAATGTGATTGTACTTGCAGAATATCTAATTAATTGCTAGGTTAAT |
| | | AACTAAAGAAGCCATTGCAACAGAGGATGGGCCATGGGGCCTGTGCAAG |
| | | GAAGTATTACCTTCTTATAAATCAAACTAAACATAGCTATTCTCATCTG |
| | | CATTCCAATGTGATGAAGGCCAAAAATGGCTGGGTGTAGGAGCAGTGTC |
| | | CTCACAATAAAGAGAAGGCATAAGCCTATGCCTAGATAAATCGCGATAG |
| | | AGCGTTCCTCCTTGTTATCCGGGTCATAGGAAGCTATGATTCTTCCCAG |
| | | TAAGAGAGGCTGTACTGCTTTGGTGACTTCCTACAAAAGGGGAAAAACA |
| | | GAGAGAGGTTTACACACGGATCCGATCTTTTGCAGAGAATGGGATAGAG |
| | | AGCTGGCTTCAAAGAAAAATCCTAAACTCATTAATGCCCTTCGGCGATG |
| | | TTTTTTCTGGAGATTTATGTTCTATGGAATCTTTTTATATTTAGGGGTA |
| | | AGGATCTCATTTGTACATTCATTATGTATCACATAACTATATTCATTTT |
| | | TGTGATTATGAAAAGACTACGAAATCTGGTGAATAGGTTCGTATGCCGT |
| | | CTTCTGCTTG |

Reaction Conditions: The primers were mixed at 500 nM each and 0.6 μl were used in a 10 μl PCR reaction. The final primer concentration was 30 nM. The reaction contained 5 μl of 2× PhoenixTag PCR master mix (Enzymatics), 1 μl of 10 ng/μl DNA (NA12878, Coriell), 1 μl of 500 mM TMAC, 0.6 μl of 500 nM primer pool, and 2.4 μl of nuclease-free water. The pre-amplification and concatenation PCR conditions were 94° C./5 min, 2 cycles of 94° C./15 sec, 60° C./4 min, 23 cycles of 94° C./15 sec, 72° C./2 min, followed by 20 cycles of 94° C./15 sec, 55° C./1 min, and 72° C./2 min (total PCR: 2 hours, 40 min). 1 μl of pre-amplification and concatenation PCR products were transformed into assembly/tagging PCR with 5 μl of 2× Phoenix Taq master mix, 1 μl of 15 μM T2109-P5-FAM and T2110-P7, and 3 μl of nuclease-free water. PCR cycle conditions were 95° C./5 min, 25 cycles of 95° C./15 sec, 55° C./1 min, and 72° C./2 min. The final PCR products were diluted 1:50 fold and 1 μl was used for CE.

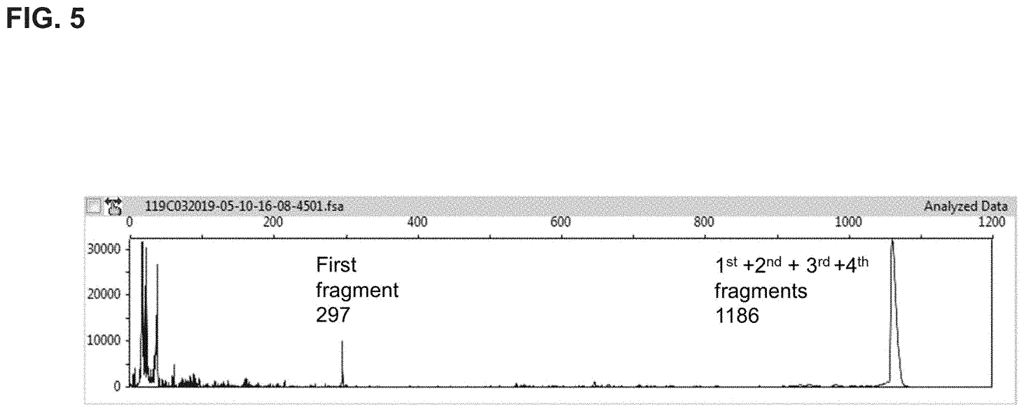

An exemplary CE trace of the concatenated products is shown in FIG. 5. The full length construct was observed on CE trace. For nanopore sequencing, the assembly/tagging PCR was performed without FAM-labeled primer. The PCR products were run on an agarose gel and purified with a PCR gel extraction kit (Zymo Research). The purified DNA concatenation products were sequenced by Nanopore MiniON flow cell (Oxford Nanopore Technologies).

Nanopore sequencing confirmed the correct 4-amplicon concatenation sequence (1186 nt). The full length 4-amplicon concatenation peak showed as 1059 nt on CE (FIG. 5).

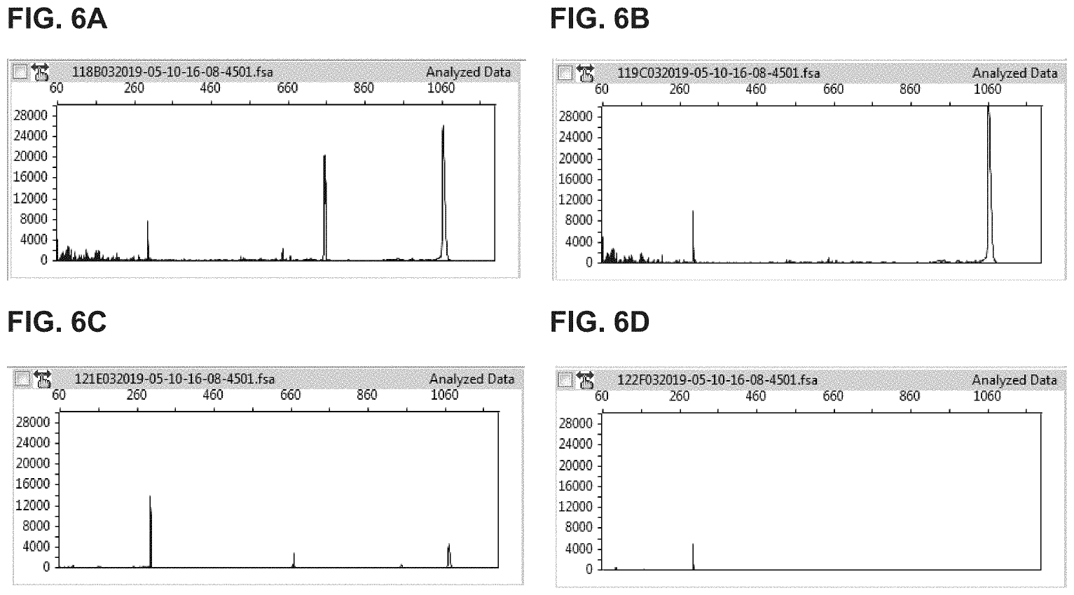

Primer concentrations were also varied by testing final primer concentrations of 5 nM, 10 nM, 30 nM, and 40 nM. The 30 nM final primer concentration produced the highest full length amplicon yield and least amount of truncated product (FIG. 6A-6D).

### Example 4

### Amplicon Concatenation Accommodating Extra “A” Overhang During PCR

Generally, when using a DNA polymerase which lacks 3′ to 5′ proofreading activity, the polymerase may acid a single, 3′ adenine (A) overhang to each end of the PCR product. Such non-template-based addition can have potential consequences for concatenation, e.g., preventing amplicons from further concatenation. For instance, in FIG. 5, the 297 nt peak is the first of four amplicons and some could not be fully incorporated into the full length concatenation product. The probability of this extra A addition is typically about 30-60%, but may be maximized if the PCR primers have one or more guanines (G) at the 5′ end. In contrast, DNA polymerases having 3′ to 5′ proofreading activity (e.g., high fidelity DNA polymerases such as Q5, Pfu, Kapa HiFi, etc.) are less likely to acid 3′ adenine overhangs. An alternative method for reducing the addition of 3′ adenine overhangs was also evaluated.

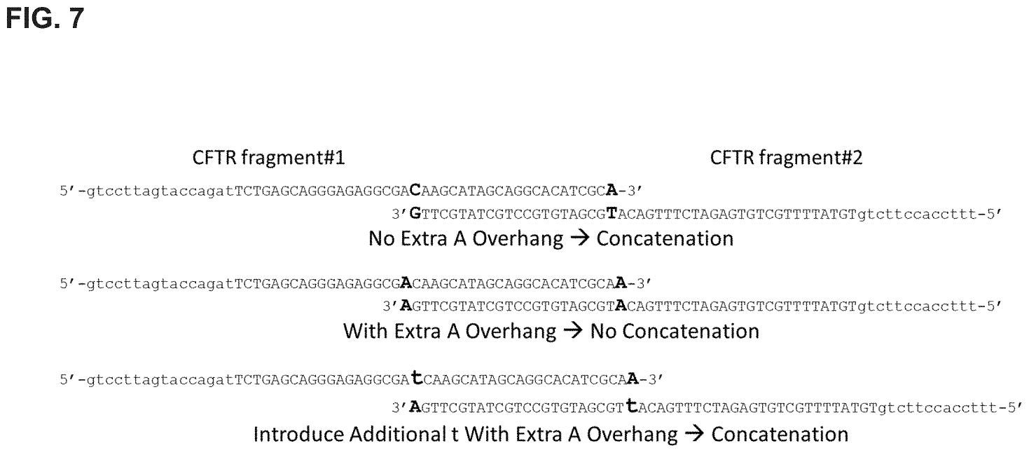

To investigate whether inserting an extra thymine (T) in a DNA template (e.g., as shown in FIG. 7) can accommodate a potential 3′ adenine overhang, modified primers having an extra adenine (A) were designed (Table 8) and used in a CFTR amplicon concatenation amplification. (Note: If the extra A is added in the forward primer, then the extra A will be represented in the final concatenation product. If the extra A is added in the reverse primer, then an extra T will be represented in the final concatenation product.) The expected sequence of the assembled 4-amplicon concatenation product with the extra A or T nucleotides is set forth in Table 9.

TABLE 8
Modified CFTR Amplicon Designs
for Concatenation.

| | SEQ | |
|---|---|---|
| Primer ID | ID NO | Primer Sequence* |
| T14028_G7-F | 69 | AATGATACGGCGACCACCGAactgagac |
| | | cttacaccgtttctca |
| T14076_GT-R | 70 | TGCGATGTGCCTGCTATGCTTGAtcgcc |
| | | tctccctgctcaga |
| T14077_G8-F | 71 | CAAGCATAGCAGGCACATCGCATTtgtc |
| | | aaagatctcacagcaaaataca |
| T14078_G8-R | 72 | GGCCCATCCTCTGTTGCAATAggcttct |
| | | ttagttattaacctagc |
| T14039_G9-F | 73 | ATTGCAACAGAGGATGGGCCatggggcc |
| | | tgtgcaagga |
| T14079_G9-R | 74 | TCGGATCCGTGTGTAAACCTCAtctctg |
| | | tttttccccttttgt |
| T14080_G11-F | 75 | GAGGTTTACACACGGATCCGAAtctttt |
| | | gcagagaatgggataga |
| T14035_G11-R | 76 | CAAGCAGAAGACGGCATACGAacctatt |
| | | caccagatttcgtagtc |
| T14028_G7-F | 77 | AATGATACGGCGACCACCGActgagacc |
| | | ttacaccgtttctca |
| T14076_G7-R | 78 | TGCGATGTGCCTGCTATGCTTGAtcgcc |
| | | tctccctgctcaga |

*Gene-specific portion of primer in lower case; artificial
tag portion of primer in upper case.

TABLE 9
Assembled Concatenation Product Sequence.

| | SEQ ID NO | Expected Product Sequence |
|---|---|---|
| 4 Amplicons | 79 | AATGATACGGCGACCACCGACTGAGACCTTACACCGTTTCTCATTAGAA |
| (Expected | | GGAGATGCTCCTGTCTCCTGGACAGAAACAAAAAAACAATCTTTTAAAC |
| size: | | AGACTGGAGAGTTTGGGGAAAAAAGGAAGAATTCTATTCTCAATCCAAT |
| 1191 nt) | | CAACTCTATACGAAAATTTTCCATTGTGCAAAAGACTCCCTTACAAATG |
| | | AATGGCATCGAAGAGGATTCTGATGAGCCTTTAGAGAGAAGGCTGTCCT |
| | | TAGTACCAGATTCTGAGCAGGGAGAGGCGATCAAGCATAGCAGGCACAT |
| | | CGCAATGTCAAAGATCTCACAGCAAAATACACAGAAGGTGGAAATGCCA |
| | | TATTAGAGAACATTTCCTTCTCAATAAGTCCTGGCCAGAGGGTGAGATT |
| | | TGAACACTGCTTGCTTTGTTAGACTGTGTTCAGTAAGTGAATCCCAGTA |
| | | GCCTGAAGCAATGTGTTAGCAGAATCTATTTGTAACATTATTATTGTAC |
| | | AGTAGAATCAATATTAAACACACATGTTTTATTATATGGAGTCATTATT |
| | | TTTAATATGAAATTTAATTTGCAGAGTCCTGAACCTATATAATGGGTTT |
| | | ATTTTAAATGTGATTGTACTTGCAGAATATCTAATTAATTGCTAGGTTA |
| | | ATAACTAAAGAAGCCTATTGCAACAGAGGATGGGCCATGGGGCCTGTGC |
| | | AAGGAAGTATTACCTTCTTATAAATCAAACTAAACATAGCTATTCTCAT |
| | | CTGCATTCCAATGTGATGAAGGCCAAAAATGGCTGGGTGTAGGAGCAGT |
| | | GTCCTCACAATAAAGAGAAGGCATAAGCCTATGCCTAGATAAATCGCGA |
| | | TAGAGCGTTCCTCCTTGTTATCCGGGTCATAGGAAGCTATGATTCTTCC |
| | | CAGTAAGAGAGGCTGTACTGCTTTGGTGACTTCCTACAAAAGGGGAAAA |
| | | ACAGAGATGAGGTTTACACACGGATCCGAATCTTTTGCAGAGAATGGGA |
| | | TAGAGAGCTGGCTTCAAAGAAAAATCCTAAACTCATTAATGCCCTTCGG |
| | | CGATGTTTTTTCTGGAGATTTATGTTCTATGGAATCTTTTTATATTTAG |
| | | GGGTAAGGATCTCATTTGTACATTCATTATGTATCACATAACTATATTC |
| | | ATTTTTGTGATTATGAAAAGACTACGAAATCTGGTGAATAGGTTCGTAT |
| | | GCCGTCTTCTGCTTG |

Reaction Conditions: The modified primers were mixed at 500 nM each and 0.6 μl were used in a 10 μl PCR reaction. The final primer concentration was 30 nM. The reaction contained 5 μl of 2× PhoenixTaq PCR master mix (Enzymatics), 1 μl of 10 ng/μl DNA (NA12878, Coriell), 1 μl of 500 mM TMAC, 0.6 μl of 500 nM modified primer pool, and 2.4 μl of nuclease-free water. The pre-amplification and concatenation PCR conditions were 94° C./5 min, 2 cycles of 94° C./15 sec, 60° C./4 min, 23 cycles of 94° C./15 sec, 72° C./2 min, followed by 20 cycles of 94° C./15 sec, 55° C./1 min, and 72° C./2 min (total PCR: 2 hours, 40 min). 1 μl of pre-amplification and concatenation PCR products were transformed into assembly/tagging PCR with 5 μl of 2× Phoenix Taq master mix, 1 μl of 15 μM T2109-P5-FAM and T2110-P7, and 3 μl of nuclease-free water. PCR cycle conditions were 95° C./5 min, 25 cycles of 95° C./15 sec, 55° C./1 min, and 72° C./2 min. The final PCR products were diluted 1:50 fold and 1 μl was used for CE.

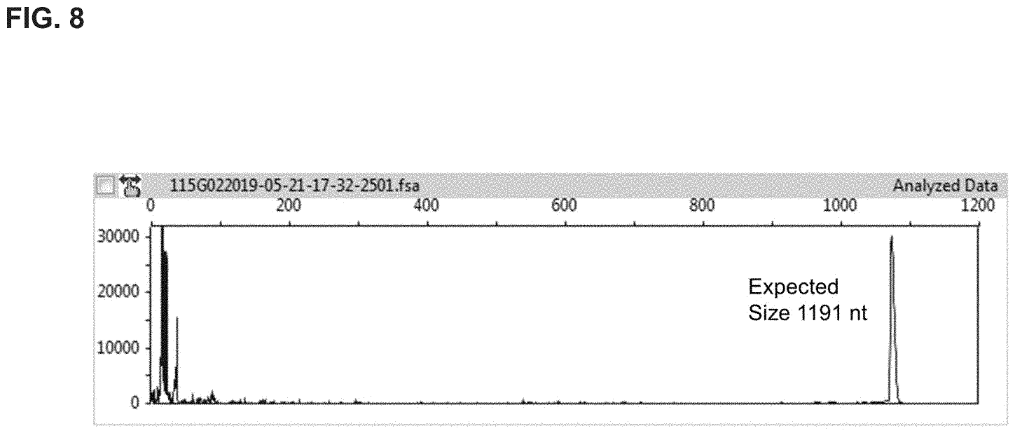

An exemplary CE trace of the concatenated products is shown in FIG. 8. The 297 nt peak was not detected (compare FIG. 8 to FIG. 5).

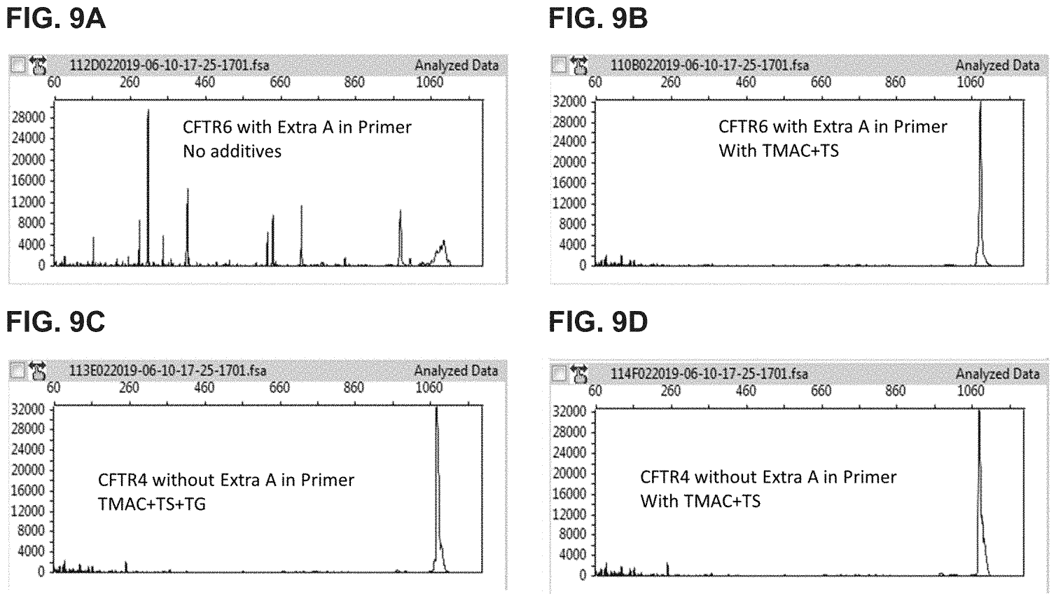

DNA polymerases were also varied by testing standard antibody-based HotStart Taq DNA polymerase and comparing to Kapa HiFi HotStart DNA polymerase. With or without an extra adenine in the primer design, Kapa HiFi HotStart DNA polymerase did not generate dead-end intermediate fragments (i.e., fragments which cannot be further concatenated into full length products), in contrast to standard antibody-based HotStart Taq DNA polymerase. However, the Kapa HiFi HotStart enzyme can have leak activity at lower temperatures, and may benefit from the addition of reagents such as TMAC, ThermaGo, and ThermaStop to suppress non-specific amplification (FIG. 9A-9D).

### Example 5

### CFTR Amplicon Concatenation

To test the amplicon concatenation method on additional CFTR variants (e.g., high frequency mutation variants), the DelF508 region and the G542X region were designed (Table 10) and added to the 4 amplicons of the CFTR gene. Exemplary variants covered by the 6 amplicons are listed in Table 11. The expected sequence of the assembled 6 amplicon concatenation product is set forth in Table 12.

TABLE 10
CFTR Amplicon Designs for Concatenation.

| | SEQ | |
|---|---|---|
| Primer ID | ID NO | Primer Sequence* |
| T14028_G7-F | 80 | AATGATACGGCGACCACCGActgaga |
| | | ccttacaccgtttctca |
| T14076_G7-R | 81 | TGCGATGTGCCTGCTATGCTTGAtcg |
| | | cctctccctgctcaga |
| T14077_G8_F | 82 | CAAGCATAGCAGGCACATCGCAAtgt |
| | | caaagatctcacagcaaaataca |
| G14078_G8-R | 83 | GGCCCATCCTCTGTTGCAATAggctt |
| | | ctttagttattaacctagc |
| T14039_G9-F | 84 | ATTGCAACAGAGGATGGGCCatgggg |
| | | cctgtgcaagga |
| T14079_G9-R | 85 | TCGGATCCGTGTGTAAACCTCAtctc |
| | | tgtttttccccttttgt |
| T14080_G11-F | 86 | GAGGTTTACACACGGATCCGAAtctt |
| | | ttgcagagaatgggataga |
| T14296_G11-R | 87 | TCTATCAGCCTGCATCGTGTGaccta |
| | | ttcaccagatttcgtagtc |
| T14297_Group10-F | 88 | CACACGATGCAGGCTGATAGAAtctt |
| | | acctcttctagttggcatgct |
| T14298_Group10-R | 89 | CGACCTGGAAAGCCATTGTGAAtggg |
| | | agaactggagccttca |
| T14299_Group01-F | 90 | TCACAATGGCTTTCCAGGTCGAgagc |
| | | atactaaaagtgactctctaattttc |
| T14300_Group01-R | 91 | CAAGCAGAAGACGGCATACGAcagca |
| | | aatgcttgctagacca |

*Gene-specific portion of primer in lower case; artificial
tag portion of primer in upper case.

TABLE 11
Exemplary Variants Covered by CFTR Amplicons.

| | | | | |
|---|---|---|---|---|
| 2347delG | R1162X | 405 + 3A > C | V520F-mut-F | 1717 − |
| | | | | 1G > A |
| 2307insA | R1158X | 394delTT | 1677delTA | G542X |
| 2184delA | 406 − 1G > A | G85E | I507del-mut-F | S549N |
| 2183AA > G | 444delA | R75X | F508del-mut-F | S549R |
| 2184insA | R117C | P67L | I506V-mut-F | G551D |
| 2143delT | R117H | E60X | F508C-mut-F | R553X |
| 3791delC | Y122X | G85E | I507V-mut-F | A559T |
| S1196X | I148T | | Q493X-mut-F | R560T- |
| | | | | mut-R |
| 3659delC | 621 + 1G > T | | G480C-mut-F | |

TABLE 12
Assembled Concatenation Product Sequence.

| | SEQ ID NO | Expected Product Sequence |
|---|---|---|
| 6 Amplicons | 92 | AATGATACGGCGACCACCGACTGAGACCTTACACCGTTTCTCATTAGAA |
| (Expected | | GGAGATGCTCCTGTCTCCTGGACAGAAACAAAAAAACAATCTTTTAAAC |
| size: | | AGACTGGAGAGTTTGGGGAAAAAAGGAAGAATTCTATTCTCAATCCAAT |
| 1589 nt) | | CAACTCTATACGAAAATTTTCCATTGTGCAAAAGACTCCCTTACAAATG |
| | | AATGGCATCGAAGAGGATTCTGATGAGCCTTTAGAGAGAAGGCTGTCCT |
| | | TAGTACCAGATTCTGAGCAGGGAGAGGCGATCAAGCATAGCAGGCACAT |
| | | CGCAATGTCAAAGATCTCACAGCAAAATACACAGAAGGTGGAAATGCCA |
| | | TATTAGAGAACATTTCCATCTCAATAAGTCCTGGCCAGAGGGTGAGATT |
| | | TGAACACTGCTTGCTTTGTTAGACTGTGTTCAGTAAGTGAATCCCAGTA |
| | | GCCTGAAGCAATGTGTTAGCAGAATCTATTTGTAACATTATTATTGTAC |
| | | AGTAGAATCAATATTAAACACACATGTTTTATTATATGGAGTCATTATT |
| | | TTTAATATGAAATTTAATTTGCAGAGTCCTGAACCTATATAATGGGTTT |
| | | ATTTTAAATGTGATTGTACTTGCAGAATATCTAATTAATTGCTAGGTTA |
| | | ATAACTAAAGAAGCCTATTGCAACAGAGGATGGGCCATGGGGCCTGTGC |
| | | AAGGAAGTATTACCTTCTTATAAATCAAACTAAACATAGCTATTCTCAT |
| | | CTGCATTCCAATGTGATGAAGGCCAAAAATGGCTGGGTGTAGGAGCAGT |
| | | GTCCTCACAATAAAGAGAAGGCATAAGCCTATGCCTAGATAAATCGCGA |
| | | TAGAGCGTTCCTCCTTGTTATCCGGGTCATAGGAAGCTATGATTCTTCC |
| | | CAGTAAGAGAGGCTGTACTGCTTTGGTGACTTCCTACAAAAGGGGAAAA |
| | | ACAGAGATGAGGTTTACACACGGATCCGAATCTTTTGCAGAGAATGGGA |
| | | TAGAGAGCTGGCTTCAAAGAAAAATCCTAAACTCATTAATGCCCTTCGG |
| | | CGATGTTTTTTCTGGAGATTTATGTTCTATGGAATCTTTTTATATTTAG |
| | | GGGTAAGGATCTCATTTGTACATTCATTATGTATCACATAACTATATTC |
| | | ATTTTTGTGATTATGAAAAGACTACGAAATCTGGTGAATAGGTCACACG |
| | | ATGCAGGCTGATAGAATCTTACCTCTTCTAGTTGGCATGCTTTGATGAC |
| | | GCTTCTGTATCTATATTCATCATAGGAAACACCAAAGATGATATTTTCT |
| | | TTAATGGTGCCAGGCATAATCCAGGAAAACTGAGAACAGAATGAAATTC |
| | | TTCCACTGTGCTTAATTTTACCCTCTGAAGGCTCCAGTTCTCCCATTCA |
| | | CAATGGCTTTCCAGGTCGAGAGCATACTAAAAGTGACTCTCTAATTTTC |
| | | TATTTTTGGTAATAGGACATCTCCAAGTTTGCAGAGAAAGACAATATAG |
| | | TTCTTGGAGAAGGTGGAATCACACTGAGTGGAGGTCAACGAGCAAGAAT |
| | | TTCTTTAGCAAGGTGAATAACTAATTATTGGTCTAGCAAGCATTTGCTG |
| | | TCGTATGCCGTCTTCTGCTTG |

Reaction Conditions: The primers were mixed at 500 nM each and 0.6 μl were used in a 10 μl PCR reaction. The final primer concentration was 30 nM. The reaction contained 5 μl of 2× PhoenixTaq PCR master mix (Enzymatics), 1 μl of 10 ng/μl DNA (NA12878, Coriell), 1 μl of 500 mM TMAC, 0.6 μl of 500 nM primer pool, and 2.4 μl of nuclease-free water. The pre-amplification and concatenation PCR conditions were 94° C./5 min, 2 cycles of 94° C./15 sec, 60° C./4 min, 23 cycles of 94° C./15 sec, 72° C./2 min, followed by 20 cycles of 94° C./15 sec, 55° C./1 min, and 72° C./2 min (total PCR: 2 hours, 40 min). 1 μl of pre-amplification and concatenation PCR products were transformed into assembly/tagging PCR with 5 μl of 2× Phoenix Taq master mix, 1 μl of 15 μM T2109-P5-FAM and T2110-P7, and 3 μl of nuclease-free water. PCR cycle conditions were 95° C./5 min, 25 cycles of 95° C./15 sec, 55° C./1 min, and 72° C./2 min. The final PCR products were diluted 1:50 fold and 1 μl was used for CE.

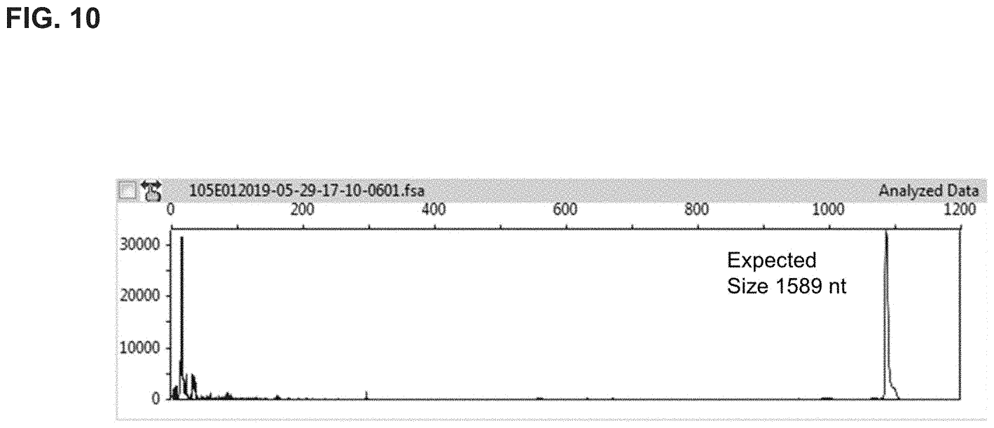

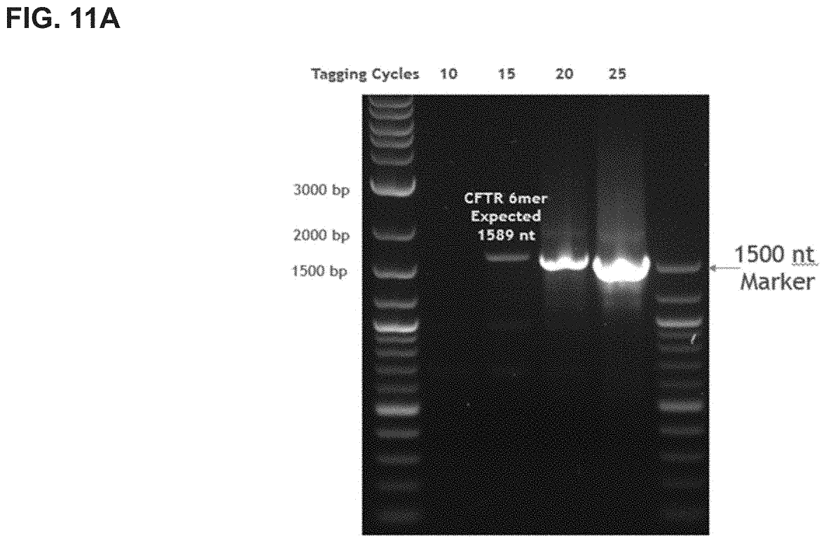

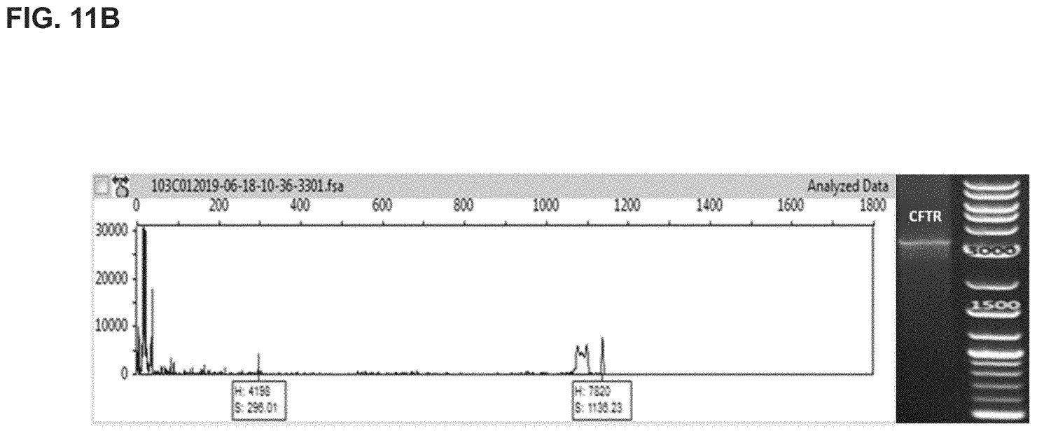

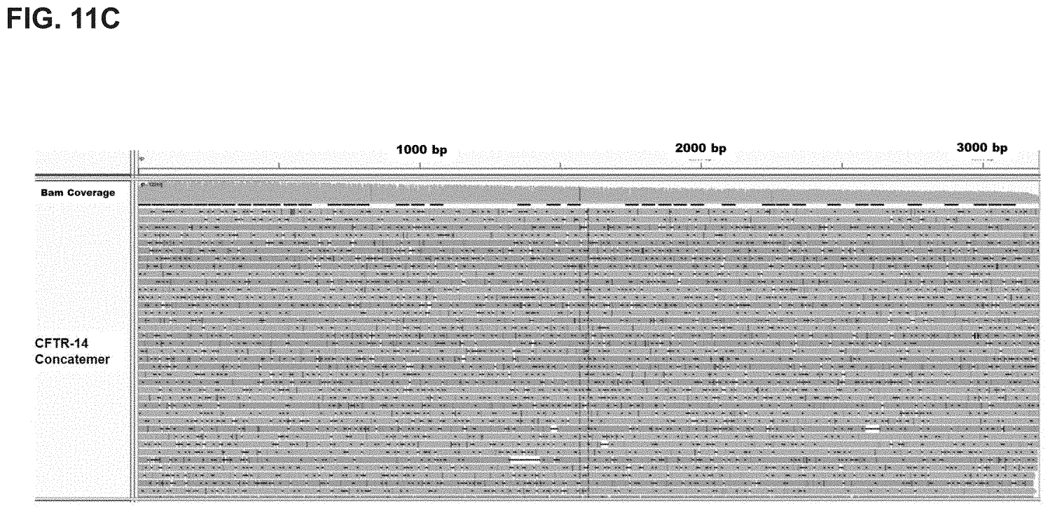

An exemplary CE trace of the concatenated products is shown in FIG. 10. The POP 7 polymer used on CE cannot resolve and size fragments greater than 1000 nt. The 1589 nt constructs therefore showed as about 1086 nt on CE. However, agarose gel analysis confirmed a fragment size of greater than 1500 nt (FIG. 11A).

Nanopore sequencing confirmed the correct 6 amplicon concatenation sequence (1589 nt). 400 fmol of the 6-amplicon concatemer were loaded on a nanopore flow cell of nanopore sequencing. About 100,000 reads were obtained from the concatemer, the majority of which were full length.

The second PCR cycle was also varied by testing at 10, 15, 20, and 25 cycles. Full length products were observed starting at about 15 cycles, but 25 cycles produced the greatest yield (FIG. 11A).

### Example 6

### CFTR Amplicon Concatenation

To test whether it was possible to expand the size and increase the amplicon limit of a multiplex PCR and a concatenation reaction in a single tube, 8 additional CFTR regions of interest (ROIs) were designed and combined with the 6 CFTR amplicons from Example 5 (Table 13). The expected sequence of the assembled 14-amplicon concatenation product is set forth in Table 14.

TABLE 13
CFTR Amplicon Designs for Concatenation.

| | SEQ | |
|---|---|---|
| Primer ID | ID NO | Primer Sequence* |
| T14027_G7-F | 93 | AATGATACGGCGACCACCAactgagacctta |
| | | caccgtttctca |
| T14076_G7-R | 94 | TGCGATGTGCCTGCTATGCTTGatcgcctct |
| | | ccctgctcaga |
| T14077_G8-F | 95 | CAAGCATAGCAGGCACATCGCAatgtcaaag |
| | | atctcacagcaaaataca |
| T14078_G8-R | 96 | GGCCCATCCTCTGTTGCAATaggcttcttta |
| | | gttattaacctagc |
| T14039_G9-F | 97 | ATTGCAACAGAGGATGGGCCatggggcctgt |
| | | gcaagga |
| T14079_G9-R | 98 | TCGGATCCGTGTGTAAACCTCatctctgttt |
| | | ttccccttttgt |
| G14080_G11-F | 99 | GAGGTTTACACACGGATCCGAatcttttgca |
| | | gagaatgggataga |
| G14296_G11-R | 100 | TCTATCAGCCTGCATCGTGTGacctattcac |
| | | cagatttcgtagtc |
| T14297_G10-F | 101 | CACACGATGCAGGCTGATAGAatcttacctc |
| | | ttctagttggcatgct |
| T14298_G10-R | 102 | CGACCTGGAAAGCCATTGTGAatgggagaac |
| | | tggagccttca |
| T14299_G01-F | 103 | TCACAATGGCTTTCCAGGTCGagagcatact |
| | | aaaagtgactctctaattttc |
| T14355_G01-R | 104 | CCTGGCTCCACAACCTAACGacagcaaatgc |
| | | ttgctagacca |
| T14356_G12-F | 105 | CGTTAGGTTGTGGAGCCAGGagagatacttc |
| | | aatagctcagccttc |
| T14357_G12-R | 106 | CCTTGCACAGACCTGTCCAGatgcagcatta |
| | | tggtacattacctg |
| T14358_G13-F | 107 | CTGGACAGGTCTGTGCAAGGagtgggcctct |
| | | tgggaaga |
| T14359_G13-R | 108 | GTGGGTAGGAACGTGCAGACagctcacctgt |
| | | ggtatcactcca |
| T14360_G2-F | 109 | GTCTGCACGTTCCTACCCACatctacactag |
| | | atgaccaggaaatagaga |
| T14351_G2-R | 110 | CGCACCCAGTCGATCTAAGCacatgagcatt |
| | | ataagtaaggtattcaaag |
| T14362_G3-F | 111 | GCTTAGATCGACTGGGTGCGatacagacata |
| | | cttaacggtacttatttttaca |
| T14363_G3-R | 112 | CAGCTGAAGAAGGCACGGTAacaaagatata |
| | | gcaattttggatgacct |
| T14364_G4-F | 113 | TACCGTGCCTTCTTCAGCTGatgaagqaaga |
| | | tgacaaaaatcatttc |
| T14365_G4-R | 114 | CGCATAACTCGTTTCGCCTGatcaggtacaa |
| | | gatattatgaaattacattt |
| T14366_G5-F | 115 | CAGGCGAAACGAGTTATGCGatggagagcat |
| | | accagcagtg |
| T14367_G5-R | 116 | ACTGCTCCATGCGACTGAAAGatctgccaga |
| | | aaaattactaagcac |
| T14368_G6-F | 117 | CTTTCAGTCGCATGGAGCAGTacctatttgc |
| | | tttacagcactcctct |
| T14369_G6-R | 118 | GCAAATCCGGTGTGCCTGATagaacagaatg |
| | | taacattttgtggtgta |
| T14370_G0-F | 119 | ATCAGGCACACCGGATTTGCattaaagctgt |
| | | caagccgtgttc |
| T14371_G0-R | 120 | CAAGCAGAAGACGGCATACAagaaaactccg |
| | | cctttccagt |

*Gene-specific portion of primer in lower case; artificial
tag portion of primer in upper case.

TABLE 14
Assembled Concatenation Product Sequence.

| | SEQ ID NO | Expected Product Sequence |
|---|---|---|
| 14 Amplicons | 121 | AATGATACGGCGACCACCGACTGAGACCTTACACCGTTTCTCATT |
| (Expected | | AGAAGGAGATGCTCCTGTCTCCTGGACAGAAACAAAAAAACAATC |
| concatenation | | TTTTAAACAGACTGGAGAGTTTGGGGAAAAAAGGAAGAATTCTAT |
| product | | TCTCAATCCAATCAACTCTATACGAAAATTTTCCATTGTGCAAAA |
| sequence, | | GACTCCCTTACAAATGAATGGCATCGAAGAGGATTCTGATGAGCC |
| 3203 nt) | | TTTAGAGAGAAGGCTGTCCTTAGTACCAGATTCTGAGCAGGGAGA |
| | | GGCGATCAAGCATAGCAGGCACATCGCAATGTCAAAGATCTCACA |
| | | GCAAAATACACAGAAGGTGGAAATGCCATATTAGAGAACATTTCC |
| | | TTCTCAATAAGTCCTGGCCAGAGGGTGAGATTTGAACACTGCTTG |
| | | CTTTGTTAGACTGTGTTCAGTAAGTGAATCCCAGTAGCCTGAAGC |
| | | AATGTGTTAGCAGAATCTATTTGTAACATTATTATTGTACAGTAG |
| | | AATCAATATTAAACACACATGTTTTATTATATGGAGTCATTATTT |
| | | TTAATATGAAATTTAATTTGCAGAGTCCTGAACCTATATAATGGG |
| | | TTTATTTTAAATGTGATTGTACTTGCAGAATATCTAATTAATTGC |
| | | TAGGTTAATAACTAAAGAAGCCTATTGCAACAGAGGATGGGCCAT |
| | | GGGGCCTGTGCAAGGAAGTATTACCTTCTTATAAATCAAACTAAA |
| | | CATAGCTATTCTCATCTGCATTCCAATGTGATGAAGGCCAAAAAT |
| | | GGCTGGGTGTAGGAGCAGTGTCCTCACAATAAAGAGAAGGCATAA |
| | | GCCTATGCCTAGATAAATCGCGATAGAGCGTTCCTCCTTGTTATC |
| | | CGGGTCATAGGAAGCTATGATTCTTCCCAGTAAGAGAGGCTGTAC |
| | | TGCTTTGGTGACTTCCTACAAAAGGGGAAAAACAGAGATGAGGTT |
| | | TACACACGGATCCGAATCTTTTGCAGAGAATGGGATAGAGAGCTG |
| | | GCTTCAAAGAAAAATCCTAAACTCATTAATGCCCTTCGGCGATGT |
| | | TTTTTCTGGAGATTTATGTTCTATGGAATCTTTTTATATTTAGGG |
| | | GTAAGGATCTCATTTGTACATTCATTATGTATCACATAACTATAT |
| | | TCATTTTTGTGATTATGAAAAGACTACGAAATCTGGTGAATAGGT |
| | | CACACGATGCAGGCTGATAGAATCTTACCTCTTCTAGTTGGCATG |
| | | CTTTGATGACGCTTCTGTATCTATATTCATCATAGGAAACACCAA |
| | | AGATGATATTTTCTTTAATGGTGCCAGGCATAATCCAGGAAAACT |
| | | GAGAACAGAATGAAATTCTTCCACTGTGCTTAATTTTACCCTCTG |
| | | AAGGCTCCAGTTCTCCCATTCACAATGGCTTTCCAGGTCGAGAGC |
| | | ATACTAAAAGTGACTCTCTAATTTTCTATTTTTGGTAATAGGACA |
| | | TCTCCAAGTTTGCAGAGAAAGACAATATAGTTCTTGGAGAAGGTG |
| | | GAATCACACTGAGTGGAGGTCAACGAGCAAGAATTTCTTTAGCAA |
| | | GGTGAATAACTAATTATTGGTCTAGCAAGCATTTGCTGTAGTTAG |
| | | GTTGTGGAGCCAGGAGAGATACTTCAATAGCTCAGCCTTCTTCTT |
| | | CTCAGGGTTCTTTGTGGTGTTTTTATCTGTGCTTCCCTATGCACT |
| | | AATCAAAGGAATCATCCTCCGGAAAATATTCACCACCATCTCATT |
| | | CTGCATTGTTCTGCGCATGGCGGTCACTCGGCAATTTCCCTGGGC |
| | | TGTACAAACATGGTATGACTCTCTTGGAGCAATAAACAAAATACA |
| | | GGTAATGTACCATAATGCTGCATCTGGACAGGTCTGTGCAAGGAG |
| | | TGGGCCTCTTGGGAAGAACTGGATCAGGGAAGAGTACTTTGTTAT |
| | | CAGCTTTTTTGAGACTACTGAACACTGAAGGAGAAATCCAGATCG |
| | | ATGGTGTGTCTTGGGATTCAATAACTTTGCAACAGTGGAGGAAAG |
| | | CCTTTGGAGTGATACCACAGGTGAGCTGTCTGCACGTTCCTACCC |
| | | ACATCTACACTAGATGACCAGGAAATAGAGAGGAAATGTAATTTA |
| | | ATTTCCATTTTCTTTTTAGAGCAGTATACAAAGATGCTGATTTGT |
| | | ATTTATTAGACTCTCCTTTTGGATACCTAGATGTTTTAACAGAAA |
| | | AAGAAATATTTGAAAGGTATGTTCTTTGAATACCTTACTTATAAT |
| | | GCTCATGTGCTTAGATCGACTGGGTGCGATACAGACATACTTAAC |
| | | GGTACTTATTTTTACATACCTGGATGAAGTCAAATATGGTAAGAG |
| | | GCAGAAGGTCATCCAAAATTGCTATATCTTTGTTACCGTGCCTTC |
| | | TTCAGCTGATGAAGAAGATGACAAAAATCATTTCTATTCTCATTT |
| | | GGAACCAGCGCAGTGTTGACAGGTACAAGAACCAGTTGGCAGTAT |
| | | GTAAATTCAGAGCTTTGTGGAACAGAGTTTCAAAGTAAGGCTGCC |
| | | GTCCGAAGGCACGAAGTGTCCATAGTCCTTTTAAGCTTGTAACAA |
| | | GATGAGTGAAAATTGGACTCCTGCCTGTGAAATATTTCCATAGAA |
| | | AACATTGCAAATAACATAAACACAAAATGTAATTTCATAATATCT |
| | | TGTACCTGATCAGGCGAAACGAGTTATGCGATGGAGAGCATACCA |
| | | GCAGTGACTACATGGAACACATACCTTCGATATATTACTGTCCAC |
| | | AAGAGCTTAATTTTTGTGCTAATTTGGTGCTTAGTAATTTTTCTG |
| | | GCAGATCTTTCAGTCGCATGGAGCAGTACCTATTTGCTTTACAGC |
| | | ACTCCTCTTCAAGACAAAGGGAATAGTACTCATAGTAGAAATAAC |
| | | AGCTATGCAGTGATTATCACCAGCACCAGTTCGTATTATGTGTTT |
| | | TACATTTACGTGGGAGTAGCCGACACTTTGCTTGCTATGGGATTC |
| | | TTCAGAGGTCTACCACTGGTGCATACTCTAATCACAGTGTCGAAA |
| | | ATTTTACACCACAAAATGTTACATTCTGTTCTATCAGGCACACCG |
| | | GATTTGCATTAAAGCTGTCAAGCCGTGTTCTAGATAAAATAAGTA |
| | | TTGGACAACTTGTTAGTCTCCTTTCCAACAACCTGAACAAATTTG |
| | | ATGAAGTATGTACCTATTGATTTAATCTTTTAGGCACTATTGTTA |
| | | TAAATTATACAACTGGAAAGGCGGAGTTTTCTTCGTATGCCGTCT |
| | | TCTGCTTG |

Reaction Conditions: The primers were mixed and the final primer concentration was 30 nM. The reaction contained 5 μl of 2× PhoenixTaq PCR master mix (Enzymatics), 1 μl of 10 ng/μl DNA (NA12878, CorieII), 1 μl of 500 mM TMAC, 0.6 μl of 500 nM primer pool, and 2.4 μl of nuclease-free water. The pre-amplification and concatenation PCR conditions were 94° C./5 min, 2 cycles of 94° C./15 sec, 60° C./4 min, 23 cycles of 94° C./15 sec, 72° C./2 min, followed by 20 cycles of 94° C./15 sec. 55° C./1 min, and 72° C./2 min (total PCR: 2 hours, 40 min). 1 μl of pre-amplification and concatenation PCR products were transformed into assembly/tagging PCR with 5 μl of 2× Phoenix Taq master mix, 1 μl of 15 μM T2109-P5-FAM and T2110-P7, and 3 μl of nuclease-free water. PCR cycle conditions were 95° C./5 min, 25 cycles of 95° C./15 sec, 55° C./1 min, and 72° C./2 min. The final PCR products were diluted 1:50 fold and 1 μl was used for CE.

An exemplary CE trace of the concatenated products is shown in FIG. 11B. The POP 7 polymer used on CE cannot resolve and size fragments greater than 1000 nt. The 3203 nt constructs therefore showed as about 1050-1150 nt on CE. However, agarose gel analysis confirmed a fragment size of greater than 3000 nt (FIG. 11B).

Nanopore sequencing confirmed the correct 14 amplicon concatenation sequence (3203 nt). Barcoded CFTR 14-amplicon concatamer was mixed with other samples and sequenced on a nanopore flow cell of nanopore sequencing. After demultiplexing, about 10,000 reads were obtained from the CFTR 14-amplicon concatamer, many of which were full length (FIG. 11C).

### Example 7

### SMN1/SMN2 Copy Number Detection with Multiplex PCR and Concatenation

The amplicon concatenation methods described herein may be applied to co-detection of CFTR variants, and SMN1/SMN2 copy number variation, disease modifiers, and/or silent carrier mutations. To investigate a method of measuring copy number using a spiking external control, the following experiment was performed. A schematic diagram of the experimental design is shown in FIG. 12A.

Briefly, a synthetic gBlock control was designed to contain one modified CFTR amplicon (CFTR* in FIG. 12A, e.g., the 6^{th} CFTR amplicon), a unique restriction site, and a modified SMN* amplicon (i.e., an amplicon of neither SMN1 nor SMN2). Several base changes were made in both the CFTR* and the SMN* sequence in the gBlock. These changes served as stamp mark so that the gBlock control-derived sequence could be differentiated from natural genomic DNA amplification products during subsequent analysis. The gBlock control was cut with the unique restriction enzyme to avoid complications of PCR amplification (for example, to avoid CFTR primer extending over to the SMN*) while maintaining a 1:1 ratio of CFTR* and SMN*. The digested gBlock control was then diluted into low copy number (˜1500 copies/μl) in nucleic acid dilution buffer with 16 ng/μl poly A for long term storage. ˜1500 copies of digested CFTR* and SMN* gBlock control were added into about 10 ng (˜3000 copies) genomic DNA and multiplex overlap extension (MOE) PCR and nanopore sequencing were performed (FIG. 12A).

After nanopore sequencing, counting the sequencing reads as CFTR* with * (with stamp mark from gBlock)=A, CFTR without * (from sample genomic DNA)=B, SMN* with * (with stamp mark from gBlock)=C, SMN1 without * (from sample genomic DNA)=D, and SMN2 without * (from sample genomic DNA)=E, the copy number of SMN1 and SMN2 was calculated as:

SMN1 copy number F=2*(D/C)*(A/B) and SMN2 copy number G=2*(E/C)*(A/B).

The 6 CFTR amplicon and SMN amplicon primers are listed in Table 15. The expected CFTR+SMN amplicon concatenation product sequence and the spiking control gBlock sequence are shown in Table 16. The differential base in the gBlock relative to the natural genomic sequence are boxed in FIG. 12B.

TABLE 15
CFTR + SMN Amplicon Designs for Concatenation.

| | SEQ | |
|---|---|---|
| Primer ID | ID NO | Primer Sequence* |
| T14028_G7-F | 122 | AATGATACGGCGACCACCGActgaga |
| | | ccttacaccgtttctca |
| T14076_G7-R | 123 | TGCGATGTGCCTGCTATGCTTGAtcg |
| | | cctctccctgctcaga |
| T14077_G8-F | 124 | CAAGCATAGCAGGCACATCGCAAtgt |
| | | caaagatctcacagcaaaataca |
| T14078_G8-R | 125 | GGCCCATCCTCTGTTGCAATAggctt |
| | | ctttagttattaacctagc |
| T14039_G9-F | 126 | ATTGCAACAGAGGATGGGCCatgggg |
| | | cctgtgcaagga |
| T14079_G9-R | 127 | TCGGATCCGTGTGTAAACCTCAtctc |
| | | tgtttttccccttttgt |
| T14080_G11-F | 128 | GAGGTTTACACACGGATCCGAAtctt |
| | | ttgcagagaatgggataga |
| T14296_G11-R | 129 | TCTATCAGCCTGCATCGTGTGaccta |
| | | ttcaccagatttcgtagtc |
| T14297_Group10-F | 130 | CACACGATGCAGGCTGATAGAAtctt |
| | | acctcttctagttggcatgct |
| T14298_Group10-R | 131 | CGACCTGGAAAGCCATTGTGAAtggg |
| | | agaactggagccttca |
| T14299_Group01-F | 132 | TCACAATGGCTTTCCAGGTCGAgagc |
| | | atactaaaagtgactctctaattttc |
| T14355_Group01-R | 133 | CCTGGCTCCACAACCTAACGacagca |
| | | aatgcttgctagacca |
| T14634_SMA-F | 134 | CGTTAGGTTGTGGAGCCAGGaacttc |
| | | ctttattttccttacagggt |
| T14638_SMA-M-R | 135 | CAAGCAGAAGACGGCATACGActgct |
| | | ggtctgcctactagtga |

*Gene-specific portion of primer in lower case; artificial
tag portion of primer in upper case.

TABLE 16
Assembled Concatenation Product Sequence.

| | SEQ ID NO | Expected Product Sequence |
|---|---|---|
| 6 CFTR | 136 | AATGATACGGCGACCACCGACTGAGACCTTACACCGTTTCTCATT |
| Amplicons + | | AGAAGGAGATGCTCCTGTCTCCTGGACAGAAACAAAAAAACAATC |
| SMN | | TTTTAAACAGACTGGAGAGTTTGGGGAAAAAAGGAAGAATTCTAT |
| Amplicons | | TCTCAATCCAATCAACTCTATACGAAAATTTTCCATTGTGCAAAA |
| (Expected | | GACTCCCTTACAAATGAATGGCATCGAAGAGGATTCTGATGAGCC |
| size: | | TTTAGAGAGAAGGCTGTCCTTAGTACCAGATTCTGAGCAGGGAGA |
| 1979 nt) | | GGCGATCAAGCATAGCAGGCACATCGCAATGTCAAAGATCTCACA |
| | | GCAAAATACACAGAAGGTGGAAATGCCATATTAGAGAACATTTCC |
| | | TTCTCAATAAGTCCTGGCCAGAGGGTGAGATTTGAACACTGCTTG |
| | | CTTTGTTAGACTGTGTTCAGTAAGTGAATCCCAGTAGCCTGAAGC |
| | | AATGTGTTAGCAGAATCTATTTGTAACATTATTATTGTACAGTAG |
| | | AATCAATATTAAACACACATGTTTTATTATATGGAGTCATTATTT |
| | | TTAATATGAAATTTAATTTGCAGAGTCCTGAACCTATATAATGGG |
| | | TTTATTTTAAATGTGATTGTACTTGCAGAATATCTAATTAATTGC |
| | | TAGGTTAATAACTAAAGAAGCCTATTGCAACAGAGGATGGGCCAT |
| | | GGGGCCTGTGCAAGGAAGTATTACCTTCTTATAAATCAAACTAAA |
| | | CATAGCTATTCTCATCTGCATTCCAATGTGATGAAGGCCAAAAAT |
| | | GGCTGGGTGTAGGAGCAGTGTCCTCACAATAAAGAGAAGGCATAA |
| | | GCCTATGCCTAGATAAATCGCGATAGAGCGTTCCTCCTTGTTATC |
| | | CGGGTCATAGGAAGCTATGATTCTTCCCAGTAAGAGAGGCTGTAC |
| | | TGCTTTGGTGACTTCCTACAAAAGGGGAAAAACAGAGATGAGGTT |
| | | TACACACGGATCCGAATCTTTTGCAGAGAATGGGATAGAGAGCTG |
| | | GCTTCAAAGAAAAATCCTAAACTCATTAATGCCCTTCGGCGATGT |
| | | TTTTTCTGGAGATTTATGTTCTATGGAATCTTTTTATATTTAGGG |
| | | GTAAGGATCTCATTTGTACATTCATTATGTATCACATAACTATAT |
| | | TCATTTTTGTGATTATGAAAAGACTACGAAATCTGGTGAATAGGT |
| | | CACACGATGCAGGCTGATAGAATCTTACCTCTTCTAGTTGGCATG |
| | | CTTTGATGACGCTTCTGTATCTATATTCATCATAGGAAACACCAA |
| | | AGATGATATTTTCTTTAATGGTGCCAGGCATAATCCAGGAAAACT |
| | | GAGAACAGAATGAAATTCTTCCACTGTGCTTAATTTTACCCTCTG |
| | | AAGGCTCCAGTTCTCCCATTCACAATGGCTTTCCAGGTCGAGAGC |
| | | ATACTAAAAGTGACTCTCTAATTTTCTATTTTTGGTAATAGGACA |
| | | TCTCCAAGTTTGCAGAGAAAGACAATATAGTTCTTGGAGAAGGTG |
| | | GAATCACACTGAGTGGAGGTCAACGAGCAAGAATTTCTTTAGCAA |
| | | GGTGAATAACTAATTATTGGTCTAGCAAGCATTTGCTGCGTTAGG |
| | | TTGTGGAGCCAGGAACTTCCTTTATTTTCCTTACAGGGTTTCAGA |
| | | CAAAATCAAAAAGAAGGAAGGTGCTCACATTCCTTAAATTAAGGA |
| | | GTAAGTCTGCCAGCATTATGAAAGTGAATCTTACTTTTGTAAAAC |
| | | TTTATGGTTTGTGGAAAACAAATGTTTTTGAACATTTAAAAAGTT |
| | | CAGATGTTAAAAAGTTGAAAGGTTAATGTAAAACAATCAATATTA |
| | | AAGAATTTTGATGCCAAAACTATTAGATAAAAGGTTAATCTACAT |
| | | CCCTACTAGAATTCTCATACTTAACTGGTTGGTTATGTGGAAGAA |
| | | ACATACTTTCACAATAAAGAGCTTTAGGATATGATGCCATTTTAT |
| | | ATCACTAGTAGGCAGACCAGCAGTCGTATGCCGTCTTCTGCTTG |

Reaction Conditions: The primers were mixed at 250 nM each and 1.2 μl were used in a 10 μl PCR reaction. The final primer concentration was 30 nM. The reaction contained 5 μl of 2× PhoenixTaq PCR master mix (Enzymatics), 1 μl of 10 ng/μl DNA (NA12878, Coriell), 1 μl of diluted HindIII-cut T14641-gBlock (˜1500 copies/μl based on estimate from ng/μl of IDT synthesis label), 1 μl of 500 mM TMAC, 1.2 μl of 250 nM primer pool, and 0.8 μl of nuclease-free water. The pre-amplification and concatenation PCR conditions were 94° C./5 min, 2 cycles of 94° C./15 sec, 60° C./4 min, 23 cycles of 94° C./15 sec, 72° C./2 min, followed by 20 cycles of 94° C./15 sec, 55° C./1 min, and 72° C./2 min (total PCR: 2 hours, 40 min). 1 μl of pre-amplification and concatenation PCR products were transformed into assembly/tagging PCR with 5 μl of 2× Phoenix Taq master mix, 1 μl of 15 μM T2109-P5-FAM and T2110-P7, and 3 μl of nuclease-free water, PCR cycle conditions were 95° C./5 min, 25 cycles of 95° C./15 sec, 55° C./1 min, and 72° C./2 min. The final PCR products were diluted 1:50 fold and 1 μl was used for CE.

An exemplary CE trace of the concatenated products is shown in FIG, 12C. The POP 7 polymer used on CE cannot resolve and size fragments greater than 1000 nt. The 1979 nt constructs therefore showed as about 1077 nt on CE. However, agarose gel analysis confirmed a fragment size of about ˜2000 nt (FIG. 12C).

Genomic DNA samples were spiked in the gBlock control, concatenated, and amplified with a unique sample barcode outside P7 and the P7 tag sequence. These samples were ligated with a nanopore sequencing adaptor and sequenced. The percent (%) of read counts at the differential sites for CFTR*/CFTR, SMN*/SMN1/SMN2 were used to calculate copy number. Nanopore sequencing also confirmed the correct 7 amplicon concatenation sequence (1979 nt).

The sample HG02697 with a SMN1 copy of >4 and a SMN2 copy of 1, as determined by AmplideX® PCR/CE SMN1/2 Kit (RUO), resulted in a SMN1 copy of 4.5 and a SMN2 copy of ˜1. Several other samples with different SMN1/SMN2 ratios were also amplified, concatenated, and barcoded for nanopore sequencing. The concatenation/nanopore sequencing results of observed SMN1/SMN2 ratios were compared with the results determined by AmplideX® PCR/CE SMN1/2 Kit (RUO) (FIG. 12D).

1-159. (canceled)

160. A method of making a library of concatenated amplicons from a target nucleic acid, the method comprising:
i. generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
ii. concatenating the tagged amplicons to generate one or more concatenated amplicons; and
iii. amplifying the one or more concatenated amplicons to generate a library of concatenated amplicons.

161. The method of claim 160, wherein amplifying two or more ROIs comprises polymerase chain reaction (PCR) or isothermal amplification.

162. The method of claim 160, wherein one or more primers in step (i) are depleted prior to concatenating the tagged amplicons.

163. The method of claim 160, wherein one or more primers in step (i) are selected to prevent formation of one or more primer dimers.

164. The method of claim 160, wherein one or more of the primers in step (i) comprise a minimal sequence that is about 6 to about 50 nucleotides in length and is capable of hybridizing to an ROI and also complementary to a sequence in another primer.

165. The method of claim 160, wherein one or more of the primers in step (i) comprise a minimal sequence that is about 30 nucleotides in length and is capable of hybridizing to an ROI and also complementary to a sequence in another primer.

166. The method of claim 160, wherein one or more primers in step (i) are selected to minimize formation of one or more dead-end intermediate products.

167. The method of claim 160, wherein amplifying two or more ROIs comprises PCR, wherein the PCR comprises
magnesium (Mg²⁺) in a concentration of about 0.5 mM to about 4 mM;
dimethyl sulfoxide (DMSO) in a concentration of about 1% to about 8% by volume;
a pH of about 8 to about 10;
wherein each ROI is about 2 to about 10,000 nucleotides in length; and
the concentration of one or more primers is about 1 nM to about 5,000 nM.

168. The method of claim 160, wherein amplifying two or more ROIs comprises PCR, wherein the PCR comprises magnesium (Mg²⁺) in a concentration of about 0.5 mM to about 4 mM.

169. The method of claim 160, wherein amplifying two or more ROIs comprises PCR, wherein the PCR comprises magnesium (Mg²⁺) in a concentration of about 1.5 mM to about 3 mM.

170. The method of claim 160, wherein
one or more primers comprise at least one adenine between the 5′ tag sequence and the sequence capable of hybridizing to the ROI;
one or more primers comprise a 5′ phosphate;
one or more primers comprise a molecular barcode; and/or
the 5′ tag sequence is not homologous to a human genome sequence.

171. The method of claim 160, wherein concatenating the tagged amplicons comprises providing (a) an adjuvant selected from TMAC, ThermaGo, and/or ThermaStop and (b) a DNA polymerase, wherein the DNA polymerase
has 3′ to 5′ exonuclease activity,
is a high-fidelity DNA polymerase, or
is chosen from Q5, Pfu, or Kapa HiFi HotStart DNA polymerase.

172. The method of claim 160, wherein
the one or more tagged amplicons are in a predetermined order resulting from the tag sequences in the primers; and
a) the 5′ tag sequence of the reverse primer for each ROI is complementary to the 5′ tag sequence of the forward primer for the ROI immediately downstream;
b) the order of the one or more concatenated amplicons is identical to the order of the corresponding ROIs in the target nucleic acid; and/or
c) the one or more concatenated amplicons comprise single-copy representation of each tagged amplicon.

173. The method of claim 160, wherein the total length of the one or more concatenated amplicons is about 3,000 to about 4,000 nucleotides.

174. The method of claim 160, wherein the ratio of the one or more concatenated amplicons to the corresponding ROIs in the target nucleic acid is about 1 to 1.

175. The method of claim 160, wherein amplifying the one or more concatenated amplicons comprises a first end primer capable of hybridizing to a tag sequence at the 5′ end of a concatenated amplicon and a second end primer capable of hybridizing to a tag sequence at the 3′ end of a concatenated amplicon, wherein
a) the tag sequence at the 5′ end of the concatenated amplicon is identical to or overlaps with the 5′ tag sequence of a forward primer used to amplify an ROI in and
b) the tag sequence at the 3′ end of the concatenated amplicon is identical to or overlaps with the 5′ tag sequence of a reverse primer used to amplify an ROI

176. The method of claim 160, wherein the first end primer and the second end primer are added in any one of steps (i)-(iii).

177. The method of claim 160 further comprising analyzing the library of concatenated amplicons, by sequencing, gene assembly, and/or structural variation characterization, wherein
a) sequencing comprises single-molecule sequencing; long-read sequencing; or sequencing about 800 nucleotides or longer;
b) sequencing comprises nanopore sequencing or single-molecule real-time (SMRT) sequencing;
c) structural variation characterization comprises detecting or quantifying single nucleotide variants (SNV), repeat sequences, indels, gene chimera, and/or gene copy number;
d) detecting or quantifying gene copy number comprises detecting or quantifying one or more molecular barcodes;
e) detecting or quantifying gene copy number comprises comparing to an external spiking control;
f) detecting or quantifying gene copy number comprises comparing to an external spiking control, where the external spiking control comprises a synthetic gBlock control, or
g) the structural variation characterization comprises labeling and/or direct imaging.

178. The method of claim 160, wherein the target nucleic acid comprises one or more genes chosen from KRAS, BRAF, PIK3C, EGFR, ERBB2, FMR1, HBA1, HBA2, GBA, CFTR, IKBKAP, ABCC8, FANCC, GALT, G6PC, HBB, BLM, ASPA, TMEM216, BCKDHA, BCKDHB, ACADM, MCOLN1, NEB, SMPD1, F8, HEXA, PCDH15, DMD, CYP21A2, and CLRN1.

179. The method of claim 160, wherein the target nucleic acid is in a sample chosen from:
a blood sample;
a buccal sample;
a biopsy sample;
a frozen tissue or formalin-fixed paraffin-embedded (FFPE) tissue;
an extracellular sample;
a liquid biopsy sample; or
cell-free DNA or DNA from circulating tumor cells.

180. The method of claim 160, wherein making a library of concatenated amplicons from the target nucleic acid comprises amplifying the one or more concatenated amplicons by PCR to generate a library of concatenated amplicons, wherein the PCR comprises
synthesizing about 2-20 amplicons,
synthesizing a concatenated amplicon of about 1,000-5,000 nucleotides,
a concentration of one or more primers of about 30 nM.
a primer artificial tag, and/or
an enzyme that lacks 3′ to 5′ proofreading activity.

181. The method of claim 180, wherein the PCR comprises a concentration of dimethyl sulfoxide (DMSO) of about 1% to about 8% by volume.

182. A method of making a library of concatenated amplicons from a target nucleic acid, the method comprising:
generating tagged amplicons by amplifying two or more regions of interest (ROIs) from the target nucleic acid, wherein each ROI is amplified with a forward primer and a reverse primer, wherein each primer comprises a 5′ tag sequence and a sequence capable of hybridizing to the ROI, and wherein the 5′ tag sequence of the reverse primer for each ROI is complementary to the 5′ tag sequence of the forward primer for another ROI;
concatenating the tagged amplicons to generate one or more concatenated amplicons; and
amplifying the one or more concatenated amplicons by PCR to generate a library of concatenated amplicons,
wherein the PCR comprises
magnesium in a concentration of about 1.5 mM to about 3 mM;
DMSO in a concentration of about 3% to about 6% by volume;
a concentration of one or more primers of about 30 nM; and
a pH of about 8.5 to about 9.2.

183. The method of claim 182,
wherein one or more primers in comprise a minimal sequence of about 6 to about 50 nucleotides in length that is capable of hybridizing to an ROI and also complementary to a sequence in another primer; and
wherein the method further comprises concatenating at least two tagged amplicons; and
wherein each tagged amplicon is about 50 to about 10,000 nucleotides in length; and
the total length of the one or more concatenated amplicons is about 2,000 to about 5,000 nucleotides.

184. The method of claim 183, wherein the minimal sequence is about 15 to about 30 nucleotides in length.

185. The method of claim 182, wherein one or more primers are selected to minimize formation of one or more dead-end intermediate products that cannot form one or more concatenated amplicons.

186. A library of concatenated amplicons prepared according to the method of claim 160.

187. A method of selecting a set of primers capable of amplifying two or more regions of interest (ROIs) from a target nucleic acid, comprising selecting a forward primer and a reverse primer for each ROI, wherein each primer comprises a 5′ tag sequence and a sequence capable of hybridizing to the ROI, and wherein:
a) the 5′ tag sequence of the reverse primer for each ROI is complementary to the 5′ tag sequence of the forward primer for another ROI;
b) the 5′ tag sequence is an artificial tag sequence; and
c) each primer comprises a minimal sequence that is capable of hybridizing to an ROI and is also complementary to a sequence in another primer.

188. A method of sequencing a target nucleic acid, comprising generating a library of concatenated amplicons of the target nucleic acid according to the method of claim 160, and sequencing the library.

189. A kit comprising a set of primers and instructions for using the primers in generating a library of concatenated amplicons of a target nucleic acid according to the method of claim 160.


SEQUENCE LISTING (151 sequences)

SEQ ID NO: 1 - DNA, 20 bp, Artificial Sequence
  Description of Artificial Sequence Synthetic primer tag
aatgatacggcgaccaccga

SEQ ID NO: 2 - DNA, 21 bp, Artificial Sequence
  Description of Artificial Sequence Synthetic primer tag
caagcagaagacggcatacga

SEQ ID NO: 3 - DNA, 20 bp, Artificial Sequence
  Description of Artificial Sequence Synthetic primer tag
aggactggggttttattata

SEQ ID NO: 4 - DNA, 10 bp, Artificial Sequence
  Description of Artificial Sequence Synthetic primer tag
ttttattata

SEQ ID NO: 5 - DNA, 40 bp, Artificial Sequence
  Description of Artificial Sequence Synthetic primer
aatgatacggcgaccaccgactgtatcgtcaaggcactct

SEQ ID NO: 6 - DNA, 40 bp, Artificial Sequence
  Description of Artificial Sequence Synthetic primer
aggactggggttttattataaggcctgctgaaaatgactg

SEQ ID NO: 7 - DNA, 40 bp, Artificial Sequence
  Description of Artificial Sequence Synthetic primer
tataataaaaccccagtcctcatgtactggtccctcattg

SEQ ID NO: 8 - DNA, 44 bp, Artificial Sequence
  Description of Artificial Sequence Synthetic primer
gtaagaattgaggctagtaattgatggagaaacctgtctcttgg

SEQ ID NO: 9 - DNA, 46 bp, Artificial Sequence
  Description of Artificial Sequence Synthetic primer
tcaattactagcctcaattcttaccatccacaaaatggatccagac

SEQ ID NO: 10 - DNA, 44 bp, Artificial Sequence
  Description of Artificial Sequence Synthetic primer
aatctgcccatcctcagatatatttcttcatgaagacctcacag

SEQ ID NO: 11 - DNA, 41 bp, Artificial Sequence
  Description of Artificial Sequence Synthetic primer
tatctgaggatgggcagattacagtgggacaaagaattgga

SEQ ID NO: 12 - DNA, 45 bp, Artificial Sequence
  Description of Artificial Sequence Synthetic primer
tttgagctgtacaatgtcaccacattacatacttaccatgccact

SEQ ID NO: 13 - DNA, 40 bp, Artificial Sequence
  Description of Artificial Sequence Synthetic primer
gtgacattgtacagctcaaagcaatttctacacgagatcc

SEQ ID NO: 14 - DNA, 42 bp, Artificial Sequence
  Description of Artificial Sequence Synthetic primer
tttatctaaggcatctccattttagcacttacctgtgactcc

SEQ ID NO: 15 - DNA, 41 bp, Artificial Sequence
  Description of Artificial Sequence Synthetic primer
aaatggagatgccttagataaaactgagcaagaggctttgg

SEQ ID NO: 16 - DNA, 40 bp, Artificial Sequence
  Description of Artificial Sequence Synthetic primer
tttttccagtgaagatccaatccatttttgttgtccagcc

SEQ ID NO: 17 - DNA, 39 bp, Artificial Sequence
  Description of Artificial Sequence Synthetic primer
ttggatcttcactggaaaaaactgtttgggacctccggt

SEQ ID NO: 18 - DNA, 42 bp, Artificial Sequence
  Description of Artificial Sequence Synthetic primer
ttggttggaaagcggtgacttactgcagctgttttcacctct

SEQ ID NO: 19 - DNA, 38 bp, Artificial Sequence
  Description of Artificial Sequence Synthetic primer
caccgctttccaaccaagctctcttgaggatcttgaag

SEQ ID NO: 20 - DNA, 39 bp, Artificial Sequence
  Description of Artificial Sequence Synthetic primer
gtccctatgagggaccttaccttatacaccgtgccgaac

SEQ ID NO: 21 - DNA, 39 bp, Artificial Sequence
  Description of Artificial Sequence Synthetic primer
taaggtccctcatagggactctggatcccagaaggtgag

SEQ ID NO: 22 - DNA, 36 bp, Artificial Sequence
  Description of Artificial Sequence Synthetic primer
gggagggaacctccacacagcaaagcagaaactcac

SEQ ID NO: 23 - DNA, 35 bp, Artificial Sequence
  Description of Artificial Sequence Synthetic primer
tggaggttccctccctccaggaagcctacgtgatg

SEQ ID NO: 24 - DNA, 39 bp, Artificial Sequence
  Description of Artificial Sequence Synthetic primer
tcctggctgattgtctttgtgttcccggacatagtccag

SEQ ID NO: 25 - DNA, 39 bp, Artificial Sequence
  Description of Artificial Sequence Synthetic primer
caaagacaatcagccaggaacgtactggtgaaaacaccg

SEQ ID NO: 26 - DNA, 37 bp, Artificial Sequence
  Description of Artificial Sequence Synthetic primer
aagggtacgcatggtattctttctcttccgcacccag

SEQ ID NO: 27 - DNA, 37 bp, Artificial Sequence
  Description of Artificial Sequence Synthetic primer
aataccatgcgtacccttgtccccaggaagcatacgt

SEQ ID NO: 28 - DNA, 39 bp, Artificial Sequence
  Description of Artificial Sequence Synthetic primer
caagcagaagacggcatacgacaccgtggatgtcaggca

SEQ ID NO: 29 - DNA, 649 bp, Artificial Sequence
  Description of Artificial Sequence Synthetic polynucleotide
aatgatacggcgaccaccgactgtatcgtcaaggcactcttgcctacgccaccagctccaactaccacaagtttatattcagtcattttcagcaggccttataataaaaccccagtcctcatgtactggtccctcattgcactgtactcctcttgacctgctgtgtcgagaatatccaagagacaggtttctccatcaattactagcctcaattcttaccatccacaaaatggatccagacaactgttcaaactgatgggacccactccatcgagatttcactgtagctagaccaaaatcacctatttttactgtgaggtcttcatgaagaaatatatctgaggatgggcagattacagtgggacaaagaattggatctggatcatttggaacagtctacaagggaaagtggcatggtaagtatgtaatgtggtgacattgtacagctcaaagcaatttctacacgagatcctctctctgaaatcactgagcaggagaaagattttctatggagtcacaggtaagtgctaaaatggagatgccttagataaaactgagcaagaggctttggagtatttcatgaaacaaatgaatgatgcacatcatggtggctggacaacaaaaatggattggatcttcactggaaaaa

SEQ ID NO: 30 - DNA, 692 bp, Artificial Sequence
  Description of Artificial Sequence Synthetic polynucleotide
ttggatcttcactggaaaaaactgtttgggacctccggtcagaaaaccaaaattataagcaacagaggtgaaaacagctgcagtaagtcaccgctttccaaccaagctctcttgaggatcttgaaggaaactgaattcaaaaagatcaaagtgctgggctccggtgcgttcggcacggtgtataaggtaaggtccctcatagggactctggatcccagaaggtgagaaagttaaaattcccgtcgctatcaaggaattaagagaagcaacatctccgaaagccaacaaggaaatcctcgatgtgagtttctgctttgctgtgtggaggttccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatcagccaggaacgtactggtgaaaacaccgcagcatgtcaagatcacagattttgggctggccaaactgctgggtgcggaagagaaagaataccatgcgtacccttgtccccaggaagcatacgtgatggctggtgtgggctccccatatgtctcccgccttctgggcatctgcctgacatccacggtgtcgtatgccgtcttctgcttg

SEQ ID NO: 31 - DNA, 1321 bp, Artificial Sequence
  Description of Artificial Sequence Synthetic polynucleotide
aatgatacggcgaccaccgactgtatcgtcaaggcactcttgcctacgccaccagctccaactaccacaagtttatattcagtcattttcagcaggccttataataaaaccccagtcctcatgtactggtccctcattgcactgtactcctcttgacctgctgtgtcgagaatatccaagagacaggtttctccatcaattactagcctcaattcttaccatccacaaaatggatccagacaactgttcaaactgatgggacccactccatcgagatttcactgtagctagaccaaaatcacctatttttactgtgaggtcttcatgaagaaatatatctgaggatgggcagattacagtgggacaaagaattggatctggatcatttggaacagtctacaagggaaagtggcatggtaagtatgtaatgtggtgacattgtacagctcaaagcaatttctacacgagatcctctctctgaaatcactgagcaggagaaagattttctatggagtcacaggtaagtgctaaaatggagatgccttagataaaactgagcaagaggctttggagtatttcatgaaacaaatgaatgatgcacatcatggtggctggacaacaaaaatggattggatcttcactggaaaaaactgtttgggacctccggtcagaaaaccaaaattataagcaacagaggtgaaaacagctgcagtaagtcaccgctttccaaccaagctctcttgaggatcttgaaggaaactgaattcaaaaagatcaaagtgctgggctccggtgcgttcggcacggtgtataaggtaaggtccctcatagggactctggatcccagaaggtgagaaagttaaaattcccgtcgctatcaaggaattaagagaagcaacatctccgaaagccaacaaggaaatcctcgatgtgagtttctgctttgctgtgtggaggttccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatcagccaggaacgtactggtgaaaacaccgcagcatgtcaagatcacagattttgggctggccaaactgctgggtgcggaagagaaagaataccatgcgtacccttgtccccaggaagcatacgtgatggctggtgtgggctccccatatgtctcccgccttctgggcatctgcctgacatccacggtgtcgtatgccgtcttctgcttg

SEQ ID NO: 32 - DNA, 40 bp, Artificial Sequence; identical to SEQ ID NO: 5
  Description of Artificial Sequence Synthetic primer

SEQ ID NO: 33 - DNA, 40 bp, Artificial Sequence
  Description of Artificial Sequence Synthetic primer
cctggctccacaacctaacgaggcctgctgaaaatgactg

SEQ ID NO: 34 - DNA, 40 bp, Artificial Sequence
  Description of Artificial Sequence Synthetic primer
cgttaggttgtggagccaggcatgtactggtccctcattg

SEQ ID NO: 35 - DNA, 40 bp, Artificial Sequence
  Description of Artificial Sequence Synthetic primer
ccttgcacagacctgtccagtggagaaacctgtctcttgg

SEQ ID NO: 36 - DNA, 42 bp, Artificial Sequence
  Description of Artificial Sequence Synthetic primer
ctggacaggtctgtgcaaggcatccacaaaatggatccagac

SEQ ID NO: 37 - DNA, 44 bp, Artificial Sequence
  Description of Artificial Sequence Synthetic primer
gtgggtaggaacgtgcagactatttcttcatgaagacctcacag

SEQ ID NO: 38 - DNA, 41 bp, Artificial Sequence
  Description of Artificial Sequence Synthetic primer
gtctgcacgttcctacccacacagtgggacaaagaattgga

SEQ ID NO: 39 - DNA, 45 bp, Artificial Sequence
  Description of Artificial Sequence Synthetic primer
cgcacccagtcgatctaagccacattacatacttaccatgccact

SEQ ID NO: 40 - DNA, 40 bp, Artificial Sequence
  Description of Artificial Sequence Synthetic primer
gcttagatcgactgggtgcggcaatttctacacgagatcc

SEQ ID NO: 41 - DNA, 40 bp, Artificial Sequence
  Description of Artificial Sequence Synthetic primer
cagctgaagaaggcacggtatagcacttacctgtgactcc

SEQ ID NO: 42 - DNA, 39 bp, Artificial Sequence
  Description of Artificial Sequence Synthetic primer
taccgtgccttcttcagctgactgagcaagaggctttgg

SEQ ID NO: 43 - DNA, 40 bp, Artificial Sequence
  Description of Artificial Sequence Synthetic primer
cgcataactcgtttcgcctgtccatttttgttgtccagcc

SEQ ID NO: 44 - DNA, 39 bp, Artificial Sequence
  Description of Artificial Sequence Synthetic primer
caggcgaaacgagttatgcgactgtttgggacctccggt

SEQ ID NO: 45 - DNA, 45 bp, Artificial Sequence
  Description of Artificial Sequence Synthetic primer
ggcccatcctctgttgcaatacttactgcagctgttttcacctct

SEQ ID NO: 46 - DNA, 41 bp, Artificial Sequence
  Description of Artificial Sequence Synthetic primer
attgcaacagaggatgggccgctctcttgaggatcttgaag

SEQ ID NO: 47 - DNA, 41 bp, Artificial Sequence
  Description of Artificial Sequence Synthetic primer
tcggatccgtgtgtaaacctcccttatacaccgtgccgaac

SEQ ID NO: 48 - DNA, 42 bp, Artificial Sequence
  Description of Artificial Sequence Synthetic primer
gaggtttacacacggatccgaagactctggatcccagaaggt

SEQ ID NO: 49 - DNA, 43 bp, Artificial Sequence
  Description of Artificial Sequence Synthetic primer
tctatcagcctgcatcgtgtgacacagcaaagcagaaactcac

SEQ ID NO: 50 - DNA, 41 bp, Artificial Sequence
  Description of Artificial Sequence Synthetic primer
cacacgatgcaggctgatagatccaggaagcctacgtgatg

SEQ ID NO: 51 - DNA, 41 bp, Artificial Sequence
  Description of Artificial Sequence Synthetic primer
cgacctggaaagccattgtgatgttcccggacatagtccag

SEQ ID NO: 52 - DNA, 41 bp, Artificial Sequence
  Description of Artificial Sequence Synthetic primer
tcacaatggctttccaggtcgacgtactggtgaaaacaccg

SEQ ID NO: 53 - DNA, 40 bp, Artificial Sequence
  Description of Artificial Sequence Synthetic primer
actgctccatgcgactgaaagctttctcttccgcacccag

SEQ ID NO: 54 - DNA, 40 bp, Artificial Sequence
  Description of Artificial Sequence Synthetic primer
ctttcagtcgcatggagcagtgtccccaggaagcatacgt

SEQ ID NO: 55 - DNA, 39 bp, Artificial Sequence; identical to SEQ ID NO: 28
  Description of Artificial Sequence Synthetic primer

SEQ ID NO: 56 - DNA, 39 bp, Artificial Sequence
  Description of Artificial Sequence Synthetic primer
cacacgatgcaggctgatagaaccatgcgaagccacact

SEQ ID NO: 57 - DNA, 45 bp, Artificial Sequence
  Description of Artificial Sequence Synthetic primer
actgctccatgcgactgaaagactgcatggtattctttctcttcc

SEQ ID NO: 58 - DNA, 43 bp, Artificial Sequence
  Description of Artificial Sequence Synthetic primer
aatgatacggcgaccaccgactgagaccttacaccgtttctca

SEQ ID NO: 59 - DNA, 41 bp, Artificial Sequence
  Description of Artificial Sequence Synthetic primer
tgcgatgtgcctgctatgcttgtcgcctctccctgctcaga

SEQ ID NO: 60 - DNA, 48 bp, Artificial Sequence
  Description of Artificial Sequence Synthetic primer
caagcatagcaggcacatcgcatgtcaaagatctcacagcaaaataca

SEQ ID NO: 61 - DNA, 44 bp, Artificial Sequence
  Description of Artificial Sequence Synthetic primer
ggcccatcctctgttgcaatggcttctttagttattaacctagc

SEQ ID NO: 62 - DNA, 38 bp, Artificial Sequence
  Description of Artificial Sequence Synthetic primer
attgcaacagaggatgggccatggggcctgtgcaagga

SEQ ID NO: 63 - DNA, 42 bp, Artificial Sequence
  Description of Artificial Sequence Synthetic primer
tcggatccgtgtgtaaacctctctctgtttttccccttttgt

SEQ ID NO: 64 - DNA, 44 bp, Artificial Sequence
  Description of Artificial Sequence Synthetic primer
gaggtttacacacggatccgatcttttgcagagaatgggataga

SEQ ID NO: 65 - DNA, 45 bp, Artificial Sequence
  Description of Artificial Sequence Synthetic primer
caagcagaagacggcatacgaacctattcaccagatttcgtagtc

SEQ ID NO: 66 - DNA, 20 bp, Artificial Sequence
  Description of Artificial Sequence Synthetic primer
aatgatacggcgaccaccga

SEQ ID NO: 67 - DNA, 21 bp, Artificial Sequence
  Description of Artificial Sequence Synthetic primer
caagcagaagacggcatacga

SEQ ID NO: 68 - DNA, 1186 bp, Artificial Sequence
  Description of Artificial Sequence Synthetic polynucleotide
aatgatacggcgaccaccgactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgacaagcatagcaggcacatcgcaagtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattgcaacagaggatgggccatggggcctgtgcaaggaagtattaccttcttataaatcaaactaaacatagctattctcatctgcattccaatgtgatgaaggccaaaaatggctgggtgtaggagcagtgtcctcacaataaagagaaggcataagcctatgcctagataaatcgcgatagagcgttcctccttgttatccgggtcataggaagctatgattcttcccagtaagagaggctgtactgctttggtgacttcctacaaaaggggaaaaacagagagaggtttacacacggatccgatcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggttcgtatgccgtcttctgcttg

SEQ ID NO: 69 - DNA, 43 bp, Artificial Sequence; identical to SEQ ID NO: 58
  Description of Artificial Sequence Synthetic primer

SEQ ID NO: 70 - DNA, 42 bp, Artificial Sequence
  Description of Artificial Sequence Synthetic primer
tgcgatgtgcctgctatgcttgatcgcctctccctgctcaga

SEQ ID NO: 71 - DNA, 49 bp, Artificial Sequence
  Description of Artificial Sequence Synthetic primer
caagcatagcaggcacatcgcaatgtcaaagatctcacagcaaaataca

SEQ ID NO: 72 - DNA, 45 bp, Artificial Sequence
  Description of Artificial Sequence Synthetic primer
ggcccatcctctgttgcaataggcttctttagttattaacctagc

SEQ ID NO: 73 - DNA, 38 bp, Artificial Sequence; identical to SEQ ID NO: 62
  Description of Artificial Sequence Synthetic primer

SEQ ID NO: 74 - DNA, 43 bp, Artificial Sequence
  Description of Artificial Sequence Synthetic primer
tcggatccgtgtgtaaacctcatctctgtttttccccttttgt

SEQ ID NO: 75 - DNA, 45 bp, Artificial Sequence
  Description of Artificial Sequence Synthetic primer
gaggtttacacacggatccgaatcttttgcagagaatgggataga

SEQ ID NO: 76 - DNA, 45 bp, Artificial Sequence; identical to SEQ ID NO: 65
  Description of Artificial Sequence Synthetic primer

SEQ ID NO: 77 - DNA, 43 bp, Artificial Sequence; identical to SEQ ID NO: 58
  Description of Artificial Sequence Synthetic primer

SEQ ID NO: 78 - DNA, 42 bp, Artificial Sequence; identical to SEQ ID NO: 70
  Description of Artificial Sequence Synthetic primer

SEQ ID NO: 79 - DNA, 1191 bp, Artificial Sequence
  Description of Artificial Sequence Synthetic polynucleotide
aatgatacggcgaccaccgactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatcaagcatagcaggcacatcgcaatgtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagcctattgcaacagaggatgggccatggggcctgtgcaaggaagtattaccttcttataaatcaaactaaacatagctattctcatctgcattccaatgtgatgaaggccaaaaatggctgggtgtaggagcagtgtcctcacaataaagagaaggcataagcctatgcctagataaatcgcgatagagcgttcctccttgttatccgggtcataggaagctatgattcttcccagtaagagaggctgtactgctttggtgacttcctacaaaaggggaaaaacagagatgaggtttacacacggatccgaatcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggttcgtatgccgtcttctgcttg

SEQ ID NO: 80 - DNA, 43 bp, Artificial Sequence; identical to SEQ ID NO: 58
  Description of Artificial Sequence Synthetic primer

SEQ ID NO: 81 - DNA, 42 bp, Artificial Sequence; identical to SEQ ID NO: 70
  Description of Artificial Sequence Synthetic primer

SEQ ID NO: 82 - DNA, 49 bp, Artificial Sequence; identical to SEQ ID NO: 71
  Description of Artificial Sequence Synthetic primer

SEQ ID NO: 83 - DNA, 45 bp, Artificial Sequence; identical to SEQ ID NO: 72
  Description of Artificial Sequence Synthetic primer

SEQ ID NO: 84 - DNA, 38 bp, Artificial Sequence; identical to SEQ ID NO: 62
  Description of Artificial Sequence Synthetic primer

SEQ ID NO: 85 - DNA, 43 bp, Artificial Sequence; identical to SEQ ID NO: 74
  Description of Artificial Sequence Synthetic primer

SEQ ID NO: 86 - DNA, 45 bp, Artificial Sequence; identical to SEQ ID NO: 75
  Description of Artificial Sequence Synthetic primer

SEQ ID NO: 87 - DNA, 45 bp, Artificial Sequence
  Description of Artificial Sequence Synthetic primer
tctatcagcctgcatcgtgtgacctattcaccagatttcgtagtc

SEQ ID NO: 88 - DNA, 47 bp, Artificial Sequence
  Description of Artificial Sequence Synthetic primer
cacacgatgcaggctgatagaatcttacctcttctagttggcatgct

SEQ ID NO: 89 - DNA, 42 bp, Artificial Sequence
  Description of Artificial Sequence Synthetic primer
cgacctggaaagccattgtgaatgggagaactggagccttca

SEQ ID NO: 90 - DNA, 52 bp, Artificial Sequence
  Description of Artificial Sequence Synthetic primer
tcacaatggctttccaggtcgagagcatactaaaagtgactctctaattttc

SEQ ID NO: 91 - DNA, 42 bp, Artificial Sequence
  Description of Artificial Sequence Synthetic primer
caagcagaagacggcatacgacagcaaatgcttgctagacca

SEQ ID NO: 92 - DNA, 1589 bp, Artificial Sequence
  Description of Artificial Sequence Synthetic polynucleotide
aatgatacggcgaccaccgactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatcaagcatagcaggcacatcgcaatgtcaaagatctcacagcaaaatacacagaaggtggaaatgccatattagagaacatttccatctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagcctattgcaacagaggatgggccatggggcctgtgcaaggaagtattaccttcttataaatcaaactaaacatagctattctcatctgcattccaatgtgatgaaggccaaaaatggctgggtgtaggagcagtgtcctcacaataaagagaaggcataagcctatgcctagataaatcgcgatagagcgttcctccttgttatccgggtcataggaagctatgattcttcccagtaagagaggctgtactgctttggtgacttcctacaaaaggggaaaaacagagatgaggtttacacacggatccgaatcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggtcacacgatgcaggctgatagaatcttacctcttctagttggcatgctttgatgacgcttctgtatctatattcatcataggaaacaccaaagatgatattttctttaatggtgccaggcataatccaggaaaactgagaacagaatgaaattcttccactgtgcttaattttaccctctgaaggctccagttctcccattcacaatggctttccaggtcgagagcatactaaaagtgactctctaattttctatttttggtaataggacatctccaagtttgcagagaaagacaatatagttcttggagaaggtggaatcacactgagtggaggtcaacgagcaagaatttctttagcaaggtgaataactaattattggtctagcaagcatttgctgtcgtatgccgtcttctgcttg

SEQ ID NO: 93 - DNA, 43 bp, Artificial Sequence; identical to SEQ ID NO: 58
  Description of Artificial Sequence Synthetic primer

SEQ ID NO: 94 - DNA, 42 bp, Artificial Sequence; identical to SEQ ID NO: 70
  Description of Artificial Sequence Synthetic primer

SEQ ID NO: 95 - DNA, 49 bp, Artificial Sequence; identical to SEQ ID NO: 71
  Description of Artificial Sequence Synthetic primer

SEQ ID NO: 96 - DNA, 45 bp, Artificial Sequence; identical to SEQ ID NO: 72
  Description of Artificial Sequence Synthetic primer

SEQ ID NO: 97 - DNA, 38 bp, Artificial Sequence; identical to SEQ ID NO: 62
  Description of Artificial Sequence Synthetic primer

SEQ ID NO: 98 - DNA, 43 bp, Artificial Sequence; identical to SEQ ID NO: 74
  Description of Artificial Sequence Synthetic primer

SEQ ID NO: 99 - DNA, 45 bp, Artificial Sequence; identical to SEQ ID NO: 75
  Description of Artificial Sequence Synthetic primer

SEQ ID NO: 100 - DNA, 45 bp, Artificial Sequence; identical to SEQ ID NO: 87
  Description of Artificial Sequence Synthetic primer

SEQ ID NO: 101 - DNA, 47 bp, Artificial Sequence; identical to SEQ ID NO: 88
  Description of Artificial Sequence Synthetic primer

SEQ ID NO: 102 - DNA, 42 bp, Artificial Sequence; identical to SEQ ID NO: 89
  Description of Artificial Sequence Synthetic primer

SEQ ID NO: 103 - DNA, 52 bp, Artificial Sequence; identical to SEQ ID NO: 90
  Description of Artificial Sequence Synthetic primer

SEQ ID NO: 104 - DNA, 42 bp, Artificial Sequence
  Description of Artificial Sequence Synthetic primer
cctggctccacaacctaacgacagcaaatgcttgctagacca

SEQ ID NO: 105 - DNA, 46 bp, Artificial Sequence
  Description of Artificial Sequence Synthetic primer
cgttaggttgtggagccaggagagatacttcaatagctcagccttc

SEQ ID NO: 106 - DNA, 45 bp, Artificial Sequence
  Description of Artificial Sequence Synthetic primer
ccttgcacagacctgtccagatgcagcattatggtacattacctg

SEQ ID NO: 107 - DNA, 39 bp, Artificial Sequence
  Description of Artificial Sequence Synthetic primer
ctggacaggtctgtgcaaggagtgggcctcttgggaaga

SEQ ID NO: 108 - DNA, 43 bp, Artificial Sequence
  Description of Artificial Sequence Synthetic primer
gtgggtaggaacgtgcagacagctcacctgtggtatcactcca

SEQ ID NO: 109 - DNA, 49 bp, Artificial Sequence
  Description of Artificial Sequence Synthetic primer
gtctgcacgttcctacccacatctacactagatgaccaggaaatagaga

SEQ ID NO: 110 - DNA, 50 bp, Artificial Sequence
  Description of Artificial Sequence Synthetic primer
cgcacccagtcgatctaagcacatgagcattataagtaaggtattcaaag

SEQ ID NO: 111 - DNA, 53 bp, Artificial Sequence
  Description of Artificial Sequence Synthetic primer
gcttagatcgactgggtgcgatacagacatacttaacggtacttatttttaca

SEQ ID NO: 112 - DNA, 48 bp, Artificial Sequence
  Description of Artificial Sequence Synthetic primer
cagctgaagaaggcacggtaacaaagatatagcaattttggatgacct

SEQ ID NO: 113 - DNA, 46 bp, Artificial Sequence
  Description of Artificial Sequence Synthetic primer
taccgtgccttcttcagctgatgaagaagatgacaaaaatcatttc

SEQ ID NO: 114 - DNA, 51 bp, Artificial Sequence
  Description of Artificial Sequence Synthetic primer
cgcataactcgtttcgcctgatcaggtacaagatattatgaaattacattt

SEQ ID NO: 115 - DNA, 41 bp, Artificial Sequence
  Description of Artificial Sequence Synthetic primer
caggcgaaacgagttatgcgatggagagcataccagcagtg

SEQ ID NO: 116 - DNA, 46 bp, Artificial Sequence
  Description of Artificial Sequence Synthetic primer
actgctccatgcgactgaaagatctgccagaaaaattactaagcac

SEQ ID NO: 117 - DNA, 47 bp, Artificial Sequence
  Description of Artificial Sequence Synthetic primer
ctttcagtcgcatggagcagtacctatttgctttacagcactcctct

SEQ ID NO: 118 - DNA, 48 bp, Artificial Sequence
  Description of Artificial Sequence Synthetic primer
gcaaatccggtgtgcctgatagaacagaatgtaacattttgtggtgta

SEQ ID NO: 119 - DNA, 43 bp, Artificial Sequence
  Description of Artificial Sequence Synthetic primer
atcaggcacaccggatttgcattaaagctgtcaagccgtgttc

SEQ ID NO: 120 - DNA, 42 bp, Artificial Sequence
  Description of Artificial Sequence Synthetic primer
caagcagaagacggcatacgaagaaaactccgcctttccagt

SEQ ID NO: 121 - DNA, 3203 bp, Artificial Sequence
  Description of Artificial Sequence Synthetic polynucleotide
aatgatacggcgaccaccgactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatcaagcatagcaggcacatcgcaatgtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagcctattgcaacagaggatgggccatggggcctgtgcaaggaagtattaccttcttataaatcaaactaaacatagctattctcatctgcattccaatgtgatgaaggccaaaaatggctgggtgtaggagcagtgtcctcacaataaagagaaggcataagcctatgcctagataaatcgcgatagagcgttcctccttgttatccgggtcataggaagctatgattcttcccagtaagagaggctgtactgctttggtgacttcctacaaaaggggaaaaacagagatgaggtttacacacggatccgaatcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggtcacacgatgcaggctgatagaatcttacctcttctagttggcatgctttgatgacgcttctgtatctatattcatcataggaaacaccaaagatgatattttctttaatggtgccaggcataatccaggaaaactgagaacagaatgaaattcttccactgtgcttaattttaccctctgaaggctccagttctcccattcacaatggctttccaggtcgagagcatactaaaagtgactctctaattttctatttttggtaataggacatctccaagtttgcagagaaagacaatatagttcttggagaaggtggaatcacactgagtggaggtcaacgagcaagaatttctttagcaaggtgaataactaattattggtctagcaagcatttgctgtagttaggttgtggagccaggagagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggtaatgtaccataatgctgcatctggacaggtctgtgcaaggagtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacaggtgagctgtctgcacgttcctacccacatctacactagatgaccaggaaatagagaggaaatgtaatttaatttccattttctttttagagcagtatacaaagatgctgatttgtatttattagactctccttttggatacctagatgttttaacagaaaaagaaatatttgaaaggtatgttctttgaataccttacttataatgctcatgtgcttagatcgactgggtgcgatacagacatacttaacggtacttatttttacatacctggatgaagtcaaatatggtaagaggcagaaggtcatccaaaattgctatatctttgttaccgtgccttcttcagctgatgaagaagatgacaaaaatcatttctattctcatttggaaccagcgcagtgttgacaggtacaagaaccagttggcagtatgtaaattcagagctttgtggaacagagtttcaaagtaaggctgccgtccgaaggcacgaagtgtccatagtccttttaagcttgtaacaagatgagtgaaaattggactcctgcctgtgaaatatttccatagaaaacattgcaaataacataaacacaaaatgtaatttcataatatcttgtacctgatcaggcgaaacgagttatgcgatggagagcataccagcagtgactacatggaacacataccttcgatatattactgtccacaagagcttaatttttgtgctaatttggtgcttagtaatttttctggcagatctttcagtcgcatggagcagtacctatttgctttacagcactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttctatcaggcacaccggatttgcattaaagctgtcaagccgtgttctagataaaataagtattggacaacttgttagtctcctttccaacaacctgaacaaatttgatgaagtatgtacctattgatttaatcttttaggcactattgttataaattatacaactggaaaggcggagttttcttcgtatgccgtcttctgcttg

SEQ ID NO: 122 - DNA, 43 bp, Artificial Sequence; identical to SEQ ID NO: 58
  Description of Artificial Sequence Synthetic primer

SEQ ID NO: 123 - DNA, 42 bp, Artificial Sequence; identical to SEQ ID NO: 70
  Description of Artificial Sequence Synthetic primer

SEQ ID NO: 124 - DNA, 49 bp, Artificial Sequence; identical to SEQ ID NO: 71
  Description of Artificial Sequence Synthetic primer

SEQ ID NO: 125 - DNA, 45 bp, Artificial Sequence; identical to SEQ ID NO: 72
  Description of Artificial Sequence Synthetic primer

SEQ ID NO: 126 - DNA, 38 bp, Artificial Sequence; identical to SEQ ID NO: 62
  Description of Artificial Sequence Synthetic primer

SEQ ID NO: 127 - DNA, 43 bp, Artificial Sequence; identical to SEQ ID NO: 74
  Description of Artificial Sequence Synthetic primer

SEQ ID NO: 128 - DNA, 45 bp, Artificial Sequence; identical to SEQ ID NO: 75
  Description of Artificial Sequence Synthetic primer

SEQ ID NO: 129 - DNA, 45 bp, Artificial Sequence; identical to SEQ ID NO: 87
  Description of Artificial Sequence Synthetic primer

SEQ ID NO: 130 - DNA, 47 bp, Artificial Sequence; identical to SEQ ID NO: 88
  Description of Artificial Sequence Synthetic primer

SEQ ID NO: 131 - DNA, 42 bp, Artificial Sequence; identical to SEQ ID NO: 89
  Description of Artificial Sequence Synthetic primer

SEQ ID NO: 132 - DNA, 52 bp, Artificial Sequence; identical to SEQ ID NO: 90
  Description of Artificial Sequence Synthetic primer

SEQ ID NO: 133 - DNA, 42 bp, Artificial Sequence; identical to SEQ ID NO: 104
  Description of Artificial Sequence Synthetic primer

SEQ ID NO: 134 - DNA, 46 bp, Artificial Sequence
  Description of Artificial Sequence Synthetic primer
cgttaggttgtggagccaggaacttcctttattttccttacagggt

SEQ ID NO: 135 - DNA, 43 bp, Artificial Sequence
  Description of Artificial Sequence Synthetic primer
caagcagaagacggcatacgactgctggtctgcctactagtga

SEQ ID NO: 136 - DNA, 1979 bp, Artificial Sequence
  Description of Artificial Sequence Synthetic polynucleotide
aatgatacggcgaccaccgactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatcaagcatagcaggcacatcgcaatgtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagcctattgcaacagaggatgggccatggggcctgtgcaaggaagtattaccttcttataaatcaaactaaacatagctattctcatctgcattccaatgtgatgaaggccaaaaatggctgggtgtaggagcagtgtcctcacaataaagagaaggcataagcctatgcctagataaatcgcgatagagcgttcctccttgttatccgggtcataggaagctatgattcttcccagtaagagaggctgtactgctttggtgacttcctacaaaaggggaaaaacagagatgaggtttacacacggatccgaatcttttgcagagaatgggatagagagctggcttcaaagaaaaatcctaaactcattaatgcccttcggcgatgttttttctggagatttatgttctatggaatctttttatatttaggggtaaggatctcatttgtacattcattatgtatcacataactatattcatttttgtgattatgaaaagactacgaaatctggtgaataggtcacacgatgcaggctgatagaatcttacctcttctagttggcatgctttgatgacgcttctgtatctatattcatcataggaaacaccaaagatgatattttctttaatggtgccaggcataatccaggaaaactgagaacagaatgaaattcttccactgtgcttaattttaccctctgaaggctccagttctcccattcacaatggctttccaggtcgagagcatactaaaagtgactctctaattttctatttttggtaataggacatctccaagtttgcagagaaagacaatatagttcttggagaaggtggaatcacactgagtggaggtcaacgagcaagaatttctttagcaaggtgaataactaattattggtctagcaagcatttgctgcgttaggttgtggagccaggaacttcctttattttccttacagggtttcagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttaaaaagttgaaaggttaatgtaaaacaatcaatattaaagaattttgatgccaaaactattagataaaaggttaatctacatccctactagaattctcatacttaactggttggttatgtggaagaaacatactttcacaataaagagctttaggatatgatgccattttatatcactagtaggcagaccagcagtcgtatgccgtcttctgcttg

SEQ ID NO: 137 - DNA, 41 bp, Artificial Sequence
  Description of Artificial Sequence Synthetic primer
gaggtttacacacggatccgatctggatcccagaaggtgag

SEQ ID NO: 138 - DNA, 42 bp, Artificial Sequence
  Description of Artificial Sequence Synthetic primer
tctatcagcctgcatcgtgtgcacagcaaagcagaaactcac

SEQ ID NO: 139 - DNA, 10 bp, Artificial Sequence
  Description of Artificial Sequence Synthetic primer tag
aggactgggg

SEQ ID NO: 140 - DNA, 57 bp, Artificial Sequence
  Description of Artificial Sequence Synthetic oligonucleotide
gtccttagtaccagattctgagcagggagaggcgacaagcatagcaggcacatcgca

SEQ ID NO: 141 - DNA, 61 bp, Artificial Sequence
  Description of Artificial Sequence Synthetic oligonucleotide
tttccaccttctgtgtattttgctgtgagatctttgacatgcgatgtgcctgctatgcttg

SEQ ID NO: 142 - DNA, 58 bp, Artificial Sequence
  Description of Artificial Sequence Synthetic oligonucleotide
gtccttagtaccagattctgagcagggagaggcgacaagcatagcaggcacatcgcaa

SEQ ID NO: 143 - DNA, 62 bp, Artificial Sequence
  Description of Artificial Sequence Synthetic oligonucleotide
tttccaccttctgtgtattttgctgtgagatctttgacatgcgatgtgcctgctatgcttga

SEQ ID NO: 144 - DNA, 59 bp, Artificial Sequence
  Description of Artificial Sequence Synthetic oligonucleotide
gtccttagtaccagattctgagcagggagaggcgatcaagcatagcaggcacatcgcaa

SEQ ID NO: 145 - DNA, 63 bp, Artificial Sequence
  Description of Artificial Sequence Synthetic oligonucleotide
tttccaccttctgtgtattttgctgtgagatctttgacattgcgatgtgcctgctatgcttga

SEQ ID NO: 146 - DNA, 89 bp, Artificial Sequence
  Description of Artificial Sequence Synthetic oligonucleotide
catactaaaagtgactctctaattttctatttaggacaagagatatccaattggagagtggatagataactaattattggtctagcaag

SEQ ID NO: 147 - DNA, 89 bp, Homo sapiens
catactaaaagtgactctctaattttctatttaggacaagagaaagacaattggagagtggatagataactaattattggtctagcaag

SEQ ID NO: 148 - DNA, 94 bp, Artificial Sequence
  Description of Artificial Sequence Synthetic oligonucleotide
tttaacttcctttattttccttacagggtttgagacaatttatggtttgtggtttacaaatgttataaagttgaaaggttaatgtaaaacaatc

SEQ ID NO: 149 - DNA, 94 bp, Homo sapiens
tttaacttcctttattttccttacagggtttcagacaatttatggtttgtggaaaacaaatgttaaaaagttgaaaggttaatgtaaaacaatc

SEQ ID NO: 150 - DNA, 94 bp, Homo sapiens
tttaacttcctttattttccttacagggttttagacaatttatggtttgtggaaaacaaatgttagaaagttgaaaggttaatgtaaaacaatc

SEQ ID NO: 151 - DNA, 136 bp, Artificial Sequence
  Description of Artificial Sequence Synthetic polynucleotide
gtgtggtctcccataccctctcagcgtacccttgtccccaggaagcatacgtgatggctggtgtgggctccccatatgtctcccgccttctgggcatctgcctgacatccacggtgcagctggtgacacagcttat